(19) United States
(12) Patent Application Publication
(10) Pub. No.: US 2003/0032189 A1
(43) Pub. Date: Feb. 13, 2003

(54) Method and apparatus for calculating dew point, method and apparatus for compensating for dew point, MOS gas sensor system, and fuel cell system

(76) Inventors: Greg A. Lloyd, Spokane, WA (US); Howard T. Voorheis, Everett, WA (US); William A. Fuglevand, Spokane, WA (US)

Correspondence Address: WELLS ST. JOHN ROBERTS GREGORY & MATKIN P.S., 601 W. FIRST AVENUE, SUITE 1300, SPOKANE, WA, 99201-3828, US

(21) Appl. No.: 09/987,540

(22) Filed: Nov. 15, 2001

Related U.S. Application Data
(63) Continuation-in-part of application No. 09/854,059, filed on May 11, 2001.

(51) Int. Cl. G01N 33/18

(52) U.S. Cl. 436/39; 429/22; 436/151; 422/88

(57) ABSTRACT

A method for calculating dew point comprises providing a signal representative of relative humidity of the ambient; calculating a temperature signal representative of a predetermined dew point; providing a signal representative of actual temperature of the ambient; determining the difference between the signal representative of actual temperature and the calculated temperature signal to provide a differential temperature; using the relative humidity signal, calculating the rate at which dew point changes as a function of temperature; and calculating dew point by multiplying the differential temperature by the calculated rate at which dew point changes as a function of temperature. An apparatus for calculating dew point includes digital or analog circuitry for performing similar calculations.

### RELATED PATENT DATA

This application is a continuation-in-part of U.S. patent application Ser. No. 09/854,059, which was filed on May 11, 2001 and which is incorporated by reference herein.

### TECHNICAL FIELD

The present invention relates to gas sensors and more specifically to compensating gas sensors for the effects of temperature and humidity. The invention also relates to metal oxide semiconductor gas sensors and fuel cell systems including gas sensors.

### BACKGROUND OF THE INVENTION

For safety purposes, gas-sensing instruments are used in many industrial applications such as in fuel cell systems whose feedstocks are flammable gases. It is well known that many gas sensors—metal oxide semiconductor (MOS) based sensors in particular—suffer from environmental dependencies. That is, ambient temperature and relative humidity substantially affect their sensitivity. For example, one commercially available MOS sensor model is the Figaro TGS821 hydrogen sensor. Due to the combination of this sensor's environmental dependencies and the environmental uncertainties to which it will be exposed to in certain fuel cell applications, a sensor reporting a reading of 526 PPM of hydrogen might actually be exposed to a true concentration ranging between 182 and 1627 PPM. In certain fuel cell applications, the lower reading would be regarded as being well below alarm-level whereas the higher reading would be regarded as being well above. This 8.9:1 range of uncertainty is the source of much frustration with uncompensated MOS gas sensors.

Accordingly, many designers of gas sensing instruments elect to compensate for MOS gas sensors' environmental dependencies. The conventional wisdom is that this requires a microprocessor, firmware (software), and lookup charts. However, dependence upon firmware being perpetually executed without error in a microprocessor-based circuit greatly complicates efforts to design a highly reliable, gas-sensing instrument. Furthermore, the conventional method produces compensation factors that are inexact approximations of the required values.

Attention is directed toward the following U.S. patents, which are incorporated herein by reference: U.S. Pat. Nos. 5,716,506 to Maclay et al.; 4,313,338 to Abe et al.; 4,801,211 to Yagietal.; 6,126,311 to Schuh; and 5,969,231 to Qu et al.

U.S. Pat. No. 5,716,506 to Maclay et al. discloses (see Col. 1) a gas sensor that compensates for relative humidity and temperature of the air in the detection of a predetermined gas in a microfabricated electrochemical sensor.

U.S. Pat. No. 4,313,338 to Abe et al. relates to a gas sensing device comprising a resistive film formed of ultra fine particles of a metal oxide (Col. 4, lines 10-15). The gas sensing device includes (Col. 7, line 43-Col 8, line 65) a temperature sensing element for maintaining the temperature of the gas sensitive element constant. U.S. Pat. No. 4,313,338 also discloses obviating the problem of water vapor obstructing the successful measurement of the concentration of gas by using a single gas sensing element to sense both the concentration of water vapor and the concentration of isobutane gas (see Col. 8, line 47-Col. 9, line 11). The gas sensing element is heated up to 300° C. during the measurement of the concentration of the isobutane gas and is cooled down to the room temperature of 25° C. during the measurement of relative humidity.

U.S. Pat. No. 4,801,211 to Yagi et al. discloses (see Abstract) a humidity sensor that, when temperature corrected, indicates a dew point at a fixed temperature. By adjusting this fixed temperature dew point output according to a sensed temperature, the dew point can be detected. FIG. 2 shows all analog circuitry. The sensor is made of metal oxide ceramic material (see Col. 4, lines 44-46).

U.S. Pat. No. 6,126,311 to Schuh discloses (see FIG. 4) a sensor that outputs dew point, ambient temperature, and relative humidity. This patent discloses (see Col. 1, lines 14-20) that the relative humidity and dew point of a gaseous sample are closely related by well known algorithms for converting dew point and ambient temperature to relative humidity or converting relative humidity and ambient temperature to dew point. This patent also indicates (see Col. 2, lines 19-23) that a group of prior art sensors measure the relative humidity of an ambient environment as opposed to dew point, and that relative humidity and dew point are easily converted from one to the other with a measurement of the ambient air temperature.

U.S. Pat. No. 5,969,231 to Qu et al. discloses a sensor for monitoring the concentration of moisture and gaseous substances in the air. Semiconductive metal oxides are used (see Col. 1).

Notwithstanding the prior art teachings noted above, none of these references singularly or in any permissible combination teach a simple approach for compensating gas sensor measurements for both humidity and temperature at the same time. It would be advantageous therefore, to be able to perform such compensation utilizing analog circuitry, which would be highly reliable.

Dew point is determined, in the prior art, using expensive chilled-mirror equipment. It would be advantageous to be able to determine dew point in a less expensive manner.

As noted above, gas sensors are used, in various industrial applications, such as in the fabrication of fuel cells. For example, gas sensors configured to sense hydrogen can be employed to detect hydrogen fuel leaks or hydrogen fuel flow in the fuel cells. In this regard, attention is directed to commonly assigned U.S. patent application Ser. No. 09/322,666 filed May 28, 1999, listing as inventors Fuglevand et al., and which is incorporated by reference herein. This application discloses the particulars of how gas sensors can be employed in one form of a fuel cell system.

### BRIEF DESCRIPTION OF THE DRAWINGS

Preferred embodiments of the invention are described below with reference to the following accompanying drawings.

FIG. 1 is a perspective, side elevation view of an ion exchange membrane fuel cell module which is utilized with a fuel cell power system embodying the present invention.

FIG. 2 is a perspective, exploded, side elevation view of an ion exchange membrane fuel cell module as seen in FIG. 1.

FIG. 3 is a perspective, partial, exploded, side elevation view of an ion exchange membrane fuel cell module as seen in FIG. 1.

FIG. 4 is a fragmentary, perspective, greatly enlarged, exploded view of a membrane electrode diffusion assembly employed with the ion exchange membrane fuel cell module as seen in FIG. 1.

FIG. 5 is a fragmentary, side elevational view of a fuel distribution assembly utilized with the ion exchange membrane fuel cell module as seen in FIG. 1.

FIG. 6 is a second, fragmentary, side elevational view of the fuel distribution assembly taken from a position opposite to that seen in FIG. 5.

FIG. 7 is a second, perspective, partial, exploded view of a portion of the ion exchange membrane fuel cell module as seen in FIG. 1.

FIG. 8 is a perspective view of an ion exchange membrane fuel cell subrack and associated fuel gas supply.

FIG. 9 is a fragmentary, transverse, vertical sectional view taken from a position along line 9-9 of FIG. 8.

FIG. 10 is a fragmentary, schematic representation of an ion exchange membrane fuel cell module, and associated power system.

FIG. 11 is a graph illustrating the average ratiometeric response of a typical MOS gas sensor to the concentration of a gas used as a fuel in the fuel cell system as illustrated.

FIG. 12 is a graph illustrating the effects of temperature and humidity on the same MOS gas sensor.

FIG. 13 is a graph illustrating dew points plotted against Rs/Ro values for the same MOS sensor.

FIG. 14 is a graph illustrating environmental Rs/Ro relationships using dew points for the same MOS gas sensor.

FIG. 15 is a graph illustrating the relationship of compensation factors vs. water content for the same MOS gas sensor.

FIG. 16 is a block diagram illustrating the circuitry utilized to transform a dew point signal into a linear or semi-linear compensation factor and multiply that same compensation factor by the output of the MOS gas sensor.

FIG. 17 is a graph illustrating the relationship of temperature and relative humidity to dew point.

FIG. 18 is a block diagram of circuitry used to determine dew point from relative humidity and temperature as well as circuitry to transform the dew point signal into a linear or semi-linear compensation factor for the MOS gas sensor.

FIG. 19 is a graph illustrating percentage error in one formula for determining dew point from temperature and humidity.

FIG. 20 is a flowchart illustrating logic used by digital circuitry or by a programmed general purpose computer or a processor for determining dew point from relative humidity and temperature.

FIG. 21 is a map showing how FIGS. 21A and 21B are to be assembled. When assembled, FIGS. 21A and 21B show a block diagram of circuitry for providing a linear output signal from a hydrogen sensor.

FIG. 22 shows a block diagram of circuitry for providing the correction factor K of FIG. 16.

FIGS. 23A and 23B when assembled provide a circuit schematic of circuitry that could be used to define an lo supply, hydrogen sensor, and buffer of FIGS. 21A-B.

FIGS. 24A and 24B when assembled provide a circuit schematic of circuitry that could be used to define a current supply, logarithmic amplifier, temperature compensating amp, and inverting amp of FIGS. 21A-B.

FIGS. 25A, 25B, and 25C when assembled provide a circuit schematic of circuitry that could be used to define a summing amplifier, divider, and inverting amplifiers shown in FIGS. 21A-B.

FIG. 26 is a circuit schematic of circuitry for generating a correction factor CF, and circuitry that can be used to define summing amplifiers of FIGS. 21A-B.

FIGS. 27A, 27B, and 27C when assembled provide a circuit schematic of circuitry that could be used to define a current supply, an anti-log amplifier, a gain amplifier, and a summing amplifier of FIGS. 21A-B.

FIGS. 28A and 28B when assembled provide a circuit schematic of circuitry for generating a voltage VH, circuitry defining a function b(T), and circuitry defining a summing amplifier.

FIGS. 29A-F when assembled provide a circuit schematic of circuitry defining a current source, a logarithmic amplifier, a multiplier, and circuitry defining the function a(T).

FIGS. 30A-B when assembled provide a circuit schematic of circuitry for generating a signal representative of the temperature of the plenum.

FIG. 31 is a circuit schematic of circuitry for providing a signal representative of temperature of the humidity sensor

FIGS. 32A-B when assembled provide a circuit schematic of a current source, a logarithmic amplifier, a temperature compensating amplifier, and an inverting amplifier.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

The present invention provides a method of calculating dew point, comprising providing a signal representative of relative humidity of the ambient; calculating a temperature signal representative of a predetermined dew point; providing a signal representative of actual temperature of the ambient; determining the difference between the signal representative of actual temperature and the calculated temperature signal to provide a differential temperature; using the relative humidity signal, calculating the rate at which dew point changes as a function of temperature; and calculating dew point by multiplying the differential temperature by the calculated rate at which dew point changes as a function of temperature.

Another aspect of the present invention provides a method of calculating and compensating for dew point, comprising providing a signal representative of relative humidity of the ambient; calculating a temperature signal representative of a predetermined dew point; providing a signal representative of actual temperature of the ambient and determining the difference between the signal representative of actual temperature and the calculated temperature signal to provide a differential temperature; using the relative humidity signal, calculating the rate at which dew point changes as a function of temperature; calculating dew point by multiplying the differential temperature by the calculated rate at which dew point changes as a function of temperature; providing a signal indicative of a gas concentration of a target gas in an ambient; and modifying the signal indicative of the gas concentration using the calculated dew point to simultaneously compensate for the effects of both temperature and relative humidity on a MOS gas sensor.

Another aspect of the present invention relates to a system for calculating dew point, comprising a humidity sensor which, in operation, provides a signal representative of relative humidity of the ambient; circuitry configured to calculate a temperature signal representative of a predetermined dew point; a temperature sensor which, in operation, provides a signal representative of temperature of the ambient; circuitry which, in operation, determines the difference between the calculated temperature signal and the signal representative of the temperature of the ambient to provide a differential temperature; circuitry which, in operation, using the relative humidity signal, calculates the rate at which dew point changes as a function of temperature; and circuitry which, in operation, calculates dew point by multiplying the differential temperature by the calculated rate at which dew point changes as a function of temperature.

Another aspect of the present invention relates to a MOS gas sensor system comprising a MOS gas sensor which, in operation, provides a signal representative of the concentration of a target gas in an ambient; a humidity sensor which, in operation, provides a signal representative of relative humidity of the ambient; circuitry configured to calculate a temperature signal representative of a predetermined dew point; a temperature sensor which, in operation, provides a signal representative of temperature of the ambient; circuitry which, in operation, determines the difference between the calculated temperature signal and the signal representative of the temperature of the ambient to provide a differential temperature; circuitry which, in operation and using the relative humidity signal, calculates the rate at which dew point changes as a function of temperature; dew point calculation circuitry which, in operation, calculates dew point by multiplying the differential temperature by the calculated rate at which dew point changes as a function of temperature; and compensation circuitry coupled to the dew point calculation circuitry and configured to modify the signal from the MOS gas sensor, using the calculated dew point, to simultaneously compensate for the effects of both temperature and relative humidity on the MOS gas sensor.

Another aspect of the invention relates to a fuel cell system comprising a housing having a fuel gas inlet and an exhaust outlet; at least one ion exchange fuel cell membrane located within the housing; and a MOS gas sensor system including a MOS gas sensor which, in operation, provides a signal representative of the concentration of a target gas in an ambient; a humidity sensor which, in operation, provides a signal representative of relative humidity of the ambient; circuitry configured to calculate a temperature signal representative of a predetermined dew point; a temperature sensor which, in operation, provides a signal representative of temperature of the ambient; circuitry which, in operation, determines the difference between the calculated temperature signal and the signal representative of the temperature of the ambient to provide a differential temperature; circuitry which, in operation, using the relative humidity signal, calculates the rate at which dew point changes as a function of temperature; dew point calculation circuitry which, in operation, calculates dew point by multiplying the differential temperature by the calculated rate at which dew point changes as a function of temperature; and compensation circuitry coupled to the dew point calculation circuitry and configured to modify the signal from the MOS gas sensor, using the calculated dew point, to simultaneously compensate for the effects of both temperature and relative humidity on the MOS gas sensor.

Another aspect of the invention relates to analog circuitry for calculating dew point, comprising a humidity sensor configured to provide a signal representative of relative humidity of the ambient; a temperature sensor configured to provide a signal representative of temperature of the ambient; and analog circuitry, coupled to the humidity sensor and the temperature sensor, configured to provide a signal representative of dew point using analog circuitry modeled on a least squares fit to a formula that relates temperature and relative humidity to dew point.

Another aspect of the invention relates to a method of calculating dew point, comprising providing a signal representative of relative humidity of the ambient; providing a signal representative of temperature of the ambient; and calculating the rate at which dew point changes as a function of temperature and humidity using analog circuitry modeled to implement an equation of the form K(T,H)=(a₁T+a₀)log₁₀[(b₁T+b₀)+H]+(c₁T+c₀) where K(T,H) is a scaled dew point temperature, where T is ambient temperature, where a₀, a₁, b₀, b₁, c₀, and c₁ are constants, and where H is relative humidity.

Another aspect of the invention relates to a method of calculating dew point, comprising sensing the relative humidity of the ambient; sensing the temperature of the ambient; and determining the rate at which dew point changes as a function of temperature and humidity using a transfer function of the form Y=MX+B which can be plotted as a line segment on a graph having x and y axes, wherein b is a dynamic constant, is a first-order function of temperature that determines the y-axis origin of the line segment mx, and represents a reference point on a dew point scale; wherein x is a multi-variable representation of x-axis displacement from the origin and is a first-order log (base 10) function of both temperature and humidity; wherein m is a dynamic constant that determines the slope of the line segment and is a first-order function of temperature; and wherein y is the difference relative to b and represents dew point temperature.

Another aspect of the invention relates to a method of compensating a MOS gas sensor for environmental dependencies, where said MOS gas sensor has a transfer function that is substantially linear when viewed on a log₁₀−log₁₀ plot of a signal representative of the sensor's output versus gas concentration, the transfer function having aslope representing raw gain and having a raw offset, the method comprising taking the raw offset and gain of the sensor's transfer function, and generating a first signal by normalizing the raw offset and gain to fit a predetermined transfer function at a predetermined dew point, and wherein the sensor's first signal's gain is scaled on a log₁₀ basis; taking a second signal, which is a function of the dew point of the gas which the MOS gas sensor is configured to sense, and where the influence of said second signal on the first signal is null at a predetermined dew point; and summing the first and second signals so as to offset the first signal upwards or downwards as viewed on a log₁₀−log₁₀ plot.

Another aspect of the invention relates to a method of calculating dew point (t′) given inputs of relative humidity (H) and temperature (T), using a transfer function t′=MX+B; wherein B is a function of temperature; M is a function of temperature; and X is a log base 10 function of relative humidity and temperature.

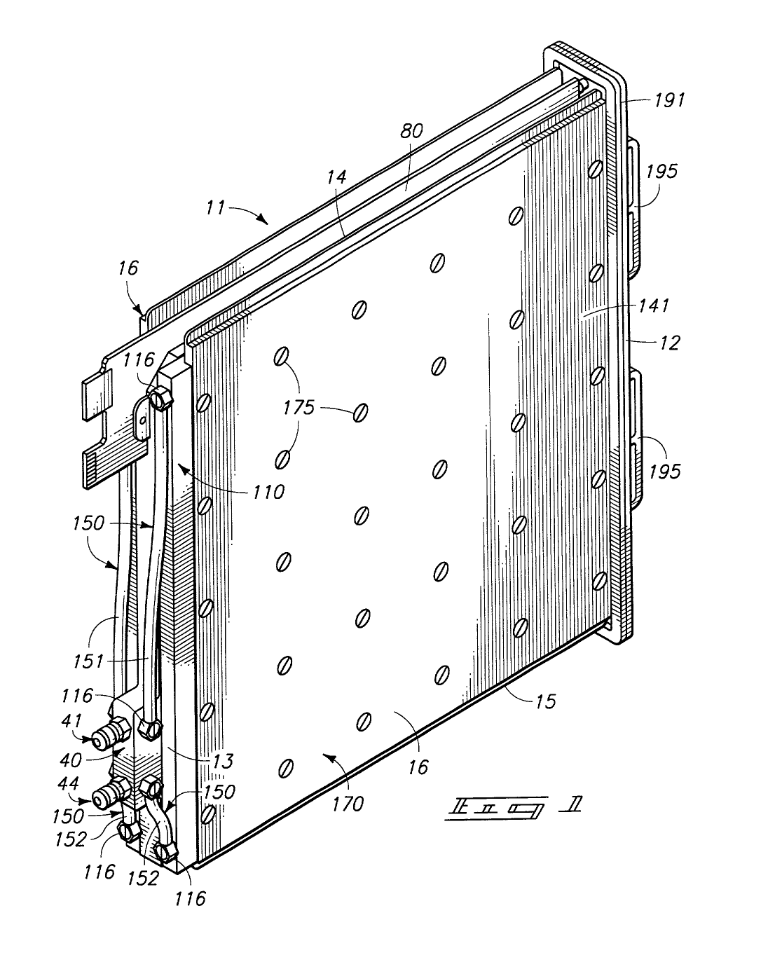

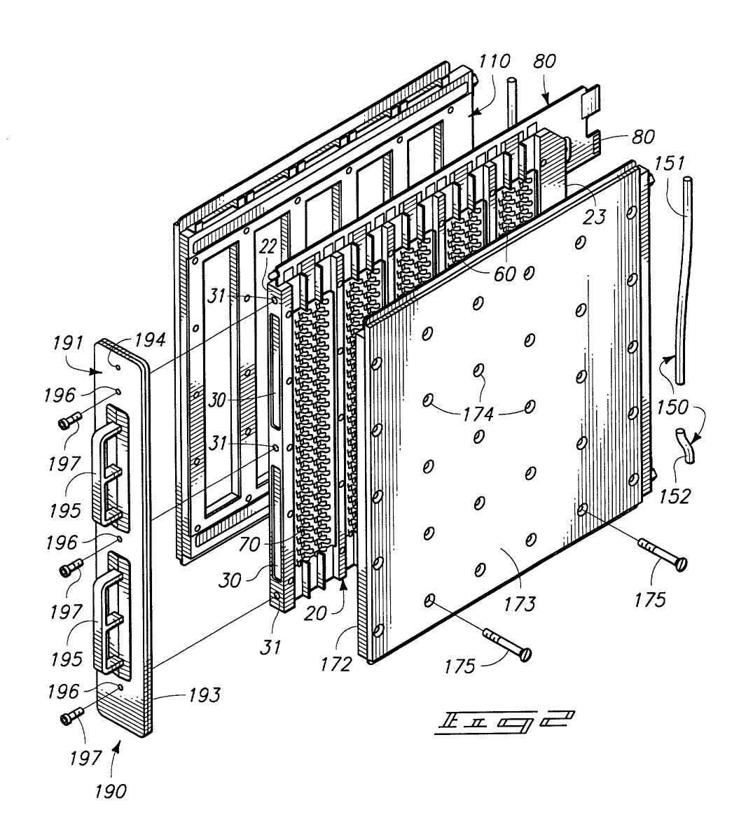

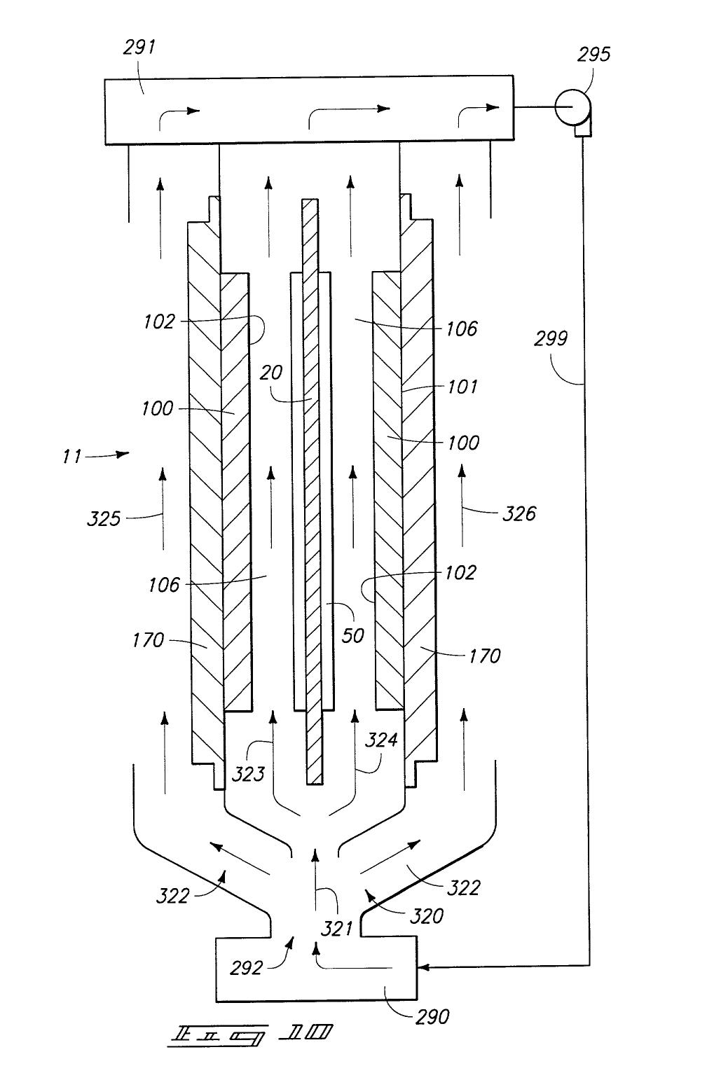

As best seen in FIG. 8, an ion exchange membrane fuel cell power system 10 is made up of a plurality of fuel cell modules 11, one of which is shown in FIG. 1. The ion exchange membrane fuel cell power system 10 may include a plurality of subsystems or subracks 210. As illustrated each subsystem or subrack 210 includes a plurality of hand manipulable modules 11 (FIG. 1) which respectively have a forward edge 12, an opposite, rearward edge 13, top and bottom surfaces or edges 14 and 15, and opposite sidewalls generally indicated by the numeral 16. Each facet of the module 11 will be discussed in greater detail hereinafter. Yet further those should recognize that the present invention could be employed with conventional stack-like technology wherein the individual subsystem comprises fuel cell stacks arranged in a manner which is consistent with the further teachings of this application.

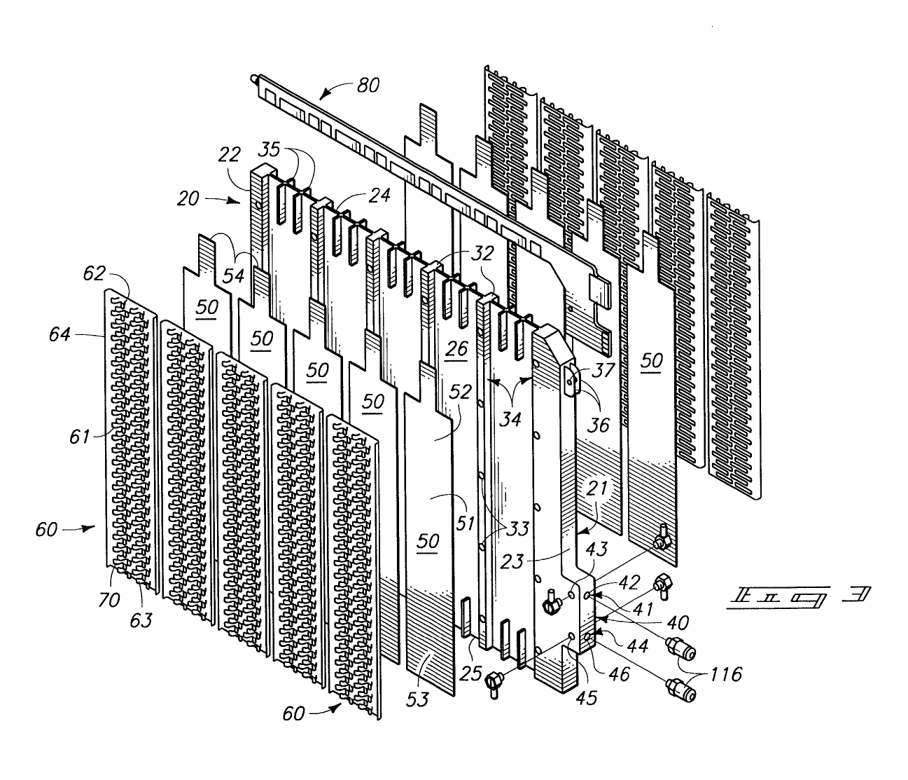

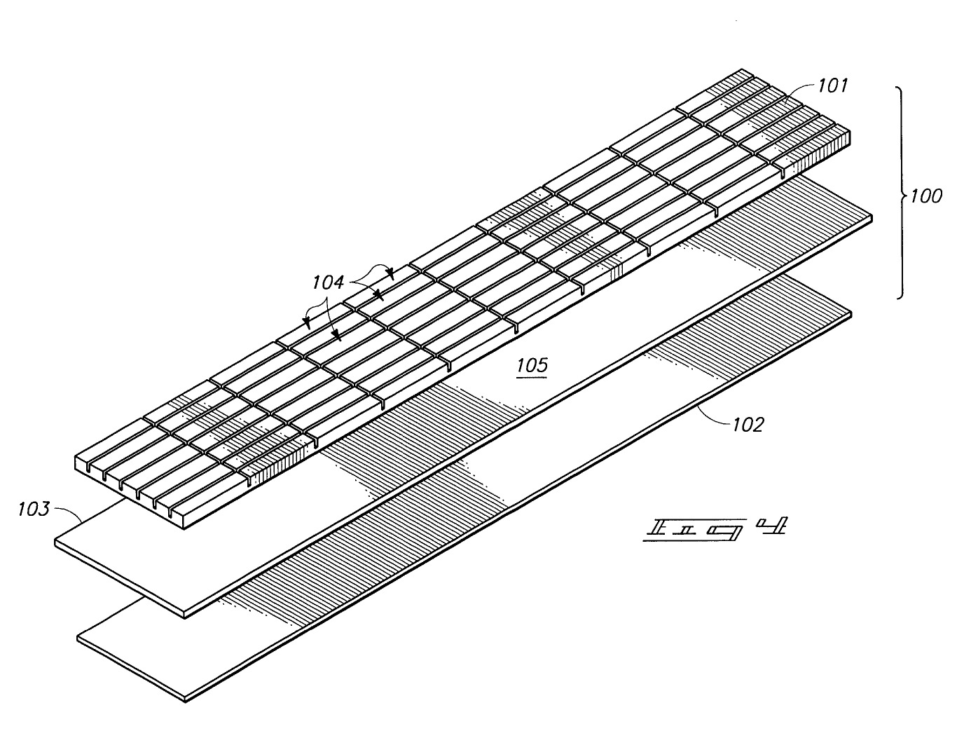

As best seen in FIGS. 2 and 3, the fuel cell module 11 includes a nonconductive, dielectric support member generally indicated by the numeral 20. The support member can be fashioned out of various synthetic polymeric substrates. The support member has (see FIG. 3) a main body 21, which is defined by a forward peripheral edge 22; a rearward peripheral edge 23; a top peripheral edge 24; an opposite, bottom peripheral edge 25; and opposite sidewalls generally indicated by the numeral 26.

As best seen in FIG. 2, a pair of recessed channels 30 are formed in the forward peripheral edge 22. Further, a plurality of fastener receiving passageways or apertures 31 are also formed in the forward peripheral edge 22. Yet further, and as seen in FIG. 3, a plurality of spaced ribs 32 are borne by, or made integral with the respective sidewalls 26 and are disposed in spaced relation, one to the other. Fastener passageways or apertures 33 are formed through each of the ribs. Further, cavities 34 are defined between the respective ribs 32 on each sidewall. The cavities 34 formed on each of the sidewalls are disposed in substantially opposed relation one to the other. This is seen in FIG. 3.

Further, as best seen in FIG. 3, orientation members 35 are disposed between each of the ribs 32 and define a space therebetween. A pair of mounting tabs 36 are provided in spaced relationship, one to the other, on the rearward peripheral edge 23 of the main body 21. A pair of substantially coaxially aligned apertures 37 are individually formed in each of the mounting tabs 36 and are operable to receive a fastener therethrough.

A fuel coupling 40 is made integral with or forms a portion of the rearward peripheral edge 23 of the support member 20. The fuel coupling 40 includes a fuel delivery passageway 41 which is substantially T shaped and which is defined by an intake end 42 and a pair of exhaust ends labeled 43. Additionally, the fuel coupling also includes an exhaust passageway 44 which is also substantially T shaped and which is defined by a pair of intake ends 45, and an exhaust end 46. The operation of the fuel coupling 40 will be discussed in greater detail hereinafter.

As best seen in FIGS. 2 and 3, individual conductor plates generally designated by the numeral 50 are matingly received within the individual cavities 34 which are defined by the support member 20. The conductor plates which are fabricated from an electrically conductive substrate, have a substantially planar main body 51, which has a first end 52, and an opposite, second end 53. The main body 51 further has a conductive tab 54 which extends outwardly relative to the first end 52, and which is oriented between the individual orientation members 35. The conductive tab extends substantially normally outwardly relative to the top peripheral edge 24 of the support member 20. As will be recognized, the main body 51 matingly rests between the individual ribs 32 which define, in part, the respective cavities 34.

As best seen in the exploded view of FIG. 3, a cathode current collector is generally designated by the numeral 60, and rests in ohmic electrical contact with the main body 51 of the individual conductor plates 50. The cathode current collector, which is fabricated from an electrically conductive substrate, has a main body 61 which has opposite first and second ends 62 and 63, respectively. The cathode current collector simultaneously performs the functions of current collection, force application and heat dissipation. Still further, the main body 61 of the current collector 60 is defined by a peripheral edge 64.

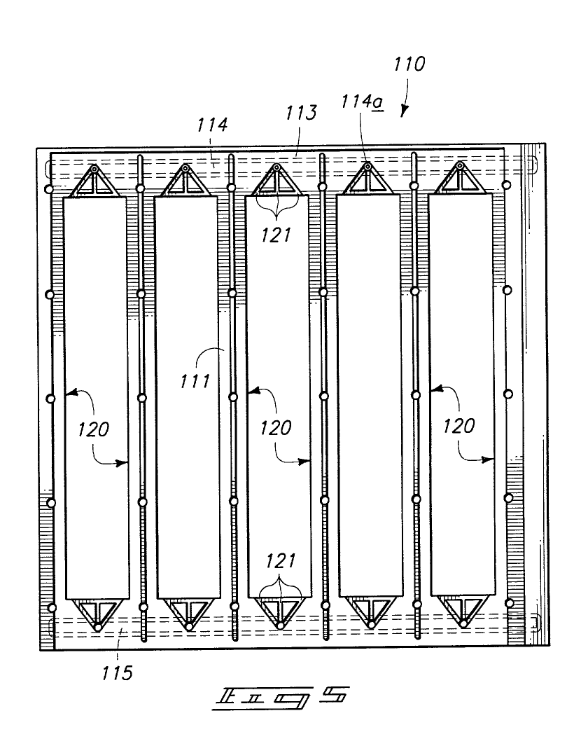

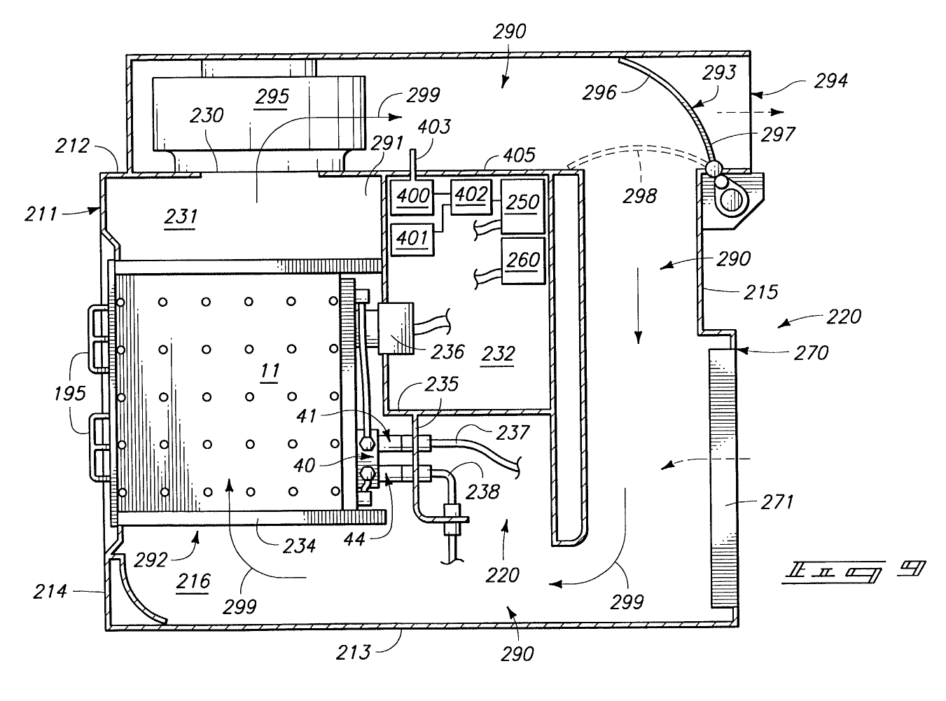

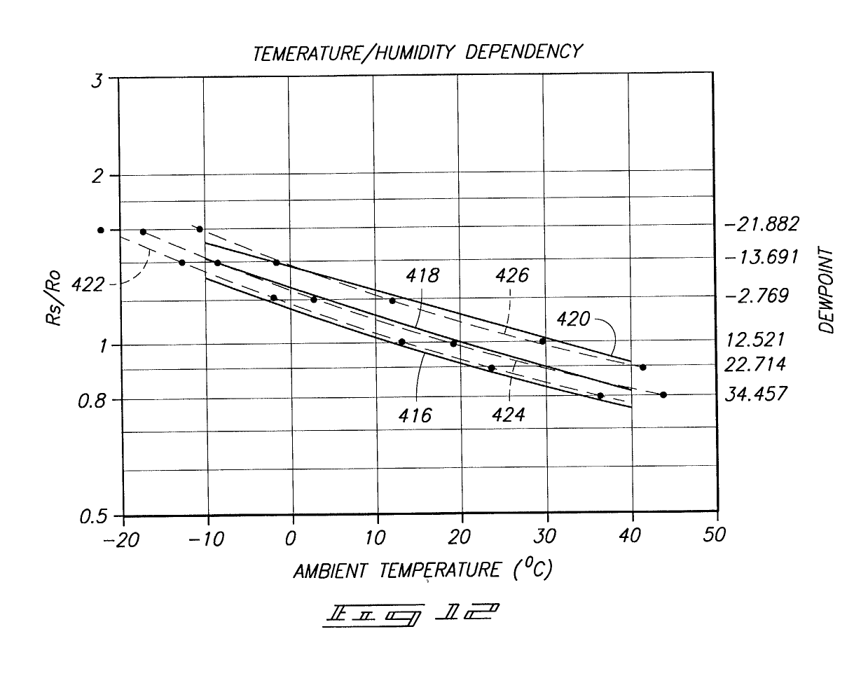

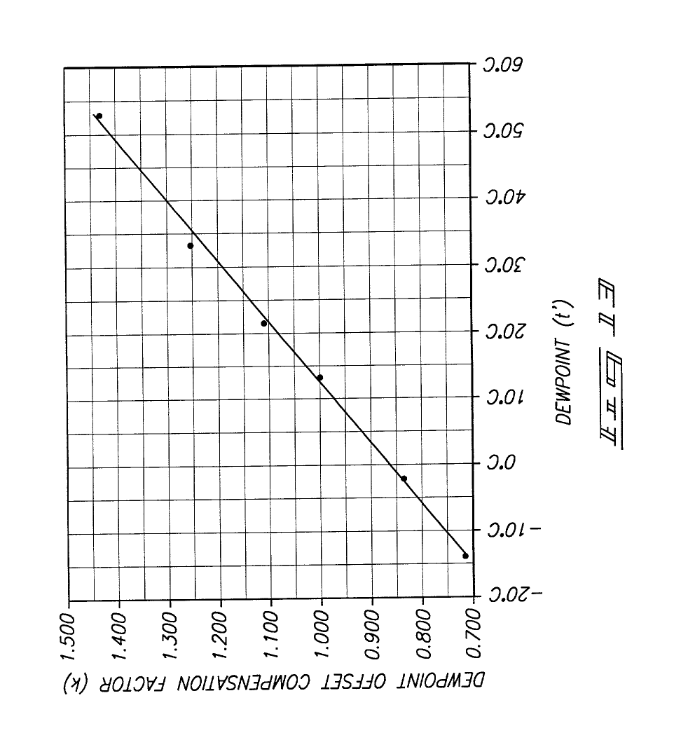

As best seen in the exploded view of FIGS. 4 and 7, the ion exchange membrane fuel cell module 11 includes a plurality of membrane electrode diffusion assemblies generally indicated by the numeral 100. Each of the membrane electrode diffusion assemblies have an anode side 101, and an opposite cathode side 102. Still further, each of the membrane electrode diffusion assemblies is defined by a peripheral edge 103, and further has formed in its anode side, a plurality of interlinking channels 104. The membrane electrode diffusion assembly 100, as noted above, is formed of a solid ion conducting membrane 105 which is sealably mounted or received in each of the respective cavities 34. In this arrangement, the cathode side 102 of each membrane electrode diffusion assembly 100 is held in spaced relation relative to the support member 20 by deformable electrically conductive members 70 (FIGS. 2 and 3) of the cathode current collector 60. This spacial arrangement, which is provided by the cathode current collector, facilitates, in part heat dissipation from the fuel cell module 11. As described, above, the membrane electrode diffusion assembly 100; associated cathode current collector 60; and support member 20 in combination, define a cathode air passageway 106 therebetween (FIG. 10). The construction of a suitable membrane electrode diffusion assembly was described in our earlier U.S. Pat. No. 6,030,718. This earlier patent is incorporated by reference herein, and further discussion regarding the construction of the membrane electrode diffusion assembly is not undertaken herein.

As will be appreciated, from a study of FIG. 10, the cathode air passageway 106 is defined or otherwise oriented on each side 26 of the support member 20. Therefore, the fuel cell module 11 has a bifurcated cathode air flow. As will be appreciated, while the earlier described membrane electrode diffusion assembly was directed to a proton exchange membrane, the fuel cell power system 10 is not limited solely to a type having proton exchange membranes, but also may utilize anion exchange membranes.

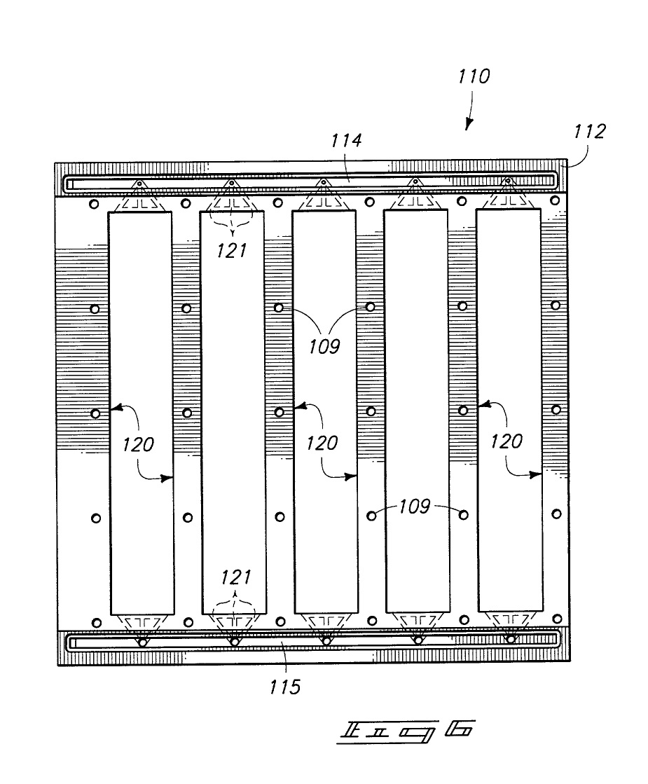

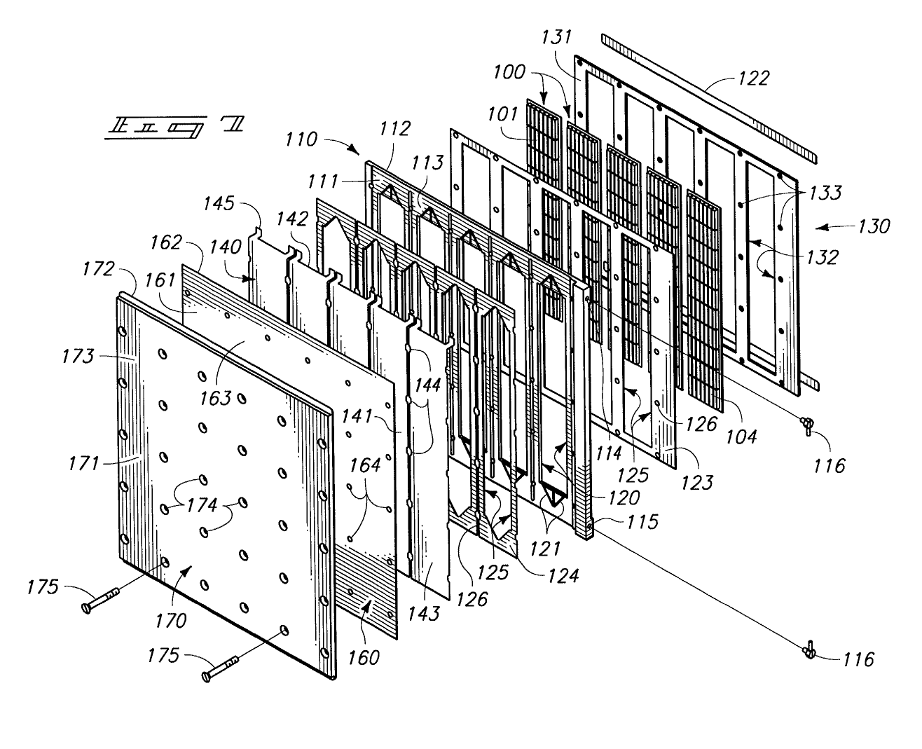

As best seen by reference to FIGS. 5, 6 and 7, a fuel distribution assembly, which is generally indicated by the numeral 110, is coupled in fluid flowing relation relative to the anode side 101 of each of the membrane electrode diffusion assemblies 100. Each fuel distribution assembly 110 is coupled with a source of a fuel 340 (FIG. 8) which may be substantially pure, or which is diluted to various degrees. Such may be achieved if the fuel cell power system was coupled with a fuel processor which would produce a stream of hydrogen from a source of hydrocarbon fuel such as gasoline, natural gas, propane, etc. If the fuel cell power system 10 was fabricated in the nature of a proton exchange membrane fuel cell, the dilute fuel supply would include hydrogen. The concentration of the hydrogen in the dilute fuel would normally be in a range of about 30% to about 80% by volume.

When supplied with this dilute fuel mixture (regardless of the type), the fuel cell modules 11 produce an average current density of at least about 350 mA per square centimeter of surface area of each anode side 101 at a nominal voltage of 0.5 volts. Further, the interlinking channels 104 formed in the surface of the anode side 101 facilitate the distribution of the dilute fuel substantially about the entire surface area of the anode side 101. In this arrangement, if contaminants are introduced by way of the dilute fuel mixture or other blockage occurs, the interlinking channels 104 provide a convenient passage by which the fuel may reach substantially the entire surface area of the anode side 101, even though some portions of the interlinking channels 104 may be blocked or otherwise substantially occluded. As noted above, the dilute fuel 340 may be supplied by a fuel processor 342 (FIG. 8) which receives a hydrocarbon based fuel, and then through a chemical reaction fractionates the hydrocarbon fuel source to liberate a dilute stream of hydrogen which is mixed with other substances. In the alternative, the fuel may be supplied by a pressurized container 341. These alternative arrangements are shown in FIG. 8.

As best seen by reference to the exploded view as shown in FIG. 7 and FIG. 1, the ion exchange membrane fuel cell module of the present invention includes a pair of the fuel distribution assemblies 110 which are individually mounted in fluid flowing relation relative to the anode side 101 of the respective membrane electrode diffusion assemblies 100.

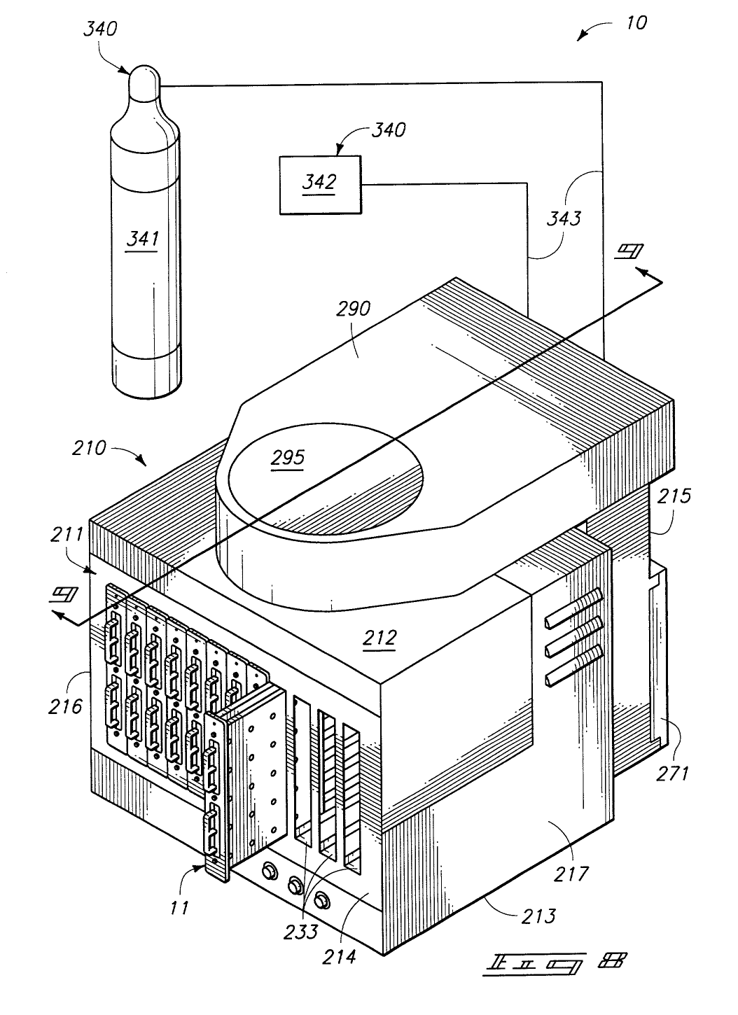

As best seen in FIGS. 5 and 6, each of the fuel distribution assemblies 110 include a main body 111 which has an inside facing surface 112, (FIG. 6) and an outside facing surface 113 (FIG. 5). The main body 111 further defines an intake plenum 114, and an exhaust plenum 115. Further, a fluid coupling 116 (FIG. 1) is mounted in fluid flowing relation relative to the individual intake and exhaust plenums 114 and 115 respectively. A reduced dimension orifice 114a (FIG. 5) is formed in the main body and communicates with the intake plenum 114. This reduced diameter orifice operates to create a pressure differential in the respective apertures or cavities 120 during certain operational conditions to facilitate the clearance of contaminants or other obstructions which may be blocking any of the channels 104 which are formed in the membrane electrode diffusion assembly 100. A plurality of cavities or apertures 120 are formed in the main body 111, and extend between the inside and outside facing surfaces 112 and 113 respectively. The cavities or apertures 120 are disposed in spaced relation, one to the other, and when assembled, the cavities 120 receive the individual membrane electrode diffusion assemblies 100. As best seen in FIG. 5, a plurality of channels or passageways 121 are formed in the main body 111, and couple the individual cavities 120 in fluid flowing relation with the respective intake and exhaust plenums 114 and 115. Additionally, a plurality of fastener apertures 109 are formed in the main body. As further seen in FIG. 7, a sealing member 122 lies in covering relation relative to the individual channels 121.

As best seen in FIG. 1, a plurality of conduits 150 couple in fluid flowing relation the fuel coupling 40 with the fuel distribution assembly 110. Two of the conduits designated as 151 allow a dilute fuel mixture to be delivered by way of the intake plenum 114 to the individual membrane electrode diffusion assemblies 100. Thereafter, any remaining fuel, and associated by-products of the chemical reaction are received back into the exhaust plenum 115 and then flow by way of conduits 152 to the fuel coupling 40 and then to the exhaust passageway 44.

First and second pressure sensitive adhesive seals 123 and 124 (FIG. 7), respectively are provided, and are disposed in juxtaposed relation relative to the opposite inside and outside facing surfaces 112 and 113 respectively. Each of the seals 123 and 124 have apertures 125 formed therein which are substantially coaxially oriented relative to the respective cavities 120. As will be recognized, the cavities 120 which are formed in the main body 111 of the fuel distribution assembly 110, matingly cooperate and are substantially coaxially aligned with the individual cavities 34 which are formed in the nonconductive support plate 20. As will be recognized and following the assembly of same, the respective membrane electrode diffusion assemblies 100 are individually received in mating relation in each of the cavities 120 and 34 which are defined by both the fuel distribution assembly 110, and the support member 20. Further, a plurality of fastener apertures 126 are formed in the individual seals 123, and 124, and are operable to receive fasteners which will be discussed in greater detail hereinafter.

Lying in immediate juxtaposed relation relative to the second pressure sensitive adhesive seal 124 is an anode current collector which is generally designated by the numeral 140. Additionally, and as seen in FIG. 7, a substantially rigid sealing plate 130 is provided and which is juxtaposed relative to the cathode side 102 of the membrane diffusion assembly 100. The sealing plate 130 has a main body 131 which defines a plurality of apertures 132 which matingly receive, in part, the respective membrane electrode diffusion assemblies 100. Still further, the main body has a plurality of fastener apertures 133 formed therein and which when assembled, are substantially coaxially aligned with the aforementioned fastener apertures formed in the earlier described portions of the fuel cell module 11.

Each anode current collector 140 lies in ohmic electrical contact against the anode side 101 of each of the membrane electrode diffusion assemblies 100 and further is oriented in heat receiving relation relative thereto. The anode current collector 140 has an electrically conductive main body 141 which has an inside facing surface 142 which lies against the anode side 101 of the membrane electrode diffusion assembly 100, and an opposite outside facing surface 143. Still further, a plurality of fastener apertures 144 are formed in the main body 131 and are operable to be substantially coaxially aligned relative to the other fastener apertures 126 formed in the various seals 123,124, and in the fuel distribution assembly 110.

As seen in FIG. 7, an electrically insulative member or gasket 160 is mounted or oriented in juxtaposed relation relative to the outside facing surface 143 of the anode current collector 140. This insulative member has a main body 161 which has an inside facing surface 162 which rests in contact with the outside facing surface 143 of the anode current collector, and further has an outside facing surface 163. Further, a plurality of fastener apertures 164 are operable to be coaxially aligned with the previously described fastener apertures formed in the remaining parts of the ion exchange membrane fuel cell power system 10.

As best seen in FIG. 7, an anode heat sink 170 is oriented in juxtaposed relation relative to the insulative member 160, and further, is mounted in heat receiving relation relative to the anode sides 101 of each of the membrane electrode diffusion assemblies 100 to conduct heat energy generated by the ion exchange membrane module 11 away from the membrane electrode diffusion assembly 100. In this arrangement, the fuel distribution assembly 110 is located substantially between the anode side 101 of the membrane electrode diffusion assembly 100, and the anode current collector 140. The anode heat sink 170 has a main body 171 which has an inside facing surface 172, which lies in juxtaposed relation relative to the insulative member 160, and an opposite outside facing surface 173. Similarly, and as discussed above, numerous fastener apertures 174 are formed therein, and which are substantially coaxially aligned with the remaining fastener apertures which are formed in the earlier disclosed portions of the ion exchange membrane fuel cell module 11. Fasteners 175 are provided and are received in these coaxially aligned fastener apertures such that the module is held firmly together. These fasteners 175 along with the respective current collectors 60 create sufficient pressure to allow the individual current collectors 60 and 140 to make effective ohmic electrical contact with the anode and cathode sides 101 and 102, respectively, of the membrane electrode diffusion assembly 100. As will be recognized from the discussion above, the anode current collector 140 is substantially electrically isolated from the anode heat sink 170. Additionally, the anode heat sink has sufficient thermal conductivity such that it substantially inhibits the formation of a temperature gradient across the membrane electrode diffusion assembly 100 during operation of the ion exchange membrane fuel cell power system 10.

A handle assembly is generally indicated by the numeral 190 and is best seen in FIG. 2. As shown therein, the handle assembly 190 has a back plate generally indicated by the numeral 191, and which is defined by a front surface 192, and an opposite rear surface 193. Formed through the front and rear surfaces is an aperture 194 which matingly receives the member 84 which is mounted on the main body 81 of the current conductor assembly 80. Still further, a pair of handles 195 are fastened on the front surface 192, and additionally, a plurality of fastening apertures 196 are formed through the front and rear surfaces 192 and 193 and are operable to receive fasteners 197 which threadably engage the fastener apertures 31, which are formed in the forward edge 23 of the support member 20. The handles permit the module 11 to be easily manipulated by hand, and removed without the use of any tools, when utilized with a subrack or sub-system which will be discussed in greater detail hereinafter.

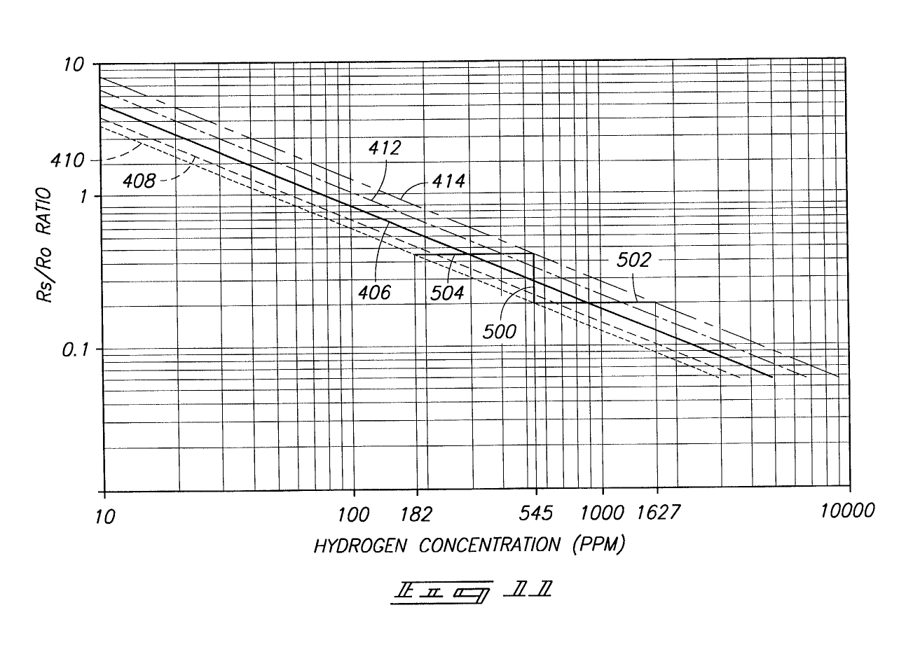

The ion exchange membrane fuel cell module 11 is employed in combination with a plurality of subracks or sub-systems 210, one of which is shown in FIGS. 8 and 9 and which is generally indicated by the numeral 210. Each subrack 210 releasably supports a plurality of ion exchange membrane fuel cell modules 11 in an operable arrangement. Each subrack 210 includes a principal enclosure 211. The principal enclosure is defined by a top surface 212; bottom surface 213; front sidewall 214; rear sidewall 215; left sidewall 216, and right sidewall 217. The respective sidewalls 212 through 217 define an internal cavity 220 (FIG. 9). In this arrangement, the principal enclosure will receive multiple fuel cell modules 11, each enclosing a membrane electrode diffusion assembly 100.

As seen in FIG. 8, the ion exchange membrane fuel cell power system is configured in a manner where at least one of the fuel cell modules 11 can be easily removed from at least one of the subracks 210 by hand, while the remaining modules continue to operate. As noted above this removal is normally accomplished without the use of any tools, however it may be necessary in some commercial or industrial applications where vibration, and other outside physical forces may be imparted to the system, to use threaded fasteners and the like to releasably secure the individual modules to the subrack 210 to prevent the unintentional displacement or dislocation of the respective modules from the subrack 210. If utilized, the hand tools which will be employed will be simple hand tools, and the removal will be accomplished in minutes, as opposed the prior art stack arrangements where replacement of a damaged membrane electrode assembly (MEA) may take hours to accomplish. It should be understood that the terms “subrack” and “sub-system” as used in the following claims do not necessarily imply that a rack or shelf is required, only that the sub-system, or a portion thereof, is operable independently whether or not other sub-system, or a portion thereof, of the fuel cell power system 10 are functioning.

As best seen by reference to FIG. 9, an aperture 230 is formed in the top surface 12 of the subrack 210, and further, the cavity 220 is comprised of a first or fuel cell module cavity 231, and a second cavity or electrical control bay 232. As best seen by reference to FIG. 8, a plurality of individual module apertures 233 are formed in the front surface 214 of the principal housing 211, and are operable to individually receive the respective fuel cell modules 11, and position them in predetermined spaced relation, one to the other.

The fuel cell module cavity 231 is further defined by a supporting member or shelf 234 (FIG. 9) which orients the individual fuel cell modules 11 in a predetermined substantially upright orientation within the cavity 231. Additionally, the fuel cell module cavity 231 is defined by a rear wall 235 which supports a DC bus 236 in an orientation which will allow it to releasably, matingly, electrically couple with the current conductor assembly 80 (FIG. 2) which is borne by the fuel cell module 11. Yet further, and as seen in the cross sectional view of FIG. 9, the rear wall 235 further supports a fuel supply line 237 and a byproduct removal line 238. These are operable to be releasably coupled in fluid flowing relation with respect to the fuel delivery passageway 41 and the exhaust passageway 44 of the fuel coupling 40.

As best seen in FIG. 9, the second cavity or electrical control bay 232 encloses a digital or analog controller 250 which is electrically coupled with the respective ion exchange membrane fuel cell modules 11, and a power conditioning assembly 260 which is electrically coupled with the DC bus 236, and the controller 250, and which is operable to receive the electrical power produced by the ion exchange membrane fuel cell modules 11. The operation of the controller 250 and power conditioning assembly 260 and related control circuitry is discussed in prior U.S. application Ser. Nos. 09/108,667 and 09/322,666, which are incorporated by reference herein.

As further seen in FIG. 9, an aperture 270 is formed in the rear wall 215 of the principal enclosure 211, and is operable to receive an air filter 271 which is operable to remove particulate matter from an outside ambient air stream passing therethrough and into the principal enclosure 211.

As best seen by the cross sectional view in FIG. 9, the subrack 210 includes an air distribution plenum 290 which is coupled in fluid flowing relation relative to each of the ion exchange membrane fuel cell modules 11.

The air distribution plenum 290 has a first or intake end 291 which receives both air which has previously come into contact with each of the ion exchange fuel cell modules 11, and air which comes from outside of the respective ion exchange membrane fuel cell modules. Further, the air distribution plenum has a second or exhaust end 292 which delivers an air stream to each of the ion exchange fuel cell modules 11. Disposed intermediate the first or intake end 291, and the second or exhaust end 292 is an air mixing valve 293 which is coupled to the air distribution plenum 290, and which meters the amount of air which is passed through the respective ion exchange membrane fuel cell modules 11 and is recirculated back to the ion exchange fuel cell membrane modules and by way of the air filter 271.

As illustrated, the mixing valve 293 selectively occludes an aperture 294 which is formed in the rear wall 215 of the subrack 210.

An air movement assembly such as a fan 295 is provided and is mounted along the air distribution plenum 290. As shown in FIG. 9, the air movement assembly 295 is positioned near the intake end 291, and is substantially coaxially aligned with the aperture 230 which is formed in the top surface 212 of the subrack 210. The air mixing valve and the fan assembly 293 and 295 respectively are electrically coupled with the controller 250 and are controlled thereby. The air mixing valve 293 comprises a pivotally movable valve member 296 which can be moved from a first occluding position 297 relative to the aperture 294, and a second, substantially non-occluding position 298 as shown in phantom lines.

As will be recognized, when the valve member 296 is in the second non-occluding position, air received in the intake end 291 and which has previously passed through the individual fuel cell modules will pass out of the principal enclosure 211 and then be exhausted to the ambient environment. On the other hand, when the valve member 296 is in the occluding position 297 air from the intake end 291 which has passed through the fuel cell module 11 will return to the exhaust end and then pass through the modules 11 and return again to the intake end. As will be recognized, by controlling the relative position of the valve member 296, temperature as well as relative humidity of air stream 299 can be easily controlled. Still further, in the occluding position 297, air from the ambient will continue to enter the air distribution plenum by way of the air filter 270.

More specifically, the air stream 299 which is supplied to the fuel cell modules is provided in an amount of at least about 5 to about 1000 times the volume required to support a fuel cell chemical relation which produces water vapor as a byproduct. The present air plenum arrangement provides a convenient way by which the air stream delivered to the cathode side 102 can be humidified by the water vapor generated as a byproduct of the chemical reaction taking place on the cathode. Additionally, during cold operating conditions, this same air, which has now been heated by each of the fuel cell modules 11, will contribute to bringing the entire fuel cell up to normal operating temperatures. Further, the air mixing valve 293 limits the amount of air which has previously passed through the modules 11 and which is added to the air distribution plenum 290. This resulting recirculated air stream and fresh ambient air forms an air stream having substantially optimal operating characteristics which maximizes the current densities and outputs of the respective membrane electrode diffusion assemblies enclosed within each of the fuel cell modules 11.

Referring now to FIG. 10, what is shown is a greatly simplified, exaggerated, partial, and cross-sectional view of an ion exchange membrane fuel cell module 11 positioned in an operational relationship relative to the air distribution plenum 290. This particular sectional view, which does not include many of the subassemblies previously discussed, is provided to illustrate the principals that will be set forth below. As seen in FIGS. 9 and 10, and as discussed above, the subrack 210 includes an air distribution plenum 290 which provides a stream of air 299 to each of the ion exchange fuel cell modules 11 which are received in an operational position on the shelf or supporting member 234. The air stream 299 exits from the exhaust end 292 and then becomes a bifurcated air flow which is generally indicated by the numeral 320. The bifurcated air flow 322 comprises a first cathode air stream 321, which is received in the respective ion exchange membrane fuel cell modules 11; and a second anode heat sink air stream which is generally indicated by the numeral 322. As will be recognized by a study of FIG. 10, the first cathode air stream 321 enters the ion exchange membrane fuel cell module 11, and is further bifurcated into a first component 323 which moves along one of the cathode air passageways 106 which is defined on one side of the support member 20. Further, the first cathode air stream 321 has a second component 324 which passes along the cathode air passageway 106 on the opposite side of the support member 20. As will be appreciated, the bifurcated cathode air stream 321 provides the necessary oxidant (oxygen in the ambient air stream) to the cathode side 102 of the membrane electrode diffusion assembly 100. Yet further, the cathode air flow operates to remove less than a preponderance of the heat energy generated by the membrane electrode diffusion assembly 100 while it is in operation. As will be recognized the cathode air flow is facilitated by the respective cathode current collectors 60 which create in part, the cathode air passageway 106.

The anode heat sink air stream 322 is further bifurcated into a first component 325 and a second component 326, both of which individually move along the opposite sides 16 of the ion exchange membrane fuel cell module 11, and over each of the anode heat sinks 170. As the anode heat sink air stream components 325 and 326 move over the opposite anode heat sinks 170, the anode heat sink air stream operates to remove a preponderance of the heat energy generated by the ion exchange membrane fuel cell module 11 during operation. Therefore, it will be recognized that the present invention provides an ion exchange fuel cell module 11 which has a bifurcated air flow 320 which regulates the operational temperature of the ion exchange membrane fuel cell module by removing the heat energy generated therefrom.

Referring now to FIG. 8, and as earlier discussed, the individual ion exchange membrane fuel cell modules 11 and the subrack 210 comprise in combination a fuel cell power system which is coupled in fluid flowing relation relative to a source of a substantially pure or dilute fuel generally indicated by the numeral 340. The fuel gas supply may comprise a source of bottled and compressed fuel gas generally indicated by the numeral 341, or a fuel stream which is provided by a chemical reactor, or fuel processor 342 which produces the fuel stream for use by the individual ion exchange fuel cell modules 11. A conduit 343 couples either fuel gas supply 340 with the respective ion exchange fuel cell modules 11 and the associated subrack 210. When a chemical fuel processor 342 is provided, the fuel processor would receive a suitable hydrocarbon fuel stream such as natural gas, propane, butane, and other fuel gases and would thereafter, through a chemical reaction release a fuel stream which would then be delivered by way of the conduits 343.

The present fuel cell power system 10 may also include a fuel gas recovery and recycling system (not shown) which would recover or recapture unreacted fuel gas which has previously passed through the individual ion exchange fuel cell modules 11. This system, in summary, would separate the unreacted fuel gas and would return the unreacted fuel gas back to the individual ion exchange fuel cell modules for further use. This recovery system would be coupled with the byproduct removal line 238.

Although a certain number of subracks 210 are shown in the drawings, and a certain number of fuel cell modules 11 are shown per subrack 210, it will be readily apparent that any desired number of subracks and modules 11, or a portion thereof, could be employed in alternative embodiments.

The fuel cell power system 10 (FIG. 9) includes one or more gas sensors 400 in one or more locations and which are used, for example, to detect the presence of fuel (e.g., hydrogen gas). The presence of hydrogen gas in certain areas of the fuel cell power system 10 of the subracks 210 may indicate a fuel leak. Such fuel leaks can be potentially hazardous under certain operating conditions. One such sensor 400 is shown in FIG. 9. The sensor 400 has a sampling port 403, including a sensor element, and a baffle protecting the sensor element; e.g., from high velocity airflow. The sampling port 403 is the part of the sensor primarily exposed to the target gas. In one embodiment, the baffle comprises a sintered bronze disk. Other alternatives could be employed. For example, the baffle could just as easily be a piece of chemist's filter paper. Further, if the sampling port 403 is located in an area that does not have ventilation or high airflow, the baffle is not necessary and can be omitted altogether.

The sensor 400 includes a heater for heating the sensor element to a predetermined operating temperature. The heater can be, for example, a wire that is spirally wound relative to the sensor element. Such heaters provide heat in a predefined temperature range to assure proper operation of the accompanying sensor. Other configurations are, of course, possible for the sensor 400. In operation, an electrical current is applied to the heater associated with the sensor 400, at a predetermined power level, to maintain the element at a specified operational temperature. For example, with one commercially available sensor, approximately 600 mW of power maintains the sensor at a temperature of 500° C.

The fuel cell power system 10 further includes circuitry 402 which is electrically coupled to the sensor 400. The circuitry 402 controls operation of the sensor 400 (e.g. generation of heat by the heater included in the sensor 400) and further is coupled to the controller 250. In one embodiment, the circuitry 402 is a printed circuit card associated with the sensor 400 and which is provided by the manufacturer thereof.

In one embodiment, for example (see FIG. 9), the gas sensor 400 is positioned such that it may sense hydrogen gas in the plenum 290. In this embodiment, the gas sensor 400 is primarily housed in the cavity or electrical control bay 232. The circuitry 402 associated with the card (discussed above) is also located in the electrical control bay 232 and is mounted, for example, on 1/4-inch-long standoffs which are affixed to the top of the control bay 232. As seen in FIG. 9, the sampling port 403 protrudes through the bulkhead 405 separating the chamber 232 and the plenum 290 in order to position the sampling port 403 inside plenum 290.

Other locations for the sensor 400 are, of course, possible. The location is, in the illustrated embodiment, selected such that the sampling port 403 is positioned downstream of the fan 295. This location insures that leaking hydrogen is homogenized into the air, but is detected before encountering any mixing vanes 293, or where fresh air is introduced 271. Further, the location, in the illustrated embodiment, is selected such that the circuitry 402 and the electrical connector between the circuitry 402 and the sensor 400 are located within the control bay 232 so that this connector does not have to pierce the bulkhead 405. This also allows the electronics of the circuitry 402 to be located in an area that is cooled via fan-forced air.

Alternatively, the sensor 400 may be located in the plenum 290 and the electrical connector between the circuitry 402 and the sensor 400 must then pierce the bulkhead 405. This is less desirable because the fuel cell system 10 circulates air at about 55° C. and this higher temperature lessens the life of power-producing electronic components on the circuitry 402.

Further, a seal is required where the cable pierces the bulkhead 405.

The fuel cell power system 10 further includes dew point determining equipment 401. In one embodiment of the invention, the dew point determining equipment comprises chilled-mirror equipment, configured to provide a signal representative of the dew point. Chilled-mirror dew point determining equipment is described, in greater detail, in the following U.S. patents which are incorporated herein by reference: U.S. Pat. Nos. 5,739,416 to Hoenk; 5,507,175 to Cooper; and 6,155,098 to Shapiro et al. In an alternative embodiment, the dew point determining equipment comprises a temperature sensor and a relative humidity sensor.

In the present embodiment, the circuitry 402 is coupled to the dew point determining equipment 401 as well as to the sensor 400. In this embodiment, the circuitry 402 compensates the sensor 400 for the effects of dew point. Alternatively, the compensation can be performed elsewhere, such as in the controller 250.

While other sensors could be employed, in the illustrated embodiment, the sensor 400 is a metal oxide semiconductor (MOS) hydrogen sensor, model TGS 821, and which is commercially available from Figaro Engineering (Figaro). Figaro's sensors are described in the following U.S. patents, which are incorporated herein by reference: U.S. Pat. Nos. 5,006,828 to Yutaka et al.; 4,958,513 to Yasunga et al.; 4,938,928 to Koda et al.; 4,827,154 to Naoyuki et al.; 4,816,800 to Onaga et al.; 4,731,226 to Takahata et al.; 4,718,991 to Yamazoe et al.; 4,701,739 to Sasaki; 4,658,632 to Sasaki; 4,575,441 to Murakami et al.; 4,459,577 to Murakami et al.; and 4,117,082 to Matsuyama.

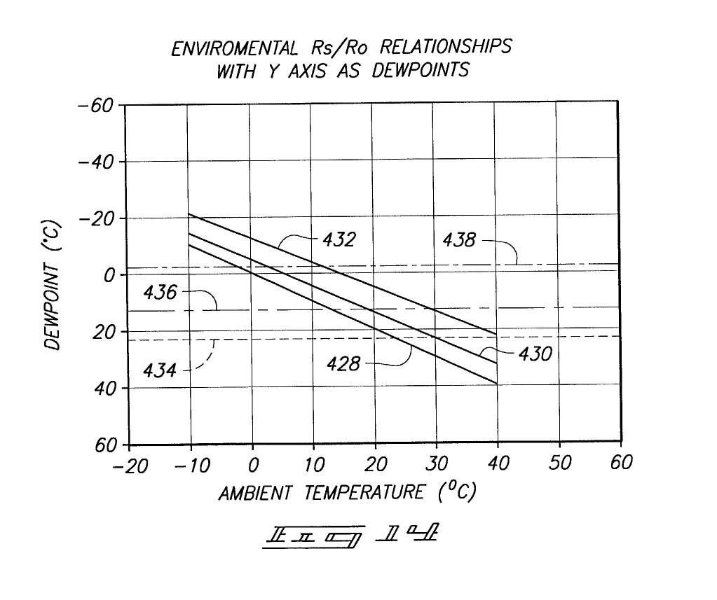

FIG. 11 illustrates temperature/humidity dependency. More particularly, FIG. 11 shows the average ratiometric response of one sensor model, the Figaro TGS821, to hydrogen and shows environmental offsets. The relationship between hydrogen concentration versus relative resistance, at an environmental dependency Rs/Ro of unity (where the effects due to temperature and relative humidity are null), can be described by a formula of the form Y=MX+B and is given by the function Y=10ˆ (αlog(W)−2α). Here, “Y” is the Rs/Ro ratio and is the sensor's sensitivity ratio normalized to unity at a gas concentration of 100 PPM. The term “W” is the hydrogen gas concentration in PPM. The term “α” (alpha) describes the sensor's sensitivity slope (how steep it is). Whereas a for one particular sensor (Figaro TGS821) averages −0.725, manufacturing tolerances are such that a ranges, for example, from −0.6 to −1.2. The actual sensor resistance for any given hydrogen concentration at an environmental dependency Rs/Ro of unity, is given by the formula R=10ˆ (3.5±0.5)Y The term “R” is sensor resistance (Ω) and the term “Y” is the sensor's sensitivity Rs/Ro ratio. This means that at an environmental dependency Rs/Ro ratio of unity, the average sensor of this model has about 3.2kΩ of resistance at 100 PPM, but ranges from 1.0 kΩ to 10 kΩ. As can be seen, the sensitivity of the sensor defined by the relationship between gas concentration changes and the sensor resistance changes is based on a logarithmic function. The x-axis is gas concentration and the y-axis is indicated as a sensor resistance ratio Rs/Ro where Rs is sensor resistance. In the graph of FIG. 11, the four slopes 408, 410, 412, 414 adjacent to the main (bold) one 406 denote the extent to which temperature and relative humidity—environmental dependencies—can affect the sensor's signal output in fuel cell applications. Slope 406 is an environmental dependency Rs/Ro of unity, slope 408 is an environmental dependency Rs/Ro of 0.8, slope 410 is an environmental dependency Rs/Ro of 0.6835, slope 412 is an environmental dependency Rs/Ro of 1.25, and slope 414 is an environmental dependency Rs/Ro of 1.5109. Whereas a averages −0.725, it can range, for example, from −0.6 to −1.2.

Note the error bar 500 on FIG. 11. Without circuitry to compensate for the environmental effects fuel cells are subject to or knowledge of the environmental circumstances, a reported reading of 545 PPM could reside anywhere on the line segment 502 projecting to the right from the bottom of the error bar (at an Rs/Ro ratio of 0.200) and the true concentration could be as great as 1627 PPM. Just as easily, a reported reading of 545 PPM could reside anywhere on the line segment 504 projecting to the left from the top of the error bar (at an Rs/Ro ratio of 0.442) and the true concentration could be as little as 182 PPM. This is an 8.9:1 range of uncertainty and is the source of much frustration with uncompensated MOS gas sensors. It may also be advantageous to have circuitry associated with a sensor, such as circuitry 402 in the present embodiment, be of an all-analog design (i.e., a design with no microprocessor at the heart of the device continually running firmware or software) in certain embodiments.

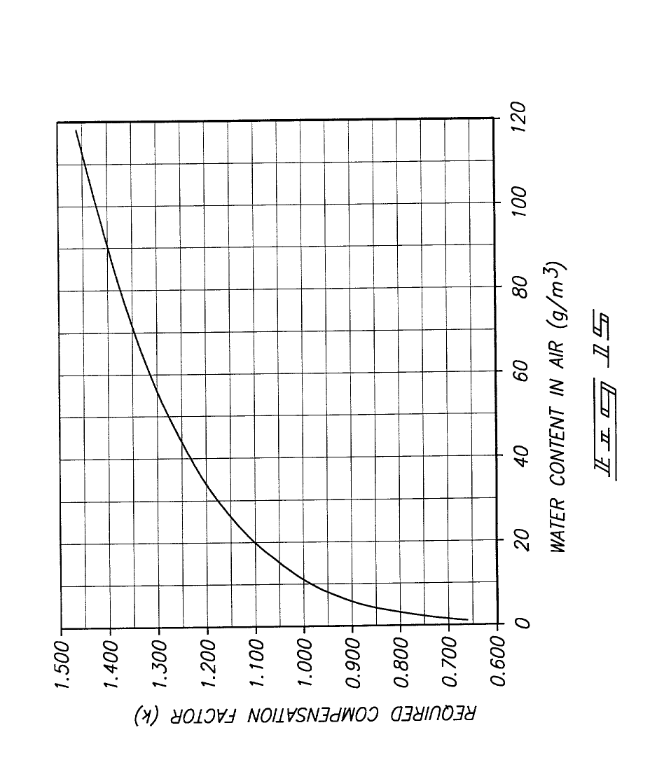

FIG. 12 provides an indication of the conventional way manufacturers of MOS gas sensors look at the effects of temperature and relative humidity. The solid lines 416, 418, and 420 are relative humidities of 95%, 65%, and 35% respectively. FIG. 12 illustrates the conventional view that MOS sensors' Rs/Ro ratios (environmental dependencies) along the y-axis are functions of an infinite number of combinations of relative humidities and ambient temperatures. Therefore, the conventional approach to compensating for environmental dependencies is to use a microprocessor and digital lookup charts to compensate separately for these influences. This method is only an approximation—particularly at lower temperatures and relative humidities.

Experiments conducted by the inventors have led them to discover that the effects of temperature and relative humidity on metal oxide semiconductor MOS gas sensors can be reduced to the single variable of dew point. In this regard it has also been found that dew point compensation is applicable to sensors other than hydrogen sensors. Dew points are consistent at each Rs/Ro ratio for various types of sensors. Thus, though FIG. 9 shows the use of hydrogen sensors, the present invention has application to other types of sensors which may or may not be used in a fuel cell embodiment to detect the presence of hydrogen or another fuel gas.

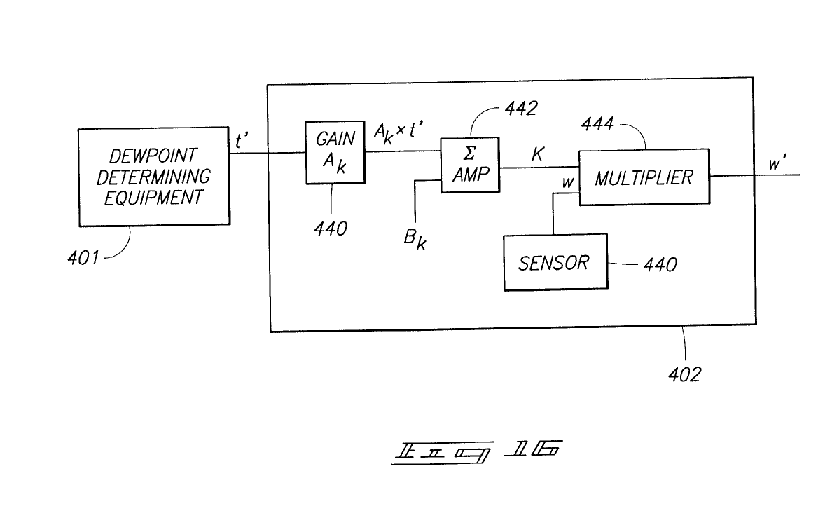

The environmental uncertainties shown in FIG. 12 are caused by variations in the air's water content as will later be shown in connection with FIGS. 13-15. Manufacturer data includes multiple distinct slopes 416, 418, and 420 of interacting temperatures and relative humidities. Line 416 indicates temperature/humidity dependency at 95 percent relative humidity, line 418 indicates temperature/humidity dependency at 65 percent relative humidity, and line 420 indicates temperature/humidity dependency at 35 percent relative humidity for a particular model sensor, namely the Figaro 821. The FIG. 12 data indicates the sensor's dependencies to temperature and relative humidity according to the manufacturer of same.

FIG. 13 takes the average dew points found at various Rs/Ro values from manufacturer-supplied data of FIG. 12 and plots them against the reciprocal of the Rs/Ro values. As can be seen from FIG. 13, a simple linear function produces an excellent fit to the data points. As seen, the reciprocal of the Rs/Ro values were plotted. If a certain dew point produces an environmental Rs/Ro value of 0.800, an appropriate compensation factor (K) for this model of sensor would be 1.25 (1/0.8).

Referring back to FIG. 12, the dashed lines 422, 424, and 426 in FIG. 12 are of fixed relative humidity, but they relate dew point (y-axis) to temperature (x-axis). Dashed lines in FIG. 12 are supplied by applicant, not by the manufacturer of the previously mentioned sensors. The close relationship of these dashed lines to the manufacturer supplied lines 416, 418, and 420, that separately consider temperature and relative humidity, can be seen in FIG. 12.

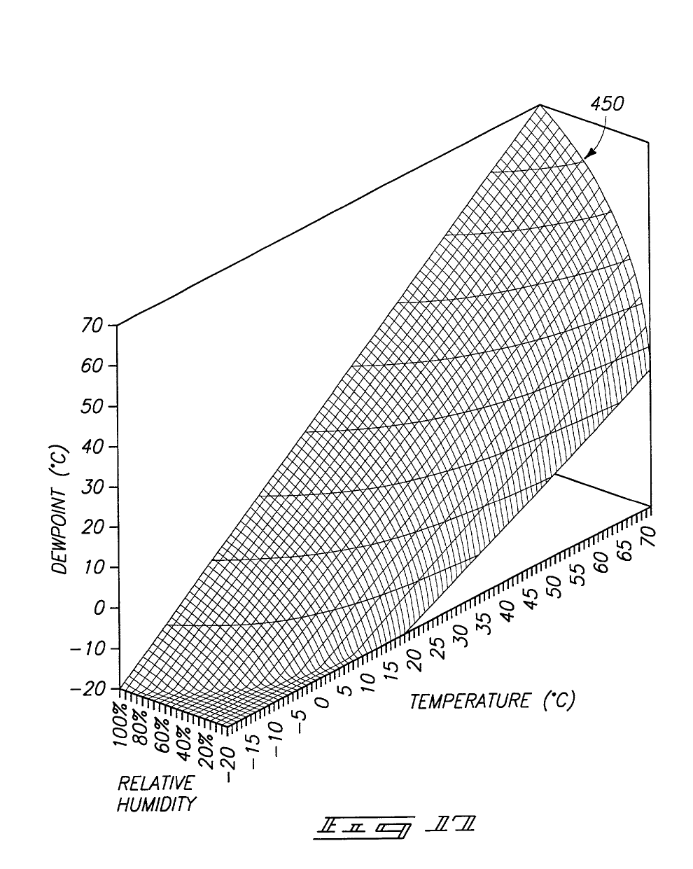

FIG. 14 is a variation of the graph shown in FIG. 12. In FIG. 14, the Rs/Ro ratios on the y-axis have been replaced with dew points. Slope 428 represents a relative humidity of 95 percent, slope 430 represents a relative humidity of 65 percent, and slope 432 represents a relative humidity of 35 percent. For reference, three of the manufacturer's Rs/Ro ratios—0.9, 1.0, and 1.2—are superimposed over the data. Line 434 represents a Rs/Ro ratio of 0.9, line 436 represents a Rs/Ro of 1.0, and line 438 represents a Rs/Ro of 1.2.

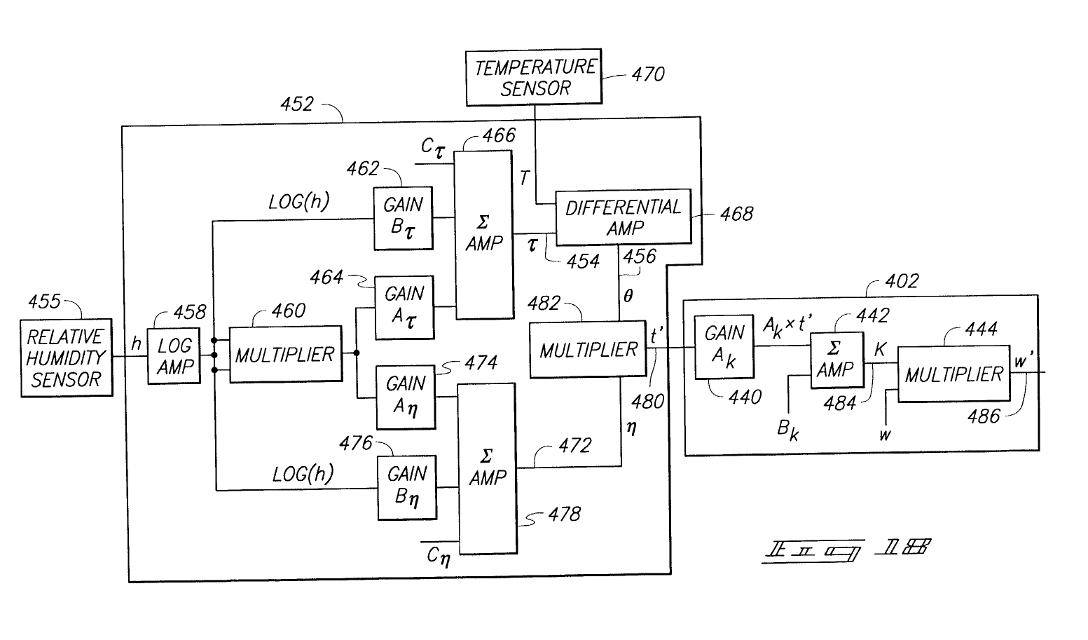

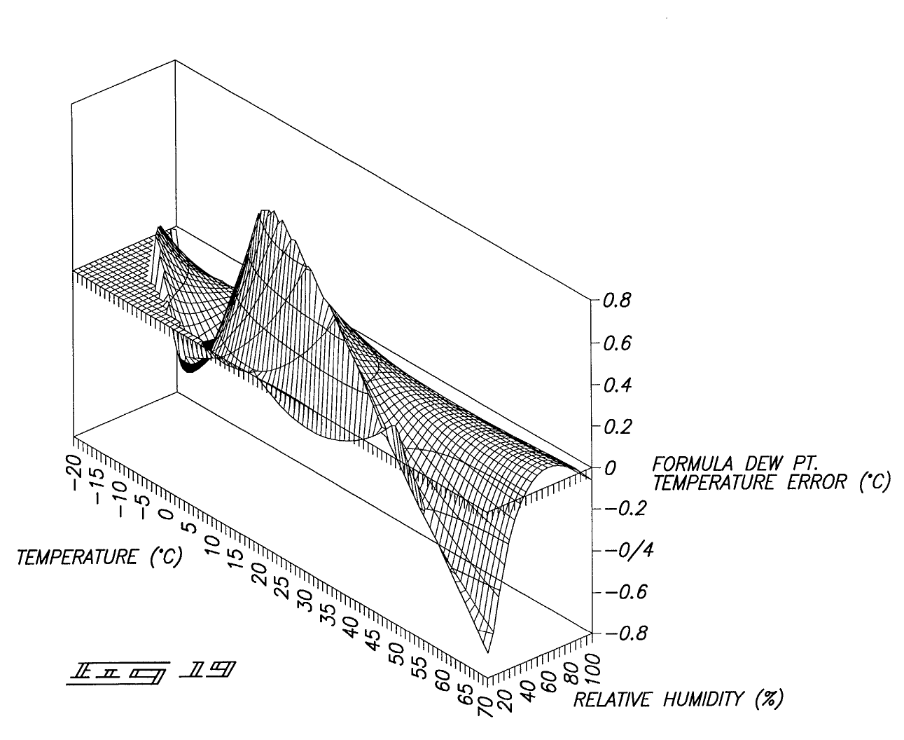

FIG. 15 shows the relationship of compensation factor vs. mass content of water in air, the industry's metric. When the industry calculates compensation factors, it measures water content in terms of grams per cubic meter. This obscures the relationship between Rs/Ro environmental dependencies and dew point because the relationship to g/m3 produces a nearly straight line only when viewed on a log/linear graph. As shown in FIGS. 13-14, measuring water content in terms of dew point is a much more straightforward endeavor.

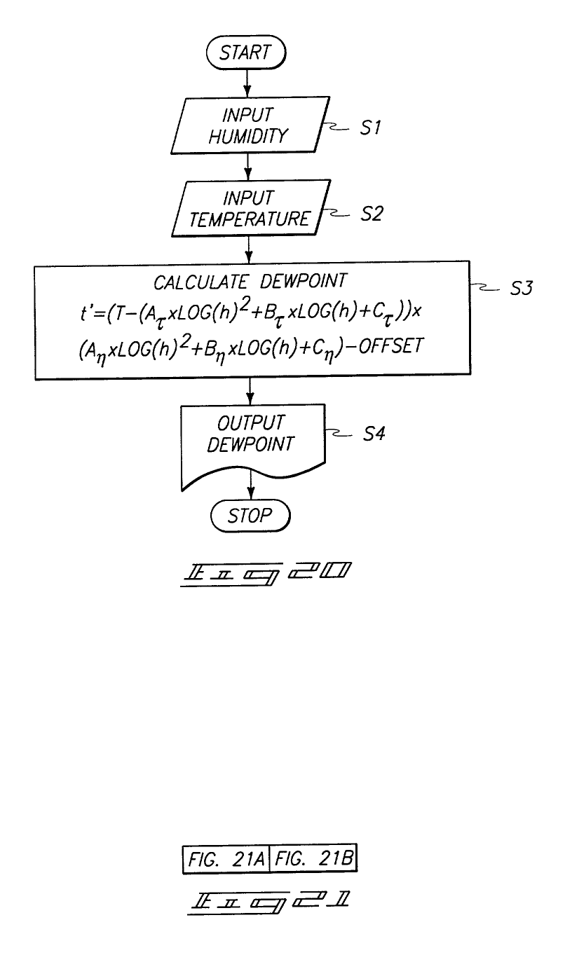

Therefore, in operation, a method of compensating MOS gas sensor 400 in accordance with one aspect of the present invention comprises using MOS gas sensor 400 to provide a signal indicative of gas concentration of a target gas (e.g., hydrogen) in an ambient (e.g., in the plenum 290); providing a signal representative of dew point of the ambient; and modifying the signal from the MOS gas sensor 400 using the signal representative of dew point to simultaneously compensate for the effects of both temperature and relative humidity. The signal from the gas sensor 400 is modified by the conditioning circuitry 402. In one embodiment, the circuitry 402 comprises analog circuitry. The signal from the gas sensor is modified by the circuitry 402 using the signal representative of dew point by transforming the dew point signal into a linear or semi-linear compensation factor and multiplying that compensation factor by the output of the sensor 400 (see FIG. 16). For one model sensor, the Figaro TGS821 hydrogen sensor, the best fit for sensors with an average a of −0.725 is a simple y=ax+b linear transformation, which is performed as follows: K=(aₖ×t′)+bₖ, where K is the dew point compensation factor (0.6619≦K≦1.463); t′ is dew point in ° C., aₖ=0.0109, and bₖ=0.86352. K is a compensation factor between 0.6619 (−18.5° C. dew point) and 1.463 (55° C. dew point). This range of compensation factors will compensate for environmental Rs/Ro values ranging from 1.511 (the reciprocal of 0.6619) through 0.6835 which is the lowest expected value for the fuel cell system 10 of the illustrated embodiment. In the preferred embodiment, the above-described aₖ and bₖ coefficients are fixed constants of 0.0109 and 0.86352 respectively and the signal representing the response slope of the sensor (which can range from −0.6 to −1.2) is normalized during calibration to an α of −0.725 before being compensated for the effects of dew point. This is because the magnitude of required change in a particular sensor's signal due to dew point is proportional to the magnitude of its response to hydrogen.

In an alternative embodiment, it is not necessary to normalize the sensor's gain to a specific alpha (such as −0.725) during calibration. This can be accomplished by instead scaling the influence of K factor depending on alpha. For alphas between −1.200 and −0.725, the effect of K factor is accentuated, and for alphas between −0.600 and −0.725, the effect of K factor is attenuated. This is accomplished with the following function: Rcomp=10ˆ (log(Rs)+(log(K)/α₀)α). Here, “Rs” is the signal coming from the MOS sensor in terms of resistance (Ω). The term “α₀” is the alpha value at which K factors were optimized for, in this case, where aₖ=0.0109 and bₖ=0.86352 are the terms used for converting dew point into K factors, α₀=−0.725. The term “α” is the alpha for that particular sensor. The term “Rcomp” is the sensor resistance (Ω) with the effects of environmental dependencies nulled. Hydrogen concentration is then generated with a circuit (or software) that performs the following function: w′=((log(Rcomp/Ro)+2α)/α). Here, the term w′ is the compensated hydrogen concentration (PPM). “Ro” is the resistance (Ω) for that particular sensor at 100 PPM at a K factor of unity. For the TGS821, Ro is typically about 3.2 kΩ but can range from 1.0 kΩ to 10 kΩ.

In still another alternative embodiment, the sensor's a need not be normalized to −0.725 during calibration and its response signal may be directly acted on by the dew point compensating circuitry. In one such embodiment, the coefficients aₖ and bₖ are not constants and are instead variables that are direct functions of alpha. Alternatively, improved accuracy across the full range of a can be achieved by converting t′ into K using a second-order polynomial transformation where the three coefficients aₖ, bₖ and cₖ are variables that are direct functions of alpha. Other alternatives are, of course, possible.

Thus, the circuitry 402 includes (see FIG. 16) an amplifier 440 which, in operation, multiplies t′ by aₖ, a summing amplifier or adder 442 which, in operation, adds that product to bₖ to produce the dew point compensation factor K, and a multiplier 444 which, in operation, multiplies the dew point compensation factor K by the output of the sensor 400. In one alternative embodiment, the functionality of the circuitry 402 is implemented in digital circuitry instead of the illustrated analog circuitry. In another alternative embodiment, the functionality of the circuitry 402 is implemented in the controller 250.

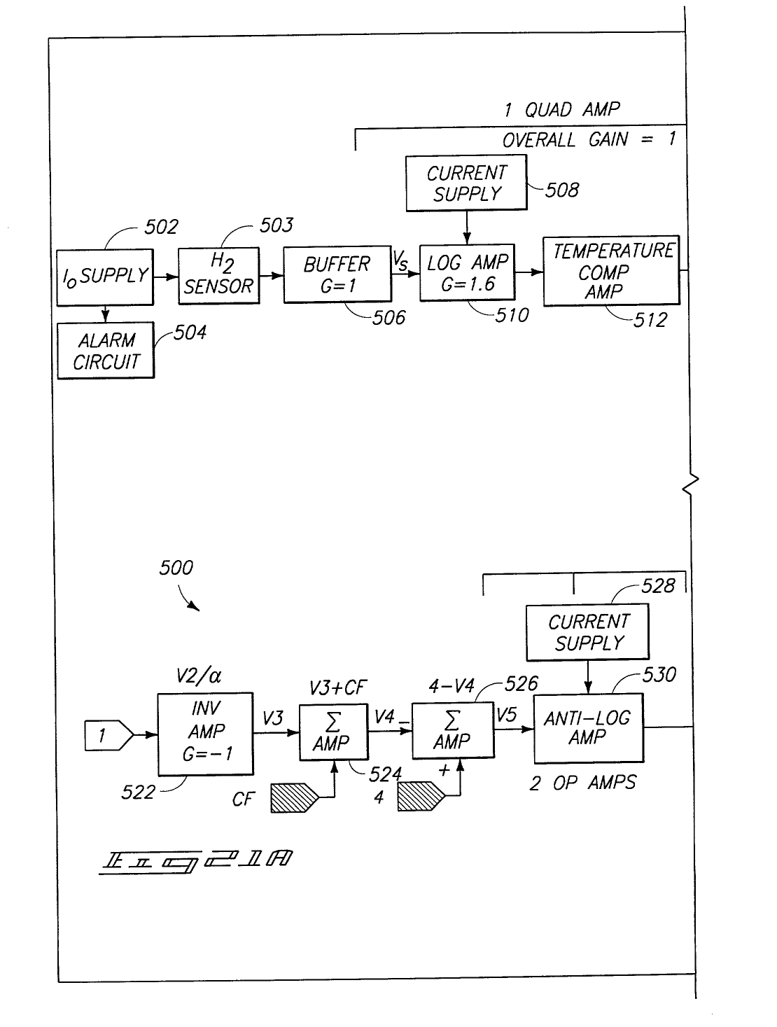

FIG. 17 illustrates a dew point “sail” 450 Note how the sail intersects the floor of the graph and how the shape of this intersection forms a curve. This curve describes the relationship between relative humidity and temperature at a dew point of −20° C. This curve can also be described by a node of an analog circuit or by an intermediate result in a digital implementation.

Along the top edge of the sail of FIG. 17, against the 100% relative humidity wall, for each degree the temperature rises, the dew point also rises by one degree. The slope at this edge (100% relative humidity) is therefore 1:1. The slope at the lower edge, at 5% relative humidity, is 0.632:1. The way the slopes transition from 1:1 to 0.632:1 as relative humidity changes can also be described by a node of an analog circuit or by an intermediate result in a digital implementation.

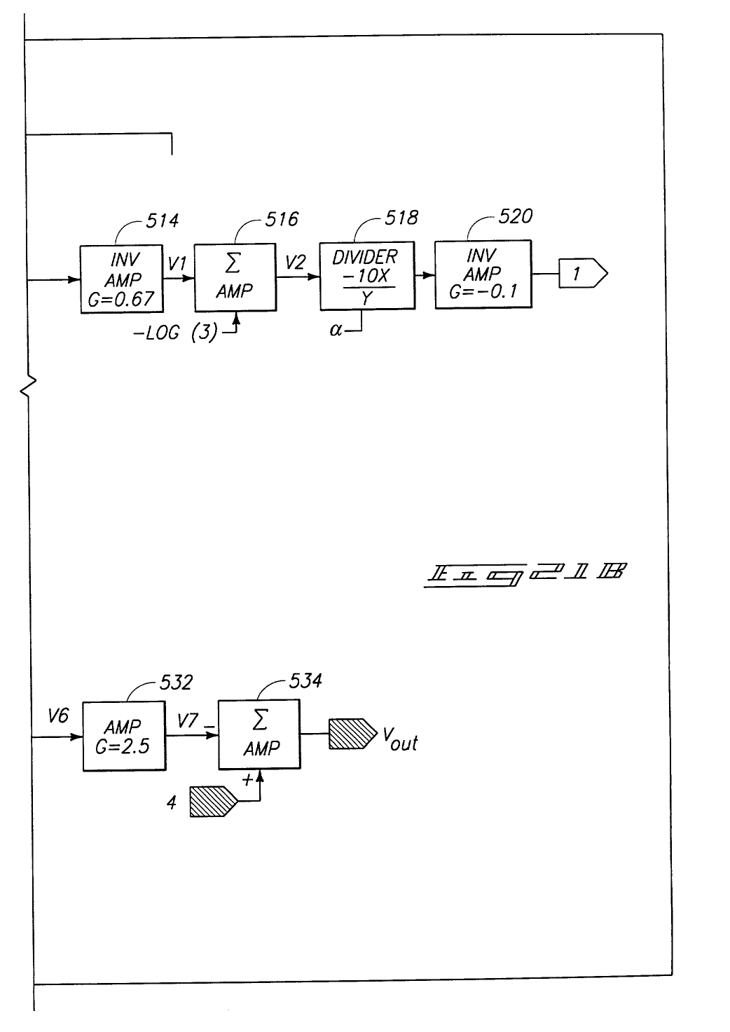

Using such analysis, the inventors have designed analog circuitry 452 (FIG. 18) capable of outputting dew point when given the inputs of relative humidity and temperature. FIG. 18 illustrates nodes which describes intermediate signals or calculations as will be described below.:

The temperature signal τ that correlates to a dew point of −20° C. is provided at a node 454 and is determined using a humidity signal H from, for example, a humidity sensor 455. The signal H is converted into the temperature signal that correlates to a particular dew point, −20° C. in the illustrated embodiment. This is determined by finding the temperature on the base intersection curve in FIG. 17. This is performed by considering the following equation:

τ=aτlog(H)ˆ 2+bτlog(H)+Cτ

where H is the relative humidity signal in full percentage counts, such as “50” for 50% relative humidity (1≦H≦100); aτ=5.65; bτ=−44.3; cτ=66.14; and τ=temperature, in Kelvins beyond a −20° C. baseline, at which H correlates to a dew point of −20° C. (0.14≦τ≦66.14).

More particularly, the circuitry 452 includes a logarithmic amplifier 458 having an output, and having an input coupled to the humidity sensor 455. The circuitry 452 further includes a multiplier 462 having an output, and having an input coupled to the output of the logarithmic amplifier 458. The circuitry 452 further includes an amplifier 462 having an output, and an input coupled to the output of the logarithmic amplifier 458. The amplifier 462 has a gain which is set or selected so as to multiply the output of the logarithmic amplifier 458 (i.e., log(H)) by a constant bτ. Similarly, the circuitry 452 further includes an amplifier 464 having an output, and having an input coupled to the output of the multiplier 460. The amplifier 464 has a gain set so as to multiply the output of the multiplier 460 by a constant aτ. The circuitry 452 further includes a summing amp 466 having an output defining the node 454 at which the signal τ is produced. The summing amplifier 466 has an input coupled to the output of the amplifier 462, an input coupled to the output of the amplifier 464, and an input coupled to a signal representing the constant cτ.

The difference between the temperature signal T, and the temperature signal that correlates to a dew point of −20° C., τ, is provided at a second node 456. This difference is calculated as follows: θ=T−τ where θ is the differential temperature in Kelvins (clipped, in the illustrated embodiment, e.g. to 1.91≦θ≦86.09), and T is the temperature, in Kelvins beyond a −20° C. baseline, of the relative humidity sensor 455 (20≦T≦96).

More particularly, the circuitry 452 includes a differential amplifier 468 which has an output, an input coupled to the output of the summing amplifier 466, and further has an input coupled to a temperature sensor 470.

A signal representative of how far to go around the curve in FIG. 17 is provided at a node 472. The output at node 472 is a scaling factor (η) which determines the dew point vs. temperature slope; i.e., the rate at which the dew point changes as temperature changes. This scaling factor is calculated as follows:

η=aηlog(H) [symbol] 2=bηlog(H)+cη

where η is the scaling factor (0.4891≦η≦1), aη=0.03894, bη=0.17781, and cη=0.48911.

More particularly, the circuitry 452 includes an amplifier 474 having an output, and having an input coupled to the multiplier 460. The amplifier 474 has a gain which is set or selected so as to multiply the output of the multiplier 460 by a constant aη. Similarly, the circuitry 452 further includes an amplifier 476 having an output, and having an input coupled to the output of the logarithmic amplifier 458. The amplifier 476 has a gain set so as to multiply the output of the logarithmic amplifier 458 (i.e., log(H)) by a constant bη. The circuitry 452 further includes a summing amplifier 478 having an output defining the node 472 at which the signal η is produced. The summing amplifier 472 has an input coupled to the output of the amplifier 476, an input coupled to the output of the amplifier 474, and an input coupled to a signal representing the constant cη.

Both the output signals η and η can be defined by straightforward linear, second-order polynomial transformations of the log of relative humidity H.

The differential temperature (θ), scaled by the factor of η, and (logically) offset is provided at a node 480. This is calculated as follows:

t′=(θη)−20

where t′ is the dew point in ° C. (clipped, in the illustrated embodiment, to −18.5≦t′<55).

More particularly, the circuitry 452 includes a multiplier 482 having an output defining the node 480 at which the signal t′ is produced, having an input coupled to the output of the summing amplifier 478, and having an input coupled to the differential amplifier 468.

The above formulae used to design the circuitry 452 can be consolidated into a single formula as follows:

t′=(T−aτlog(H) [symbol] 2+bτlog(H)+cτ)(aηlog(H) [symbol] 2+bηlog(H)+cη)−20

Circuitry similar to that used in FIG. 16, like reference numerals indicating like components, takes the dew point signal t′ and determines a compensation factor for the MOS gas sensor 440.

In the illustrated embodiment, the output of the amplifier 442 defines a node 484 that generates compensation factors K ranging between 0.6619 (−18.5° C. dew point) and 1.463 (55° C. dew point). This range of compensation factors will compensate for environmental Rs/Ro values ranging from 1.511 (the reciprocal of 0.6619) through 0.6835 which is the lowest expected value in the fuel cell of the illustrated embodiment. A dew point value of 12.52° C. is equivalent to a compensation factor of unity.

The output of the multiplier 444 defines a node 486 which can be analogized to accepting the K signal and multiplying it by the uncorrected sensor resistance signal (w). If, for example, K=1.405 then a w of 1000 Ω would become a corrected gas concentration signal (w′) of 1405 Ω. This corrected resistance signal could then be imagined as being passed on to a final node where it is converted from a logarithmic resistance signal into a linear voltage signal. Because sensors of the type used for the sensor 440 vary in sensitivity to hydrogen, K is actually best handled as a log offset in a log amplifier in order that the effects of humidity can be variably “gained” while each circuit is calibrated for its sensitivity to hydrogen.

The circuitry 452 of FIG. 18 produces errors of less than 0.72° C. between 10 and 70° C. and relative humidities of between 5 and 100%.

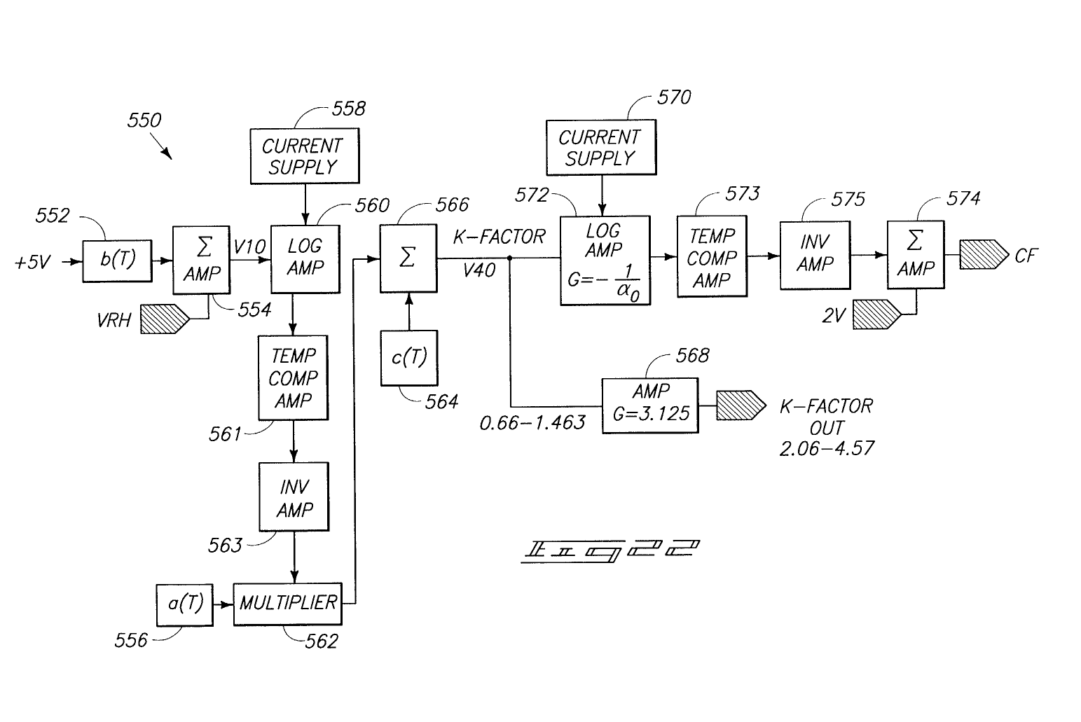

Between 20 and 60° C., the circuitry 452, which is provided by way of example, produces dew points with an average accuracy of ±0.15° C. and a median accuracy of 0.09° C. As illustrated in FIG. 19, the maximum errors occur along a curve where temperatures and relative humidities equal dew points of exactly 0° C. The reason for this is complex. These formulae produce their worse-case error at a temperature of 20.2° C. and a relative humidity of 25.7928% which is one of the points equal to a dew point of exactly 0° C. At this point, these formulae return a value—and error—of +0.7138° C. which is equal to a relative humidity error of +1.424%. Because even high quality relative humidity sensors have part-to-part interchangeability guarantees of only ±3%, the circuitry 452 (and formulae used to design it) produce dew points with accuracies exceeding that of its input sensors.

The source of the errors is caused by the way water's equation of state changes slightly below 0° C. On the 100% relative humidity line, this has the effect of making the slope change direction slightly below a temperature of 0° C. These errors can occur even at high temperatures as long as the dew points are equal to 0° C.

The point where the worse possible error occurs is at 20.2° C. and a relative humidity of 25.7928%. The fully saturated vapor pressure over water at 20.2° C. is 17.753 mmHg. By taking 25.7928% of this value, a vapor pressure of 4.579 mmHg is calculated. To determine what dew point is associated with a vapor pressure of 4.579 mmHg, you must determine what temperature water must be to achieve a fully saturated vapor pressure of 4.579 mmHg. The answer is 0° C. This is also the point where water's equation of state (its slope) makes a slight direction change. Since this formula provides a straight line between −20 and +70° C., the line will reach its maximum divergence from nature at 0° C. because this is where nature has a crook in the line. The errors tend to occur at dew points of 0° C.—not at ambient temperature of 0° C.—because any time a question of dew point is asked where the answer is 0° C., a comparison is necessarily made to water and its vapor pressure at an ambient temperature of 0 ° C.

Therefore, in one embodiment (not shown), the majority of this residual error is eliminated by moving the baseline dew point from −20° C. to 0° C. and then using a bipolar circuit for calculating η differently above and below 0° C. However, there is another source of error. The 2nd-order polynomial fit for ι (the shape of the curve at the base intersection) transitions through a temperature of 0° C. and the same effect occurs in this alternative embodiment too. With a −20° C. baseline, τ benefits from an improved fit by providing it too, in yet another alternative embodiment (not shown) with a bipolar amplifier with slightly different curves on both sides of the 16.896% relative humidity point (which is the 0° C. temperature point). However, using a bipolar amplifier for τ becomes a moot issue, in one embodiment, by moving the r baseline intersection upwards to 0° C., because the τ curve would then begin at 0° C. (at 100% relative humidity).

FIG. 20 is a flowchart illustrating logic used by digital circuitry or by a programmed general purpose computer, a microprocessor, or an integrated circuit, for determining dew point from relative humidity and temperature. In one embodiment, the digital circuitry that implements the logic of the flowchart is defined by the controller 250.

In step S1, relative humidity is input (e.g., from a relative humidity sensor such as the sensor 455). After performing step S1, the controller proceeds to step S2.

In step S2, temperature is input (e.g., from a temperature sensor such as the sensor 470). It will be readily apparent that steps S1 and S2 can be reversed. After performing step S2, the controller proceeds to step S3.

In step S3, dew point is calculated using a formula, such as:

t′=(T−aτlog(H) [symbol] 2+bτlog(H)+cτ)(aηlog(H) [symbol] 2+log(H)+cη)−20

where t′ is dew point; T is the temperature input in step S2; H is the relative humidity signal in full percentage counts; such as “50” for 50% relative humidity (1≦H≦100); aτ is a constant, e.g. 5.65; b is a constant, e.g. −44.3; cτ=66.14; and τ is temperature, in Kelvins beyond a −20° C. baseline, at which H correlates to a dew point of −20° C. (0.14≦τ≦66.14). After performing step S3, the controller proceeds to step S4.

In step S4, the dew point is output or used; e.g., for compensating a gas sensor for the effects of temperature and humidity on the sensor.

Using MOS gas sensors for sensing levels of gas concentrations is not a straightforward endeavor. This is due in part to large production variations in sensor response. For example, one particular model of MOS gas sensor, the Figaro TGS821, can vary (when measured at a specific temperature and relative humidity) from 1 kΩ to 10 kΩ at 100 PPM. The transfer function of a typical MOS gas sensor when plotted as a log-log plot of resistance vs. PPM is very close to a straight line. Another production variation that leads to design problems is that the slope of these lines on a log-log plot can vary substantially. For instance, the slope, or “alpha”, for this particular model of sensor can vary, from part to part, between −0.6 to −1.2. Consequently, a change in gas concentration from 50 PPM to 1360 PPM can produce anywhere from a 7:1 change in resistance to a 53:1 change due to this 2:1 range in alpha.

MOS sensors respond to the ratio of a target gas vs. oxygen via an oxidation/reduction reaction. Unfortunately, the presence of water vapor interferes with this simple relationship. The reasons for this interference are esoteric and will not be discussed in detail here. However, water vapor can be modeled as interfering with the relative availability of oxygen. The more water vapor present, the more MOS sensors behave as if there is less available oxygen.

Accordingly, no conclusion about target gas concentration can be drawn based on an MOS sensor's signal without accounting for the effects of water vapor. All sensors of this model will exhibit no change in their resistance reading when exposed to the following two gas mixtures:

| H₂ Concentration (PPM) | Dew Point (° C.) |
|---|---|
| 10 | 55 |
| 30 | −18.7 |

Additionally, all sensors of this model will exhibit no change in their resistance reading when exposed to these two gas mixtures:

| H₂ Concentration (PPM) | Dew Point (° C.) |
|---|---|
| 100 | 55 |
| 300 | −18.7 |

As can be seen from the above data, as compared to the −18.7° C. situation, exposing the sensor to a dew point of 55° C. produces the same effect as making two-thirds of the oxygen unavailable for the oxidation/reduction reaction. The sensor would accordingly interpret the hydrogen concentration (the mixing ratio) as having tripled unless it is exposed to only one-third as much hydrogen as is required at −18.7° C. This holds true for all sensors of this model—regardless of their alpha (gain)—because the sensor responds only to the mixing ratio of oxygen, hydrogen, and water vapor. Clearly, high-alpha MOS sensors naturally produce greater changes in resistance in response to changes in dew point and hydrogen concentration than do low-alpha sensors. It's noteworthy that the signal resistance of a TGS821 sensor with a mean alpha of −0.725 changes by a factor of 2.22:1 when the dew point changes from −18.7° C. to 55° C. It's even more noteworthy that this same change in dew point can produce resistance changes of as little as 1.93:1 (for α=−0.6) to as much as 3.74:1 (for a α=−1.2).

Referring to FIG. 11, which shows the normalized response of the TGS821, the width of line segments 502 and 504 denotes the effect of changing dew point from −18.5 to 55° C. The widths of these line segments, in response to variations in dew point, are constant for all TGS821 sensors regardless of their alpha. The illustrated alpha in FIG. 11 is −0.725 and this determines the height of line segment 500. If the graph were drawn with a steeper slope (alpha) of −1.2, the height of line segment 500 must necessarily increase; that is, the required change in resistance in response to changes in dew point would increase.

The typical method for compensating for MOS sensors' response due to environmental dependencies is to use microprocessor-based circuitry and look-up tables. Unfortunately, these tables are typically based upon an specific alpha (usually one that is close to the production mean). As described above, whenever sensors with alphas different from this mean are used, errors will result.

Therefore, to compensate MOS gas sensors for environmental dependencies (dew point), the raw transfer function of the sensor is taken (with its uncontrolled offset and gain) and an intermediate proxy version fitting a specified transfer function is created. In one particular embodiment, a constant-current source is made adjustable from 3 to 0.3 mA in order to compensate for the sensor's part-to-part variation in offset of 1 kΩ to 10 kΩ.

This current is then adjusted so the voltage drop across the sensor (Vsense) is 3.00 V@K=1. Vsense is then passed through signal conditioning circuitry where the specified transfer function is converted to 2.00 V@100 PPM@K=1 and its gain was then adjusted to equal 3.00 V@1000 PPM@K=1. As compared to Vsense, this effectively reverses the proxy signal's slope and normalizes its alpha to +1. With this method of fitting the sensor's raw transfer function into a specific logarithmic transfer function to create a first signal, a single function of dew point is then created and this second signal is summed into the first signal in order to accurately compensate the sensors for their environmental dependencies.

This method of offsetting the log-log plot of the sensor's response can be also accomplished digitally with a microprocessor. No lookup tables are required. One particularly practical implementation using a microprocessor compensates for the sensor's part-to-part offset variation by calibrating a constant current source as described above in an all-analog method. Thus, the dynamic range of the analog circuitry is narrowed on the order of a factor of ten. The sensor's alpha would therefore be the only digital calibration variable that would need to be stored.

In order to compensate for dew point, a method of determining dew point is required. Instead of using expensive chilled mirror equipment, it would be desirable to calculate dew point, given inputs of temperature and humidity, in a simpler manner.

The dew point temperature, or DPT, is that temperature where the air is completely saturated with moisture. If the temperature were to be decreased below the dew point temperature, precipitation would occur.

Dew point (DPT) can be looked up on any readily available Psychometric chart. Observation of the Psychometric chart reveals that Relative Humidity appears as a family of curves.

Re-plotting the Psychometric chart with Dew point temperature on the z axis, Temperature on the x axis, and Relative Humidity on the y axis, yields a plot that takes on the shape of a sail. Re-scaling the Dew point temperature on the vertical axis between the two end-point values allows the creation of a new chart that has a correction factor, K, as the vertical axis.

Observation of this new chart quickly reveals the logarithmic nature of the Humidity curves. Taking “slices” of this sail at constant relative humidity values and subsequently at constant temperature values reveals that for a fixed value of relative humidity, a linear family of curves is produced as a function of temperature, T.

Circuit implementation requires an equation of the transfer function. One approach is to perform a polynomial least squares curve fit. Trying to curve fit with a second order equation produces errors of about 7-10%, while using a third order equation limits the error to within about 1%, which is sufficient accuracy for some instrumentation applications. Circuit implementations of third and higher order equations are difficult and expensive to reliably design and produce. An alternative approach is to try a curve fit of the log function of relative humidity using a least squares approach, which is described below as one alternative embodiment of the invention. The value of dew point temperature has been linearly scaled to fit the MOS gas sensor correction factor for dew point.

One very specific circuit implementation for implementing this dew point calculation methodology, and dew point compensation methodology will now be described in order to very easily enable one of ordinary skill in the art to practice the invention; however, other alternative circuit constructions and component values can readily be employed.

FIGS. 21A and B, when assembled together, show a block diagram of circuitry 500 for providing a linear output signal from a hydrogen sensor using more simplified circuitry. The implementation described below produces a linear output signal and can be used with sensors having different alpha values (see description of a above, described in connection with FIG. 16). Alpha (α) is a sensor specific property, indicative of the sensitivity of a specific sensor.

The circuitry 500 described below compensates a hydrogen sensor for dew point instead of compensating separately for relative humidity and temperature, and an analog circuit is described that does not require complex look-up tables.

The response of a hydrogen sensor to H₂ levels is as shown in FIG. 11. The x-axis is hydrogen concentration in PPM and the y-axis is Rs/Ro or normalized resistance of the sensor (Rs is the resistance of the sensor and Rs/Ro is normalized resistance of the sensor such that 100 PPM hydrogen concentration has an Rs/Ro of 1. This response is a straight line on a graph having a logarithmic x-axis and a logarithmic y-axis.

A straight line can be represented by a function: Y=MX+B

$$or \frac{Rs}{Ro} = 10 ⋀ ( αlog ( PPM ) - 2 α ) \qquad ( 1 )$$

where the symbol “ [symbol] ” represents an exponential.

As described above, the inventors have determined that the influences of temperature and humidity have the effect of shifting this line up or down. Mathematically this means that there is an offset β such that

$$\frac{Rs}{Ro} = 10 ⋀ ( αlog ( PPM ) - 2 α + β ) \qquad ( 2 )$$

It may be desirable to produce a linear output, regardless of the effects of temperature and relative humidity. For example, assume that it is desired that if the concentration of hydrogen is 100 parts per million, the output of the circuitry coupled to the hydrogen sensor is 3.75 volts and that if the concentration of hydrogen is 1000 parts per million, the output of the circuitry coupled to the hydrogen sensor is 1.50 volts. This can be described by a line:

Vo=4−0.0025×PPM (3)

where PPM represents sensed gas concentration in PPM.

A problem is how to get from equation 2 to equation 3. An additional problem is how to account for huge variations in resistance at a fixed PPM level of 100 PPM. In circuitry 500 shown in FIGS. 21A-B, a voltage is obtained from gas sensor resistance by driving the gas sensor 503 with a current source 502. In the illustrated embodiment, the sensor is a hydrogen sensor; however, in alternative embodiments, sensors for sensing different types of gasses can be used with circuitry similar to the circuitry shown. The current source 502 is altered to calibrate out the wide variation in resistance, Ro. In the embodiment shown in FIGS. 21A-B, three volts was selected as a calibration voltage level at 100 PPM (referred to as the Ro resistance) and current is varied such that an output of three volts is obtained from the sensor at 100 parts per million concentration. The level of current that provides a Vs=3 v at 100 PPM is defined as Io. Once set, Io is fixed. Therefore, Vs=IoRs. Vs is shown in FIGS. 21A-B. If Rs is described by equation 2, then

Vs=IoRo10 [symbol] (αlogPPM−2α+β)

Here, β corrects Vs for the effects due to temperature and relative humidity.

but IoRo=3v, therefore,

Vs=3×10 [symbol] (αlogPPM−2α+β) (4)

To remove the exponential part of equation 4, the log of Vₛ is taken (by logarithmic amplifier 510) and is called V₁.

V1=logVs=log3+(αlogPPM−2α+β) (5)

To remove the log3 term, it is subtracted out (by summing amplifier 516):

V2=V1−log3

V2=αlogPPM−2α+β (6)

Alpha (α) is then factored out (by divider 518, inverting amplifier 520 and inverting amplifier 522):

$$V3 = \frac{V2}{a} = Log PPM - 2 + ( \frac{β}{α} ) \qquad ( 7 )$$

Define Correction Factor, CF as:

$$CF = ( 2 - \frac{β}{α} )$$

Add CF using summing amplifier 524 to get

$${V4}^{′} = V3 + 2 - ( \frac{β}{α} )$$

A remaining problem is relating β to the dew point sail.

The inventors found that

$$β = α ( \frac{1}{{α}_{0}} ) log ( \frac{1}{K} )$$

where K is dew point temperature scaled using specific aₖ and bₖ factors and where α₀ is the normalizing alpha for which the aₖ and bₖ factors were optimized. For the example of Figaro's model TGS821 sensor, the aₖ factor of 0.0109 and a bₖ factor of 0.86352 were both developed referencing an α₀ of −0.725. Different MOS sensors require different aₖ and bₖ factors to describe the extent of their environmental dependencies. After compensating for dew point, the equation for the compensated Rs becomes

$$Rs = Ro \times 10 ⋀ [ α ( log PPM + ( \frac{1}{{α}_{0}} ) log ( \frac{1}{K} ) - 2 ) ] \qquad ( 9 )$$

Vo=Io×Rs=3 (10)

$$\begin{matrix}Vs = 3 \times 10 ⋀ [ α ( log PPM + ( \frac{1}{{α}_{0}} ) log ( \frac{1}{K} ) - 2 ) ] & ( 11 ) \\ V1 = log Vs = log 3 + α ( log PPM + ( \frac{1}{{α}_{0}} ) log ( \frac{1}{K} ) - 2 ) & ( 12 )\end{matrix}$$

V2=V1−log3 (produced by summing amplifier 516) (13)

$$V2 = α ( log PPM + ( \frac{1}{{α}_{0}} ) log ( \frac{1}{K} ) - 2 ) \\ V3 = \frac{V2}{α} = log PPM + ( \frac{1}{{α}_{0}} ) Log ( \frac{1}{K} ) - 2 \qquad ( 14 )$$

At this stage, a correction factor, CF, needs to be added by summing amplifier 524 to be able to get to

V4=logPMM

In order to do this,

$$CF + ( \frac{1}{{α}_{0}} ) log ( \frac{1}{K} ) - 2 = 0 \\ CF = 2 - ( \frac{1}{{α}_{0}} ) log ( \frac{1}{K} ) \\ Since log ( \frac{1}{K} ) = {log ( K )}^{- 1} = - log ( K ) \qquad ( 15 )$$

this reduces to

$$CF = 2 + ( 1 / {α}_{0} ) ( K ) \qquad ( 16 )$$

∴Let V4=V3+CF (17)

V4=logPMM

V5=Gs×10 [symbol] V4 Gs=−0.0025 (18)

V5=−0.0025 PPM

V6=4+V5=Vout (19)

Vout=4−0.0025 PPM (20)

Although this appears to be what is required, a problem with implementing an actual circuit is that V4>0 and the antilog amp calculates 10^{-V4}, so the Vout had a positive slope whereas a negative slope is desired. To solve this problem:

let V4=logPMM as above

but let V5=4−logPMM (produced by amplifier 526) (21)

let V6=10×10 [symbol] (−V5) (produced by anti-log amplifier 530)

V6=10×10 [symbol] (−4+logPPM)

V6=10×10 [symbol] (logPMM−4) (22)

V6=10×10 [symbol] logPMM×10⁴

Therefore, V6=10⁻³×PPM (23)

let V7=2.5(V6) (produced by amplifier 525 with gain of 2.5) (24)

V7=0.0025 PPM

Vout=4−V7 (produced by summing amp 534) (25)

Vout=4-0.0025PPM (26)

This is what is needed. This analysis was used to design the circuitry 500 shown in FIGS. 21A-B. The circuitry shown in FIGS. 21A-B is one very specific embodiment and should not be used to interpret the scope of the claims below. For every model number or value given, other models or values can, of course, be employed. Further, other circuit designs could be employed for each functional block.

The circuitry 500 includes a current supply 502 adapted to drive a hydrogen sensor 503 as described above. The circuitry 500 further includes an alarm circuit 504 coupled to the current supply 502, and a buffer 506 having an input coupled to the lo supply 502. The buffer 506 has an output on which a signal Vs is produced.

The circuitry 500 further includes a current supply 508, and a logarithmic amplifier 510 coupled to the current supply 508 to receive current from the current supply 508, having an input coupled to the output of the buffer 506, and having an output. The circuitry 500 further includes a temperature compensation amplifier 512 having an input coupled to the output of the logarithmic amplifier 510 and having an output. The circuitry 500 further includes an inverting amplifier 514 having an input coupled to the output of the temperature compensating amplifier 512 and having an output on which a signal V1 is produced. In one embodiment, a quad amplifier integrated circuit is used to build the current supply 508, the logarithmic amplifier 510, the temperature compensation amplifier 512, and the inverting amplifier 514; however, other constructions are, of course possible, such as those using separate or different integrated circuits in different parts of the circuitry 500 or by manufacturing a single integrated circuit to perform all the functions of the circuitry 500.

The circuitry 500 further includes summing amplifier 516 which has an input coupled to the output of the inverting amplifier 514, having an input coupled to a signal representing −log(3) and having an output on which a signal V2 is produced, where V2 is the sum of V1 and −log(3). The circuitry 500 further includes a divider 518 having an input coupled to the output of the summing amplifier 516, having an input coupled to a signal representing α (alpha), and having an output. In the illustrated embodiment, the divider is defined by an operational amplifier; however, in alternative embodiments, other circuit elements could be used.

The circuitry 500 further includes an inverting amplifier 520 having an input coupled to the output of the divider 518 and having an output. The circuitry 500 further includes an inverting amplifier 522 having an input coupled to the output of the inverting amplifier 520 and having an output on which a signal V3 is produced. The signal V3 represents V2/α. The circuitry 500 further includes a summing amplifier 524 that has an input coupled to the output of the inverting amplifier 520, an input coupled to a signal representing the correction factor (CF), and an output on which a signal V4 is produced. The signal V4 represents the sum of V3 and the correction factor CF.

The circuitry 500 further includes a summing amplifier 526 having an inverting input coupled to the output of the summing amplifier 524, having a non-inverting input coupled to a signal representing the value 4, and having an output on which a signal V5 is produced. The signal V5 represents the difference 4−V4. The circuitry 500 further includes a current supply 528, and an anti-log amplifier 530 receiving current from the current supply 528, having an input coupled to the output of the summing amplifier 526, and having an output on which a signal V6 is produced. In the illustrated embodiment, the current supply 528 and the anti-log amplifier 530 are defined by two operational amplifiers; however, in alternative embodiments, different circuit elements could be employed.

The circuitry 500 further includes an amplifier 532 having an input coupled to the output of the anti-log amplifier 530 and having an output on which a signal V7 is produced. The circuitry further includes a summing amplifier 534 having an input coupled to the output of the amplifier 532 and having another input coupled to a signal representing a value 4, and having an output on which the desired signal Vout, described above, is produced. (Vout=4−0.0025×hydrogen concentration in PPM).

FIG. 22 shows a block diagram of circuitry 550 for generating the correction factor CF used in the block diagram of FIGS. 21A-B and for generating the dew point compensation factor K described above in connection with FIGS. 16 and 18. The K factor, as described above, was generated using log functions and a polynomial. This is more cumbersome to implement in analog circuitry than the following approach, which is a simplification. The following observations were used in designing the circuitry: 1) for a fixed temperature, the dew point compensation factor K appears to vary in a logarithmic fashion versus percentage relative humidity; and 2) for a fixed percentage relative humidity, K appears to vary linearly versus temperature (T). After considering these observations, it was decided to model the K factor using the following function:

K(H,T)=(a₁T+a₀)log[(b₁T+b₀)+V_{H}]+c₁T+c₀

A least squares technique was used to find a₁, a₀, b₁, b₀, c₁, and c₀. Define a(T)=a₁T+a₀, b(T)=b₁T+b₀, and c(T)=c₁T+c₀.

The circuitry 550 of FIG. 22 provides K by taking b(t), adding it to V_{H}, taking the log of the sum, multiplying the sum by a(T), then adding c(T). The K factor, after multiplication by a scaling factor, e.g. 3.125, is available as an output for testing or other desired purposes; however, it is converted to the correction factor CF for purposes of the circuitry 500. This conversion is performed by using CF=2−(1/α₀)log(1/K).

The circuitry 550 includes circuitry 552 defining b(T). The circuitry 552 receives an input of 5 volts and has an output. The circuitry 550 further includes a summing amplifier 554 having an input coupled to the output of the circuitry 552, also having an input coupled to a voltage signal V_{H}, representing relative humidity, from a humidity sensor, and having an output.

The circuitry 550 further includes circuitry 556 defining a(T) and having an output. The circuitry 550 further includes a current supply 558, and a logarithmic amplifier 560 coupled to receive current from the current supply 558. The logarithmic amplifier 560 further has an input coupled to the summing amplifier 554, and has an output. The circuitry 550 further includes a temperature compensating amplifier having an output and having an input coupled to the output of the logarithmic amplifier 560; and an inverting amplifier 563 having an output, and having an input coupled to the output of the temperature compensating amplifier 561. The circuitry 550 further includes a multiplier 562 having an input coupled to the circuitry 556 defining a(T), having an input coupled to the output of the inverting amplifier 563, and having an output.

The circuitry 550 further includes circuitry 564 defining c(T), and a summing amplifier 566 coupled to add c(T) to the output of the multiplier 562 to produce the K factor, V40.

The circuitry 550 further includes an amplifier 568 having an input coupled to the K factor output of the summing amplifier 566, having a gain of 3.125, and having an output defining a signal representative of dew point.

The circuitry 550 further includes a current supply 570 and a logarithmic amplifier 572 having an input coupled to the K factor output of the summing amplifier 566, having a gain of (−1/α₀), and having an output. The circuitry further includes a summing amplifier 574 having an input coupled to a signal representing the value 2 (e.g., 2 volts), having an input coupled to the output of the logarithmic amplifier 572, and having an output defining the correction factor CF which is used by the amplifier 524 in the circuitry 500.

The remaining figures show minute details of the circuitry shown in FIGS. 21 and 22, for the sake of completeness only; other variations are of course possible, and these specific circuit elements and values should not be used to limit the claims. As specific circuit elements could easily be changed, only an overview will be provided of these circuits.

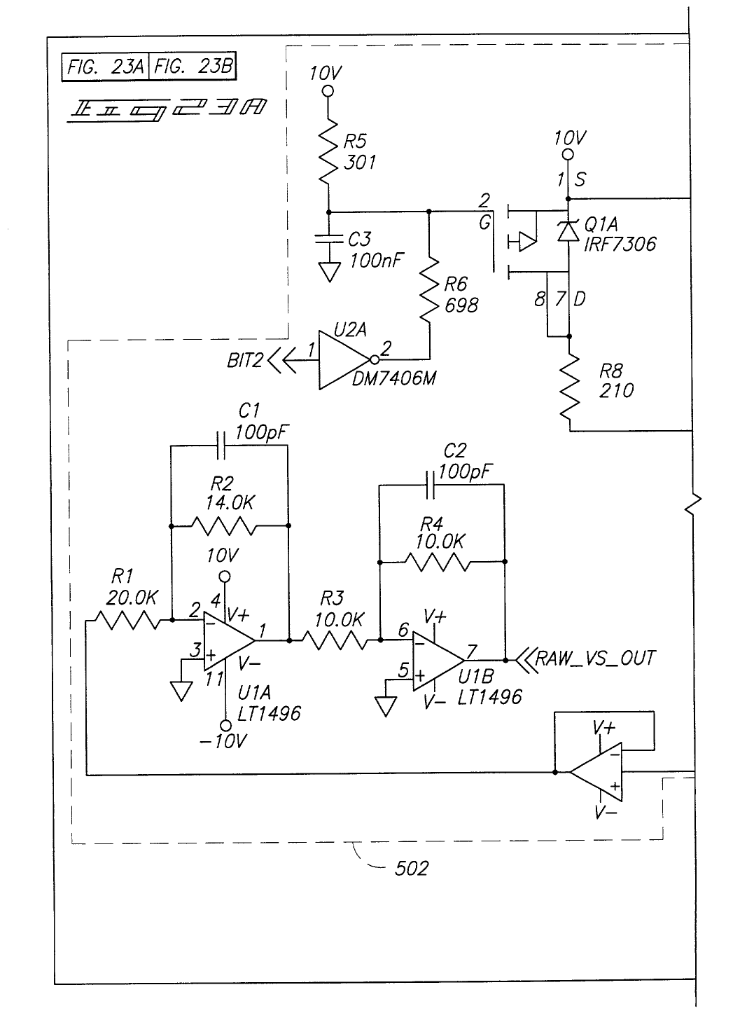

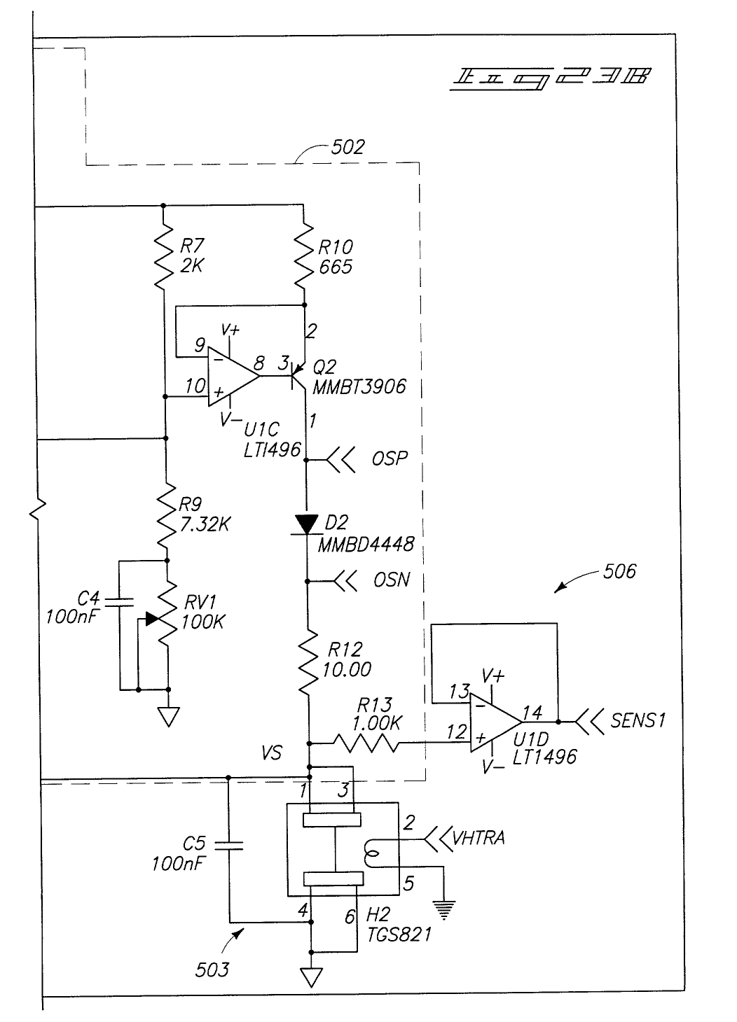

FIGS. 23A-B show construction details of circuitry that could be used to define one embodiment of the lo supply 502 to drive the sensor 503. The lo supply 502 includes an op amp U1A and associated resistors R1 and R2 and capacitor C1; an op amp U1B, and associated resistors R3, R4, and C2, having an inverting input coupled to the output of op-amp U1A. The inverting input of op-amp U1A is coupled to the hydrogen sensor 503 via a buffer amp and the resistor R1. The output of op-amp U1B defines a raw output voltage RAW_VS_OUT. U2A is an inverter and is coupled to a resistor R6 which in turn is coupled to a voltage defined between resistor R5 and capacitor C3 which are series coupled between 10 volts and ground. The voltage is applied to the gate of a transistor Q1A. Transistor Q1A includes a source coupled to 10 volts and a drain coupled to resistor R8. A variable resistor RV1 and associated capacitor C4 are coupled in series with resistor R9 and resistor R7 to define a voltage divider. The Io supply 502 further includes an op-amp U1C having a non-inverting input coupled between resistor R7 and resistor R9 which is also where the other end of resistor R8 is coupled. The op-amp U1C further includes an inverting input, and the Io supply 502 further includes a resistor R10 coupled between an inverting input of the op-amp U1C and 10 volts. A transistor Q2 has a gate coupled to the output of the op-amp U1C and a collector-emitter path coupled between resistor R10 and a diode D2. The lo supply 502 further includes a resistor R12 coupled between the diode D2 and the sensor 503.

FIGS. 23A-B further show an op amp U1D, and associated resistor R13, together defining one embodiment of the buffer 506.

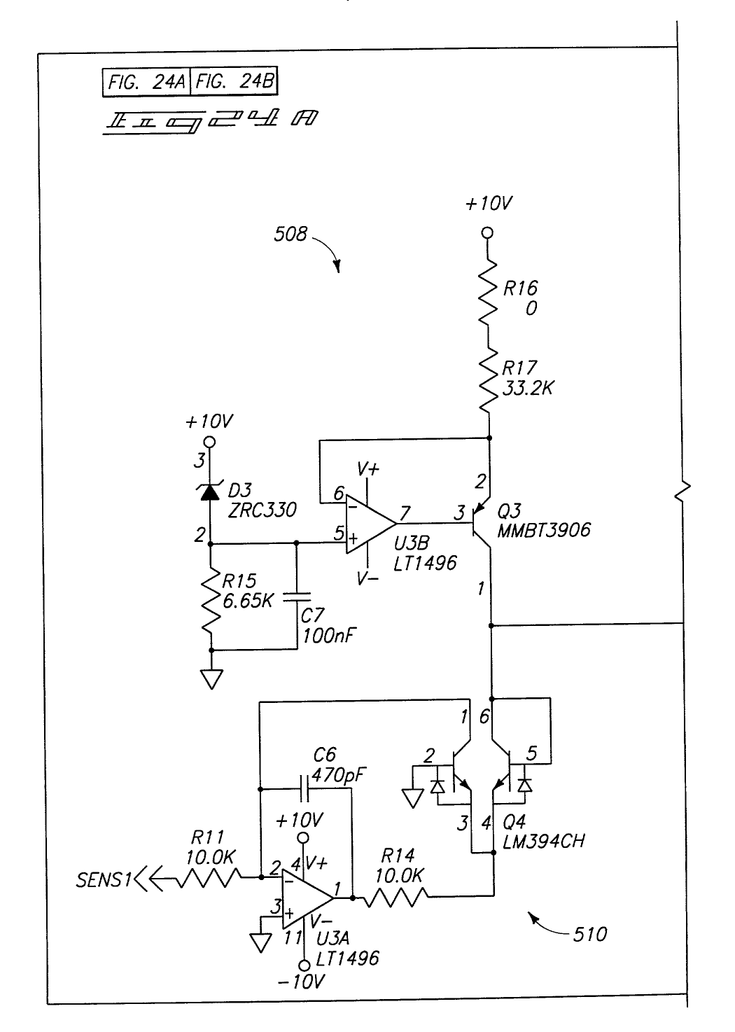

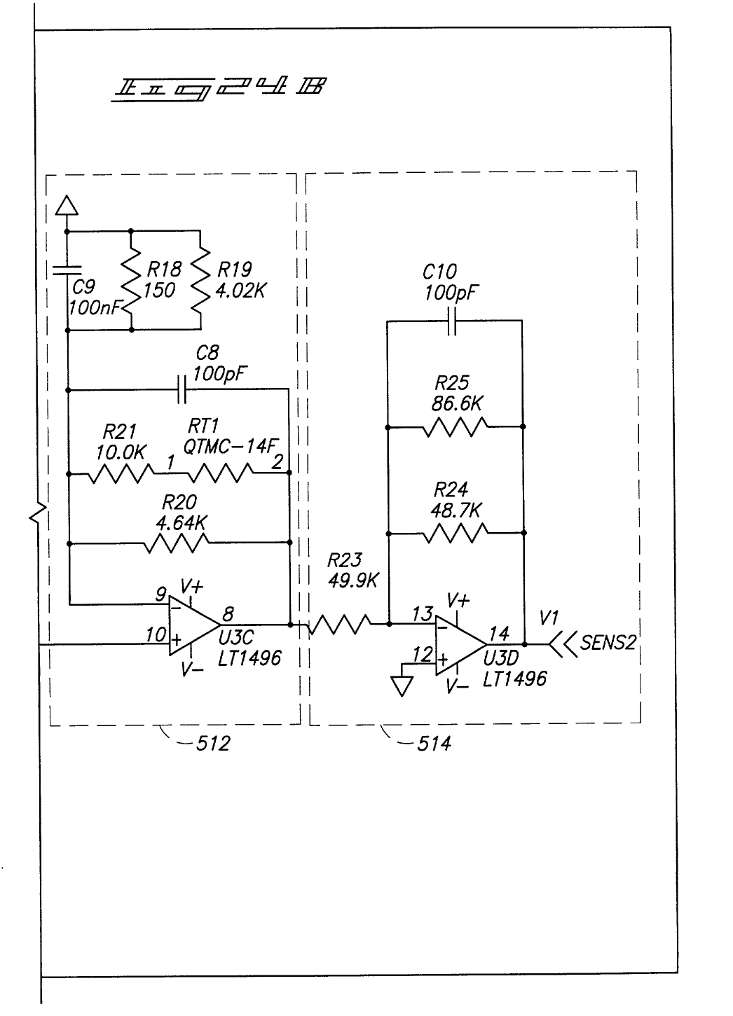

FIGS. 24A-B show circuitry defining the current source 508 and the logarithmic amplifier 510, in one embodiment of the invention. The circuitry 510 includes an op-amp U3A having an inverting input coupled to the output of the buffer 506 of FIGS. 23A-B, via resistor R11, having a non-inverting

input coupled to ground, having an associated capacitor C6 and having an output. The circuitry 510 further includes difference amplifier circuitry Q4 having one branch coupled between the output of the op-amp U3A via resistor R14 and the inverting input of the op-amp U3A. The circuitry 508 includes constant voltage circuitry defined by zener diode D3, resistor R15, and capacitor C7, op-amp U3B. and transistor Q3. Transistor Q3 has a collector-emitter path coupled between a voltage source, via resistors R16 and R17, and the second branch of the difference amplifier circuitry Q4.

FIGS. 24A-B also show construction details of one embodiment of a temperature compensating amplifier 512 including an op-amp U3C having a non-inverting input coupled to the current source, and having temperature sensitive element RT1 associated with the op-amp U3C, along with resistor R21, resistor R20, capacitor C8. Capacitor C9, resistor R18, and resistor R19 are coupled together in parallel between a voltage source and the inverting input of the op-amp U3C.

FIGS. 24A-B also show construction details of one embodiment of an inverting amplifier 514, including op-amp U3D having an inverting input coupled to the output of the temperature compensating amplifier 512, via a resistor R23, having a non-inverting input coupled to ground, and having associated resistors R24 and R25 and an associated capacitor C10. The output of the op-amp U3D defines voltage V1 of FIGS. 21A-B.

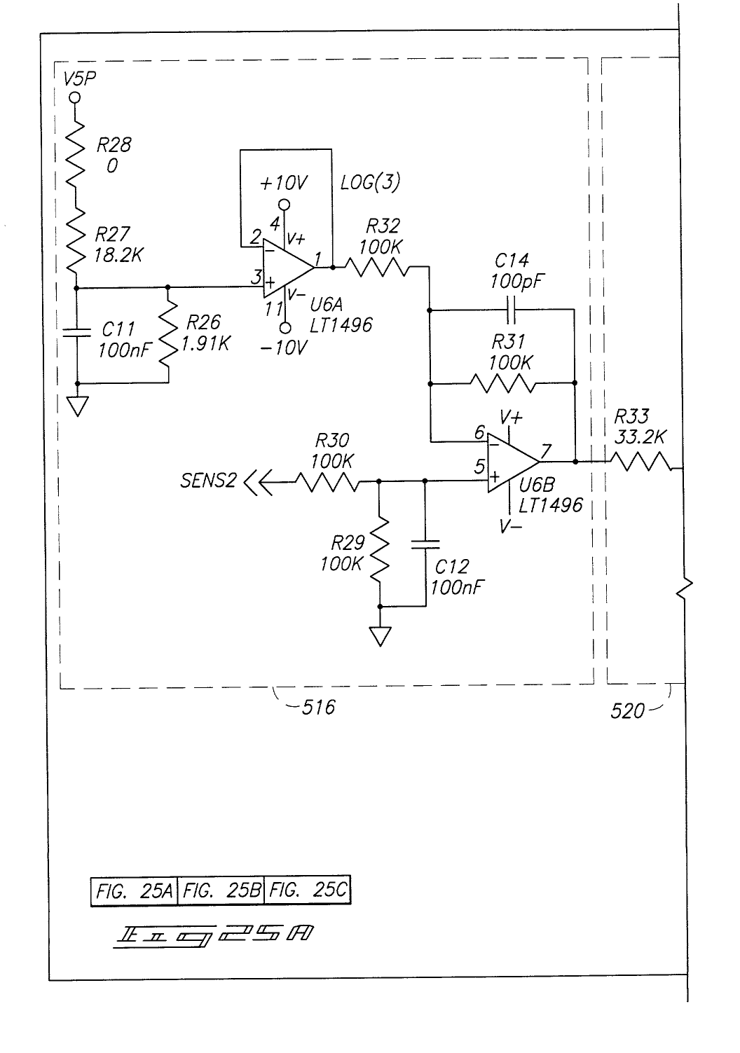

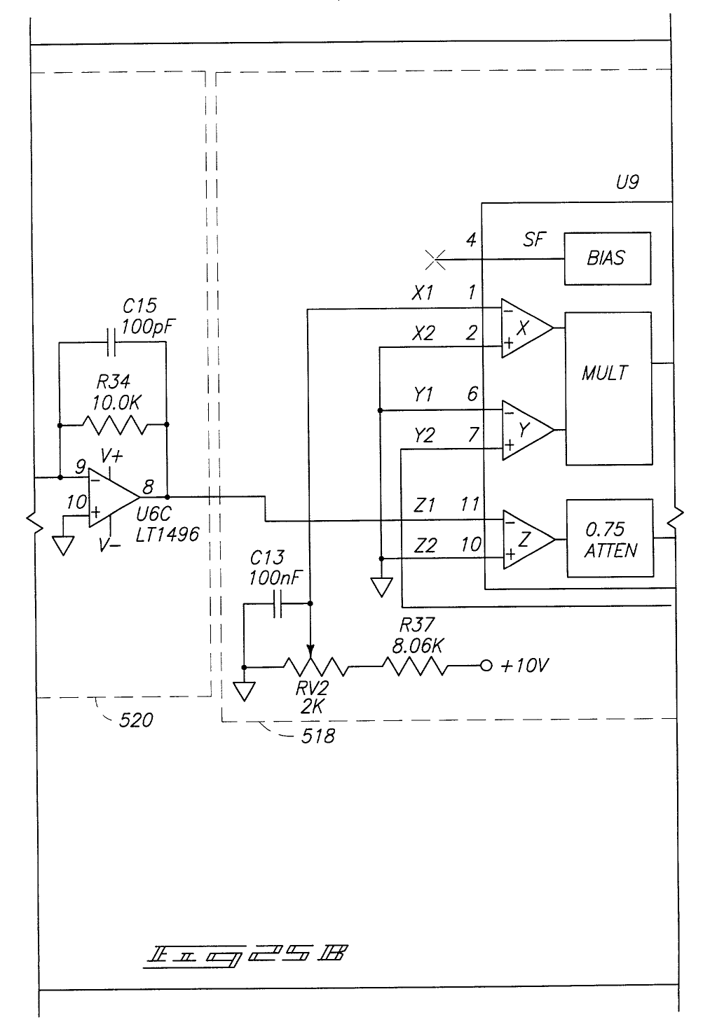

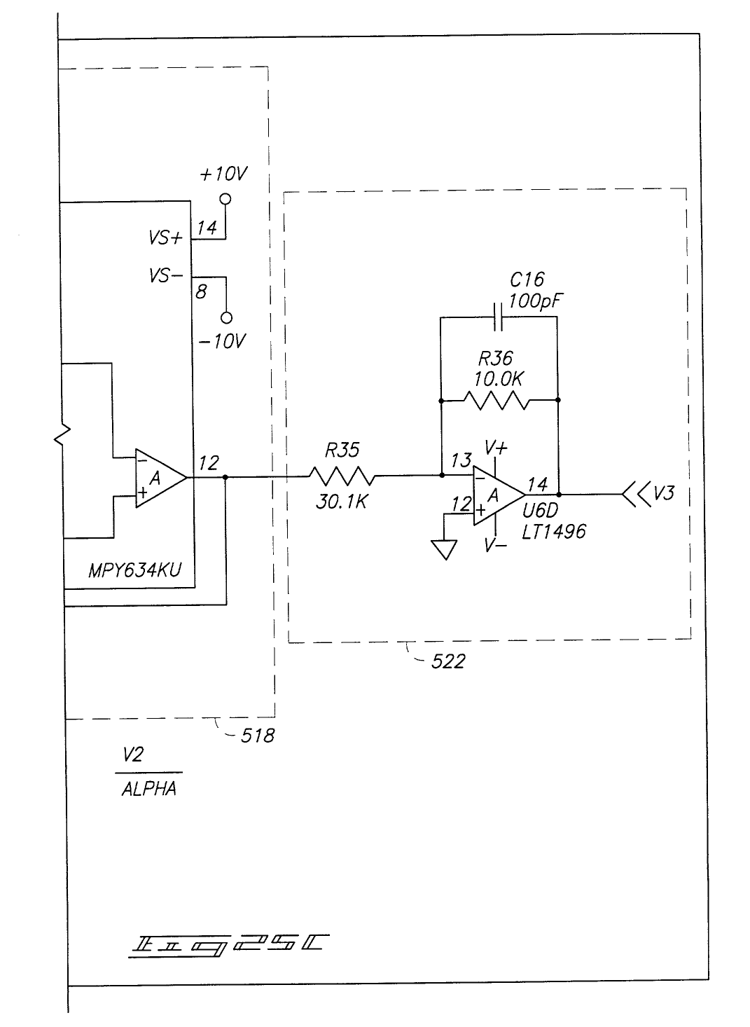

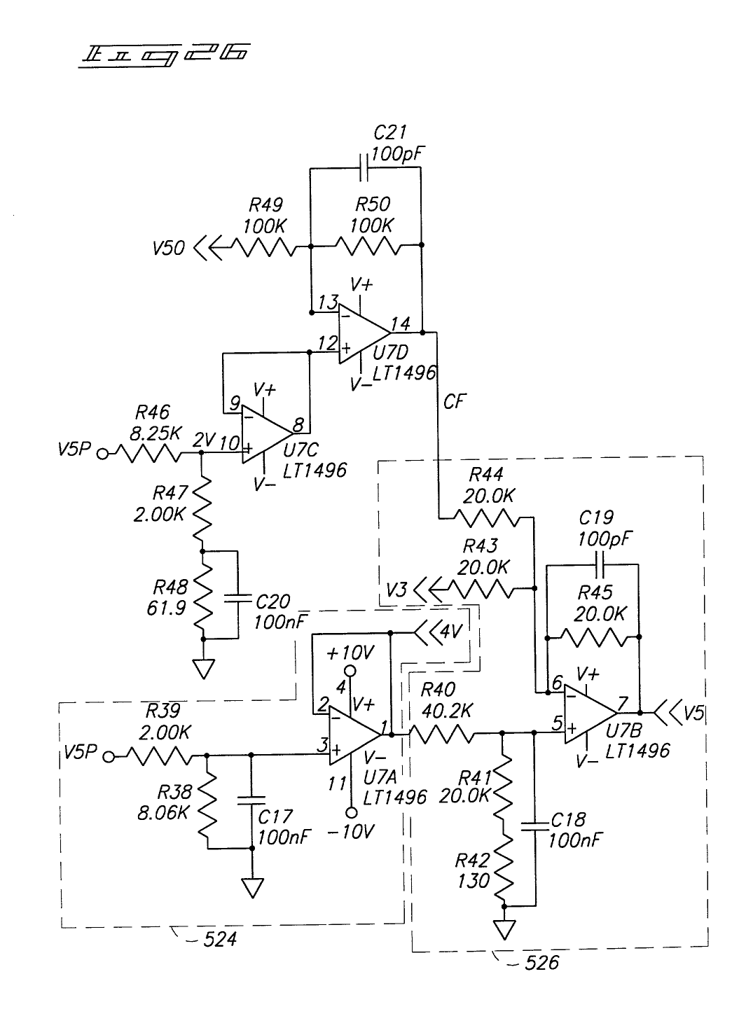

FIGS. 25A-C show construction details of one embodiment of summing amplifier 516, to add the output of the inverting amplifier 514 to −log(3). The circuitry shown in FIGS. 25A-C includes circuitry defining log(3), including op-amp U6A configured as a buffer and having a non-inverting input coupled to resistors R28, R27, R26 and capacitor C11. The circuitry 516 further includes op-amp U6B having an inverting input coupled to the output of the inverting amplifier 514 of FIGS. 24A-B. Resistor R29 and capacitor C12 are coupled in parallel between the non-inverting input of the op-amp U6B and ground. The circuitry 516 further includes a resistor R31 and capacitor C14 associated with the op-amp U6B, and log(3), the output of the op-amp U6A, is coupled to the inverting input of the op-amp U6B. The circuitry 516 further includes an op-amp U6C having a non-inverting input coupled to ground, and having an inverting input coupled to the output of op-amp U6B via resistor R33. Circuitry 516 further includes a resistor R34 and capacitor C15 associated with the op-amp U6C.

FIGS. 25A-C further show construction details of one embodiment of a divider 518. The divider 518 includes an integrated circuit, an analog multiplier model MPY634KU available from Burr-Brown, Tucson Ariz. (now Texas Instruments), and an associated variable resistor RV2, capacitor C13, and resistor R37.

FIGS. 25A-C further show construction details of the inverting amplifiers 520 and 522. In the illustrated embodiment, op-amps U6C and U6D with associated resistors R36, R33, R34, capacitor C16 and resistor R35 define the inverting amplifiers 520 and 522. Amplifier 522 is coupled to the output of the divider 518.

FIG. 26 shows construction details of circuitry defining the correction factor CF, circuitry defining 4 volts, and circuitry defining the summing amplifier 526, in accordance with one embodiment. More particularly, the correction factor CF is defined by circuitry including op-amp U7C, op-amp U7D, resistors R49 and R50 and capacitor C21 associated with op-amp U7D, and resistors R46, R47, and R48 defining a voltage of 2 volts and an associated capacitor C20 with op-amp U7C.

Circuitry 524 defines 4 volts and includes an op-amp U7A, resistors R39 and R38, and capacitor C17. Op-amp U7A has an output.

Circuitry 526 includes an op-amp U7B having an inverting input coupled to the voltage V3 of FIGS. 21A-B, via resistor R43, and also to the correction factor CF, via resistor R44. The op-amp U7B also has an associated resistor R45 and capacitor C19, and has a non-inverting input coupled to the output of the 4 volt generator, and resistors R40, R41, R42, and capacitor C18. The circuitry 526 together performs the function of 4−CF−V3.

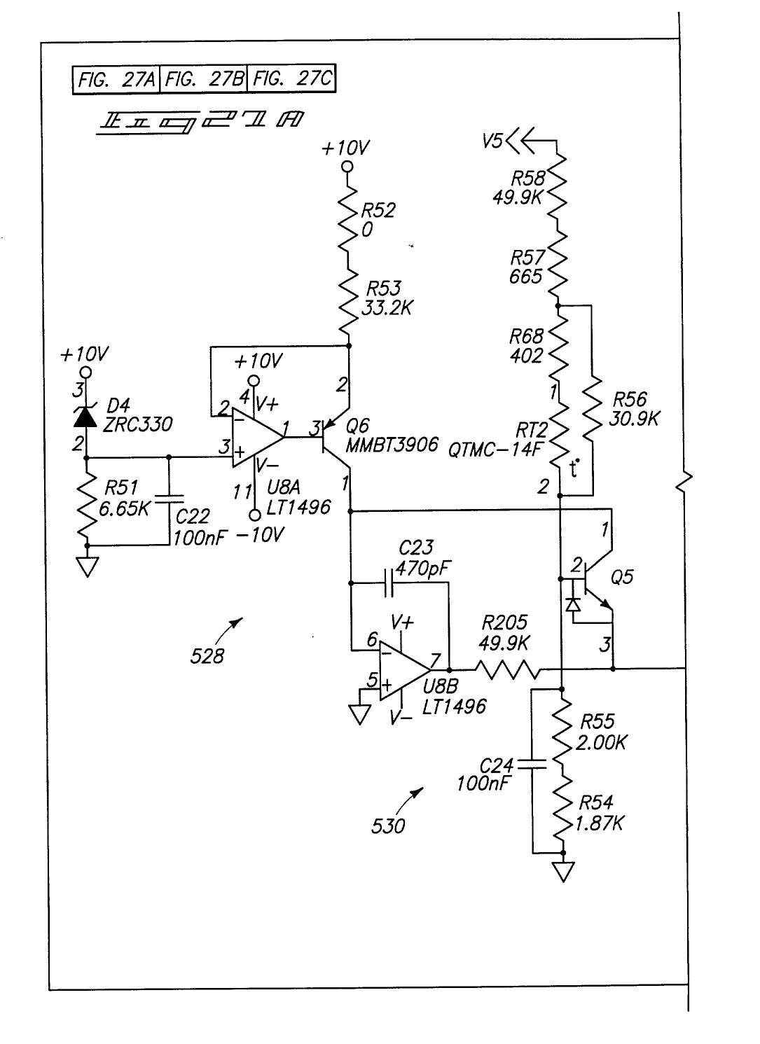

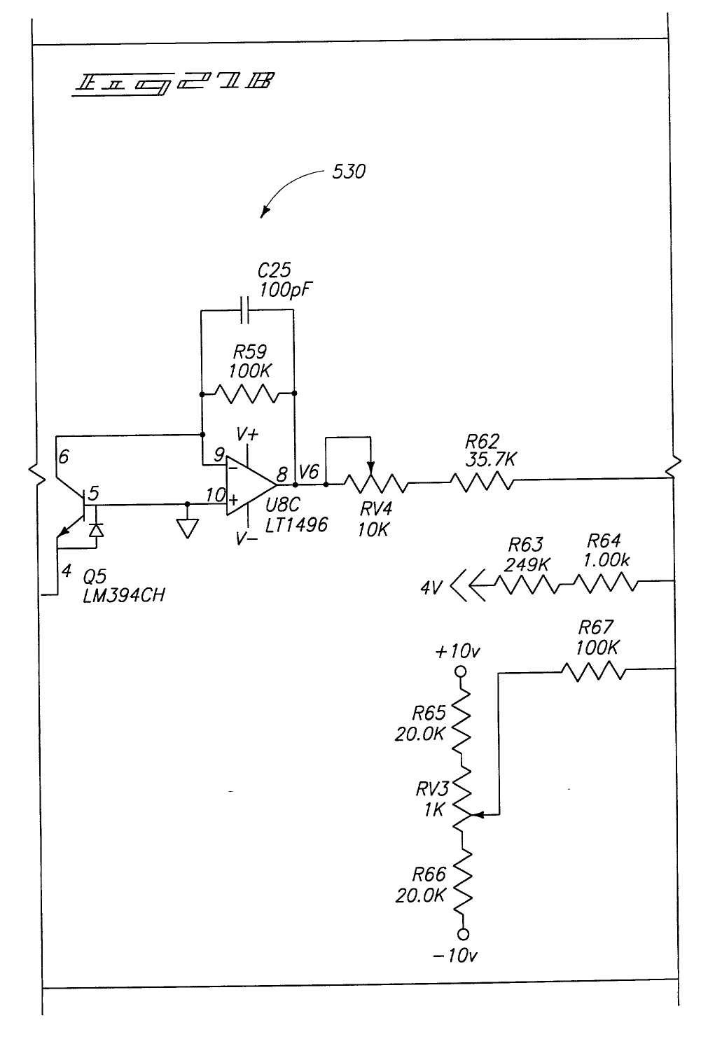

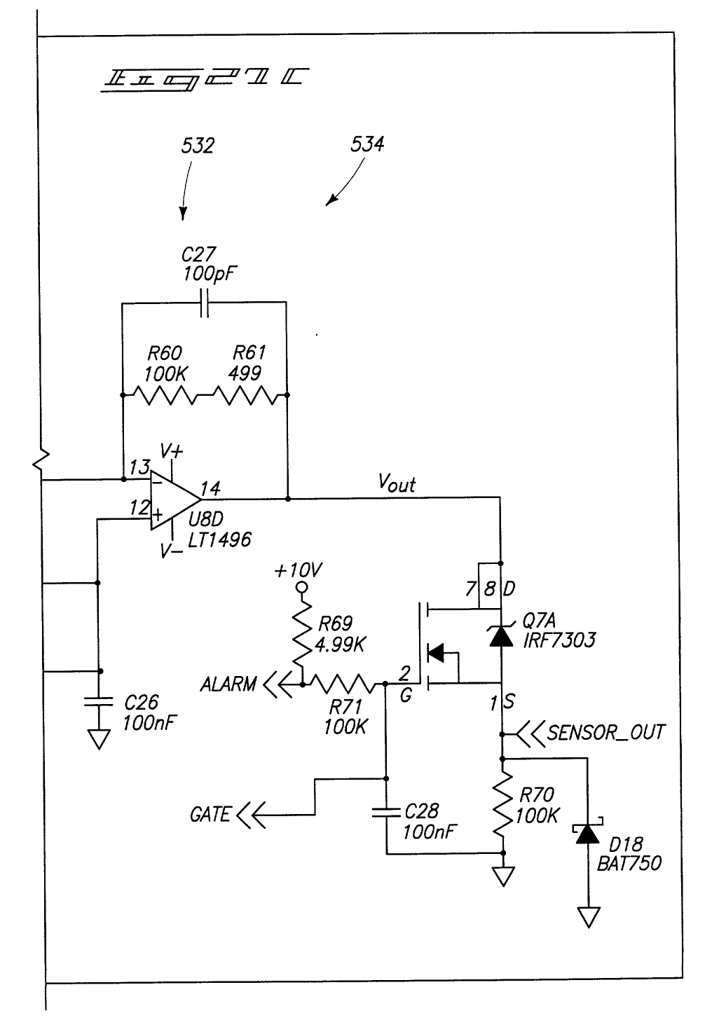

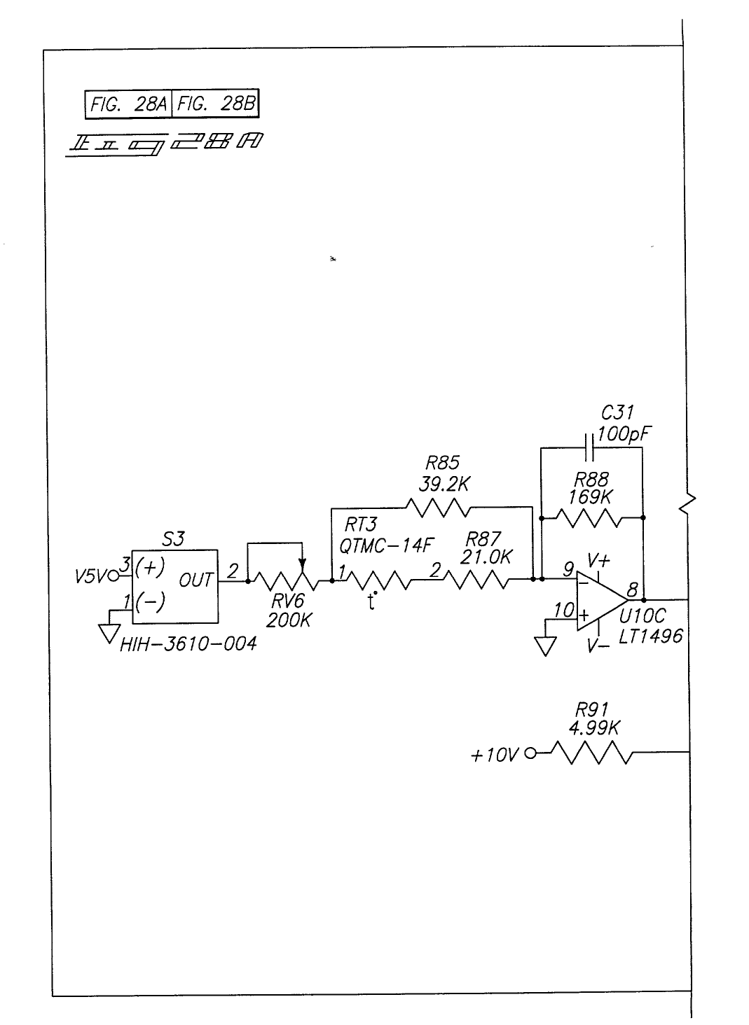

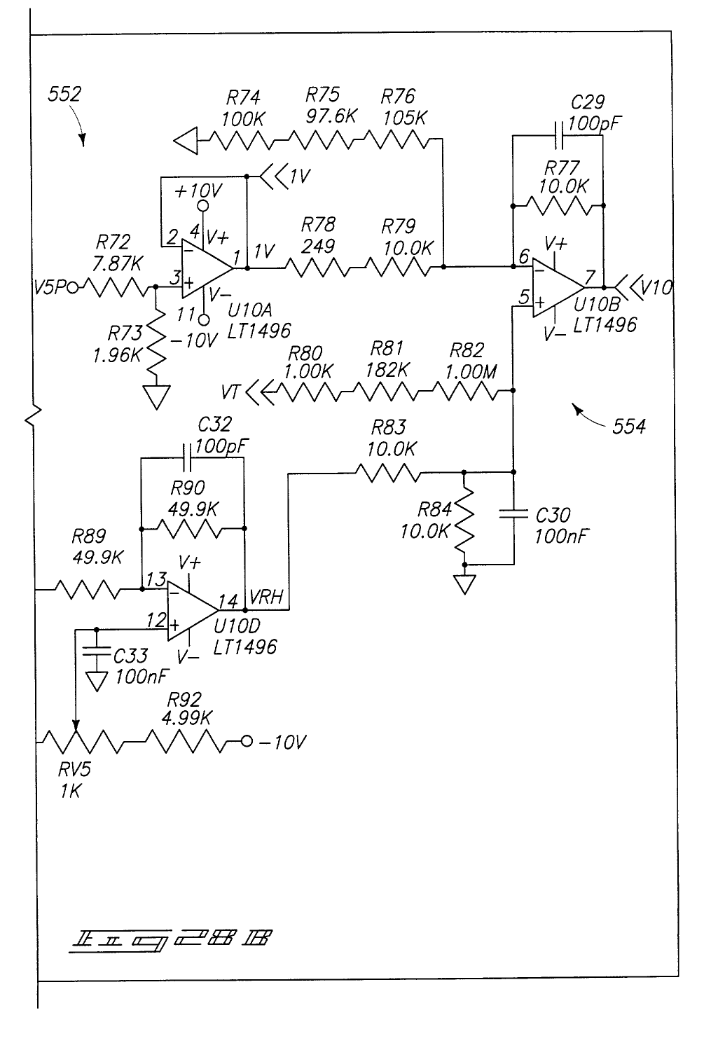

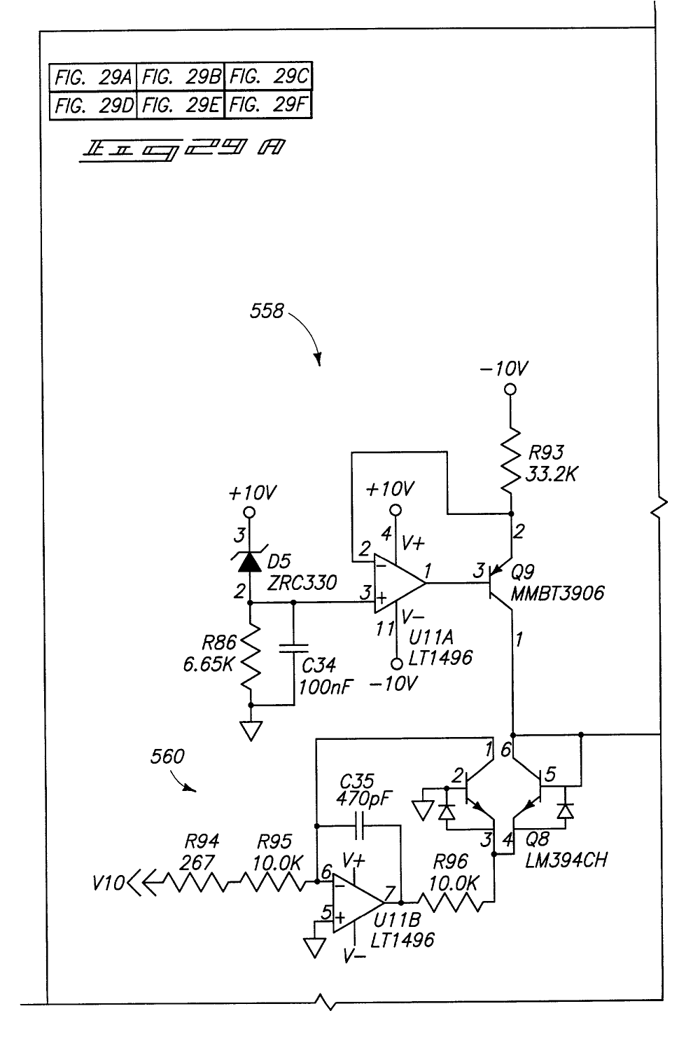

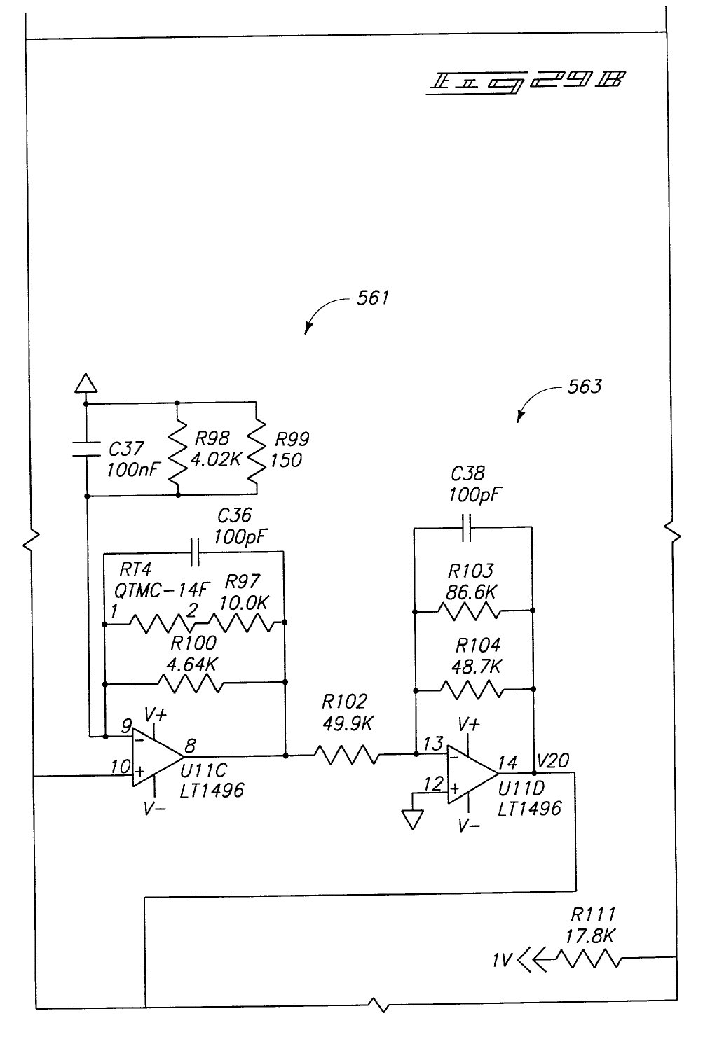

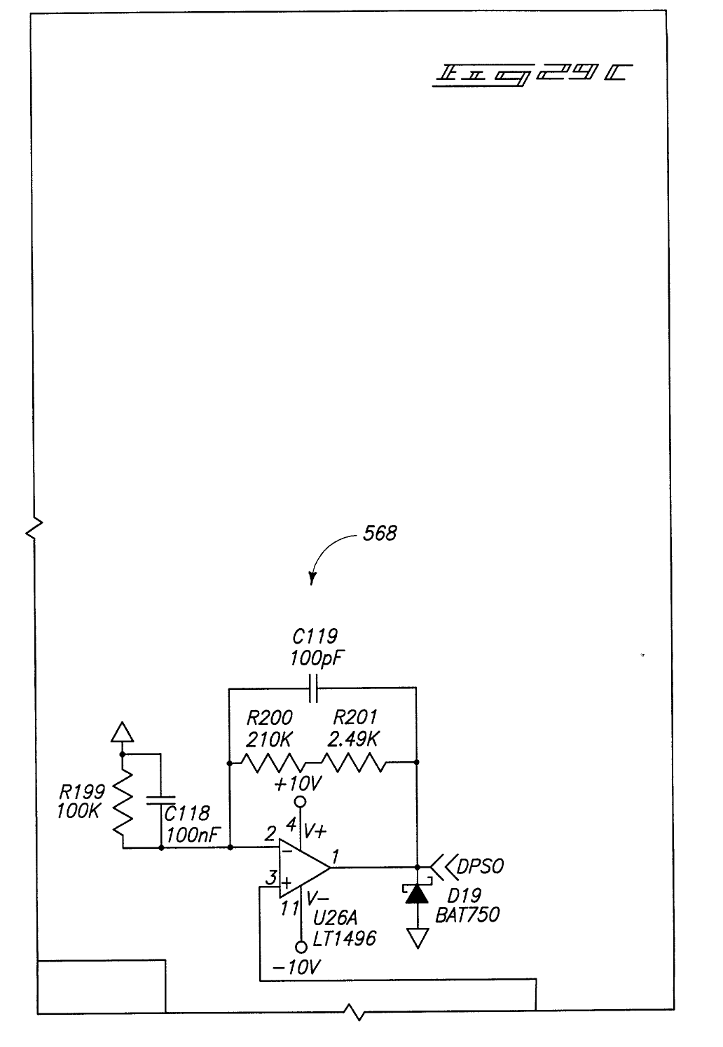

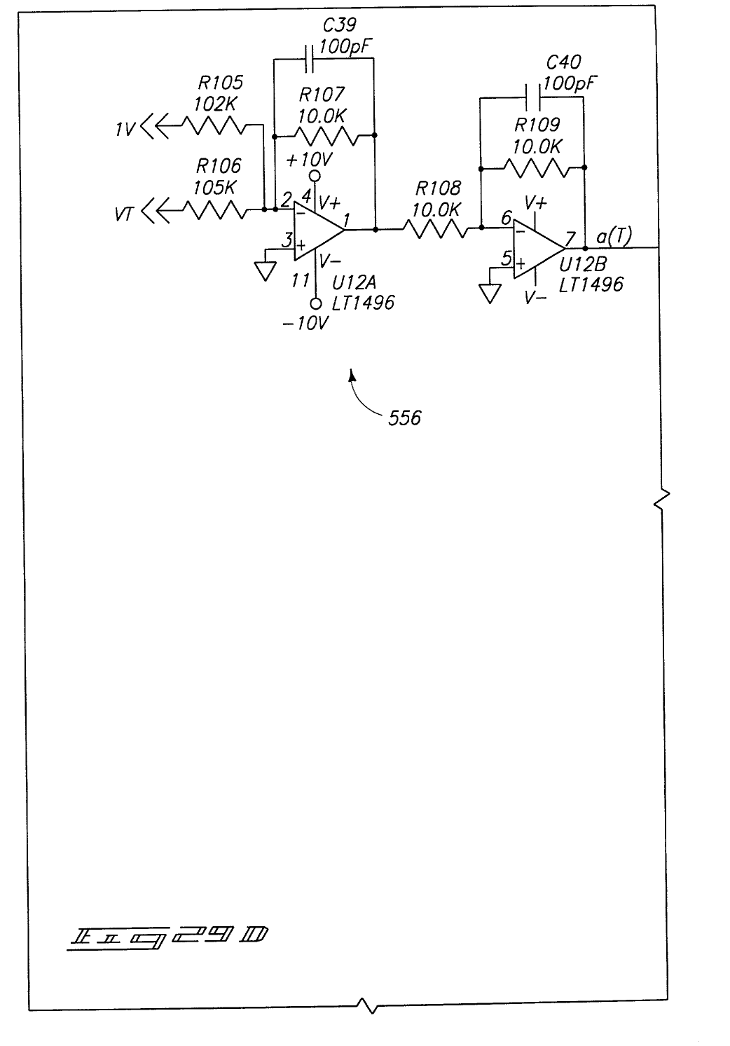

FIGS. 27A-C show construction details of one embodiment of the current supply 528. The current supply 528 includes transistor Q6 and op-amp U8A, resistors R52 and R53 coupled between a voltage source and the transistor Q6, and diode D4, resistor R51, and capacitor C22 coupled to the non-inverting input of the op-amp U8A.

FIGS. 27A-C further show anti-log amp 530 defined by op-amp U8B, associated capacitor C23, resistor R205, transistor Q5, op-amp U8C and associated resistor R59 and C25, resistors R58, R57, R68, RT2, R56, R55, R54, and capacitor C24. Resistor RT2 performs a temperature compensating function.

FIGS. 27A-C further show circuitry performing the functions of amplifiers 532 and 534 of FIGS. 21A-B. More particularly, FIGS. 27A-C includes op-amp U8D having a non-inverting input coupled to 4 volts via resistors R63 and R64 and also to a variable voltage defined by variable resistor RV3, resistor R65, resistor R66, via resistor R67, and the circuitry includes a capacitor C26. The circuitry further has, associated with op-amp U8D, resistors R60 and R61 and capacitor C27, and the inverting input of the op-amp U8D is coupled to the output of anti-log amp 530 via resistor R62 and variable resistor RV4. The circuitry shown in FIGS. 27A-C further includes a Power MOSFET Q7A, having an output defining the output voltage Vout, and having an input coupled to the output of the op-amp U8D. In the illustrated embodiment, the Power MOSFET Q7A is a model IRF7303 available from International Rectifier, 233 Kansas St., El Segundo, Calif. 90245. FIGS. 27A-C further show resistors R69, R70, R71, and C28 associated with the Power MOSFET Q7A. Power MOSFET Q7A has an input coupled to the alarm circuitry 504 via the resistor R71. The circuitry shown in FIGS. 27A-C further includes an input GATE coupled to a gate input of Power MOSFET Q7A, an input ALARM and associated resistors R69 and R70, coupled to the gate input of Power MOSFET Q7A, and an output SENSOR_OUT coupled to the source of the Power MOSFET Q7A, as well as a diode D18 coupled between SENSOR_OUT and ground.

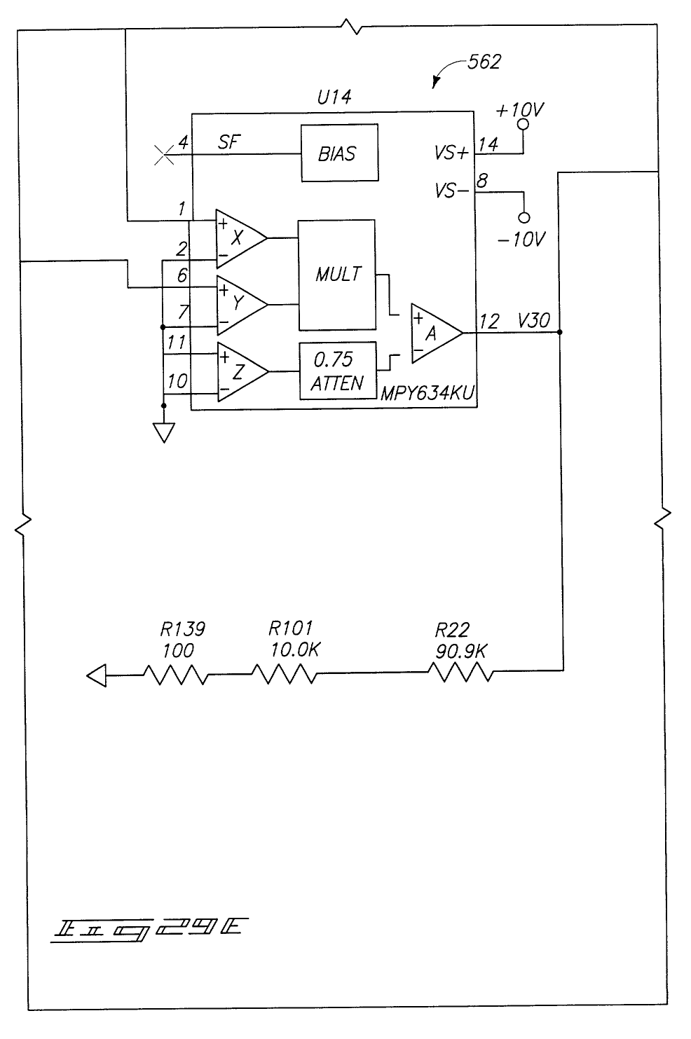

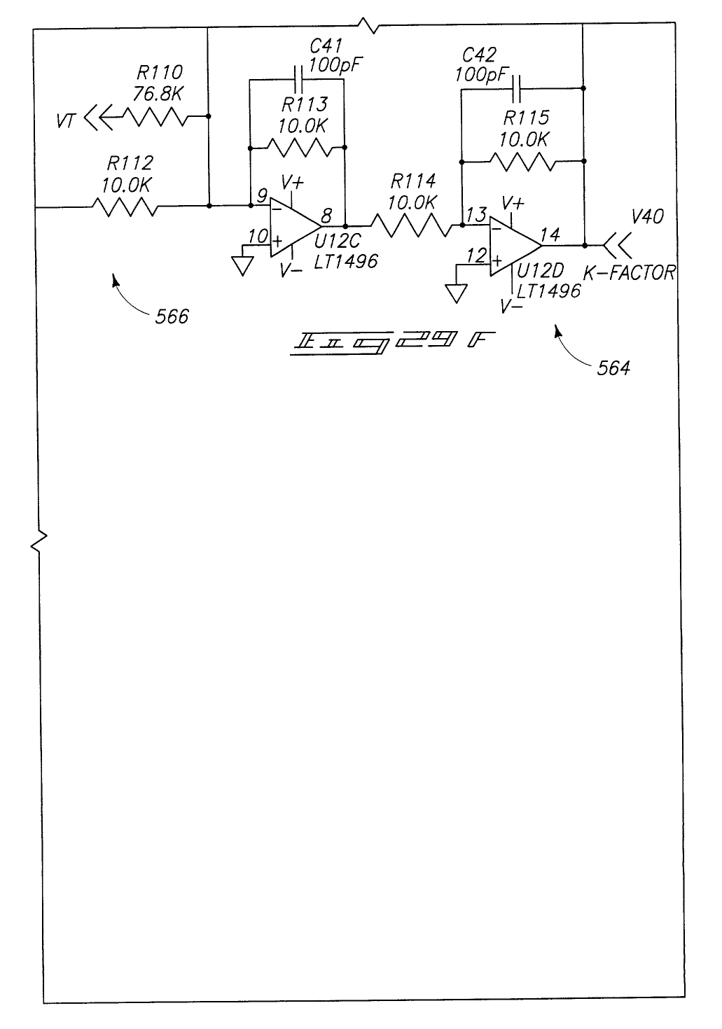

FIGS. 28A-F show construction details of circuitry for generating the voltage VH, circuitry 552 defining the function b(T), and the summing amplifier 554, in accordance with one embodiment. The circuitry for

generating the voltage VH includes a humidity sensor having an output and which, in the illustrated embodiment, is a model HIH-3610-004 available from Honeywell Sensing and Control, 11 W. Spring St., Freeport, Ill. 61032, a variable resistor RV6 coupled to the output of the humidity sensor, a temperature sensitive element RT3 coupled to the variable resistor RV6, resistors R85 and R87 associated with the temperature sensitive element RT3, inverting op-amp U10C, resistor R88 and capacitor C31 associated with the op-amp U10C, op-amp U10D and associated circuitry. In the illustrated embodiment, the temperature sensitive element RT3 is a model QTMC-14F, available from Quality Thermistor, Inc., 2108 Century Way, Boise, Id. 83709.

The op-amp U10D has associated resistors R89, R90, and associated capacitor C32, and has a non-inverting input coupled to variable resistor RV5, which is coupled to a positive voltage via resistor R91 and to a negative voltage via resistor R92. The circuitry further includes capacitor C33 coupled between the non-inverting input and ground.

FIGS. 28A-F further include construction details of one embodiment of circuitry 552 defining b(T) as 0.00845*VT-0.97545. In the illustrated embodiment, the circuitry 552 includes an op-amp U10A configured to generate an output of 1 Volt and having associated resistors R72 and R73 coupled to a voltage supply, defining a voltage divider and coupled to the non-inverting input of the op-amp U10A.

FIGS. 28A-F further include an op-amp U10B defining the summing amp 554, in one embodiment. The op-amp U10B has a non-inverting input coupled to VH via a voltage divider including resistors R83 and R84 and also to VT via resistors R80, R81 and R82. The circuitry 554 further includes a capacitor C30 in parallel with resistor R84. The non-inverting input of op-amp U10B is coupled to the 1 volt output of the op-amp U10A via a voltage divider defined by resistors R78, R79, R74, R75, and R76. The circuitry further includes a resistor R77 and parallel capacitor C29 associated with the op-amp U10B. The output of the op-amp U10B defines the voltage V10.

FIGS. 29A-F show circuitry defining the current source 558, the logarithmic amplifier 560, the multiplier 562, and circuitry defining the function a(T), in one embodiment of the invention. FIGS. 29A-F show a log amp 560 defined by an op-amp U11B having an inverting input coupled to the output of the op-amp U10B of FIGS. 28A-F, via resistors R94 and R95, having a non-inverting input coupled to ground, having an associated capacitor C35 and having an output.

The circuitry 558 shown in FIGS. 29A-F includes differential amplifier circuitry Q8 having one branch coupled between the output of the op-amp U11B via resistor R96 and the inverting input of the op-amp U11B. The circuitry 558 includes constant voltage circuitry defined by zener diode D5, resistor R86, and capacitor C34, op-amp U11A, and transistor Q9.

Transistor Q9 has a collector-emitter path coupled between a voltage source, via resistor R93, and the second branch of the differential amplifier circuitry Q8. The current source has an output defined by the collector of the second branch of the differential amplifier Q8.

FIGS. 29A-F further show construction details of one embodiment of a temperature compensating amplifier 561, associated with log amp 560, including an op-amp U11C having a non-inverting input coupled to the current source, and having temperature sensitive element RT4 associated with the op-amp U11 C, along with resistor R100, resistor R97, and capacitor C36. Capacitor C37, resistor R98, and resistor R99 are coupled together in parallel between a voltage source and the inverting input of the op-amp U11C. FIGS. 29A-F further show an inverting amp U11D, defining inverting amp 563 of FIG. 22, associated with the log amp 560, and having an inverting input coupled to the output of the op-amp U11C, via resistor R102, having a non-inverting input coupled to ground, and having associated resistors R103 and R104 and capacitor C38, and having an output defining a voltage V20.

FIGS. 29A-F further show construction details of one embodiment of circuitry 556 defining the function a(T). The circuitry 556 includes op-amp U12A having a non-inverting input coupled to ground, having an inverting input coupled to resistors R105 and R106, and having an associated resistor R107 and capacitor C39. The circuitry 556 further includes op-amp U12B having an inverting input coupled to the output of the op-amp U12A via resistor R108. Resistor 109 and capacitor C40 are coupled to each other in parallel and together are coupled between the inverting input of the op-amp U12B and the output of op-amp U12B. The non-inverting input of op-amp U12B is coupled to ground. The output of the op-amp U12B defines the function a(T).

FIGS. 29A-F further show construction details of the multiplier 562, in one embodiment. In the embodiment shown in FIGS. 29A-F, the multiplier is defined by an analog multiplier U14 which, in the illustrated embodiment, is another model MPY634KU analog multiplier available from Burr-Brown, Tucson Ariz. (now Texas Instruments). Resistors R22, R139, and R101, and capacitor C74 are associated with the analog multiplier 562. The multiplier has an input coupled to the circuitry 556 defining a(T), has an input coupled to the output of the op-amp U11D, and has an output defining a voltage V30.

FIGS. 29A-F further show circuitry including op-amps U12C and U12D, and resistors R10, R111, R112, R113, R114, and R115 and capacitors C41 and C42, defining the function c(T) and adding c(T) to the output of the multiplier 562. C(T) is defined as 0.131*VT+0.557+V30.

FIGS. 29A-F further show circuitry defining the amplifier 568 of FIG. 22. The circuitry includes op-amp U26A having a non-inverting input coupled to a voltage V40 provided by the op-amp U12D, and includes resistors R200, R201, and R199, and capacitors C118 and C119 and produces an output DPSO representative of dew point having a value, in the illustrated embodiment, of 3.125*k-factor.

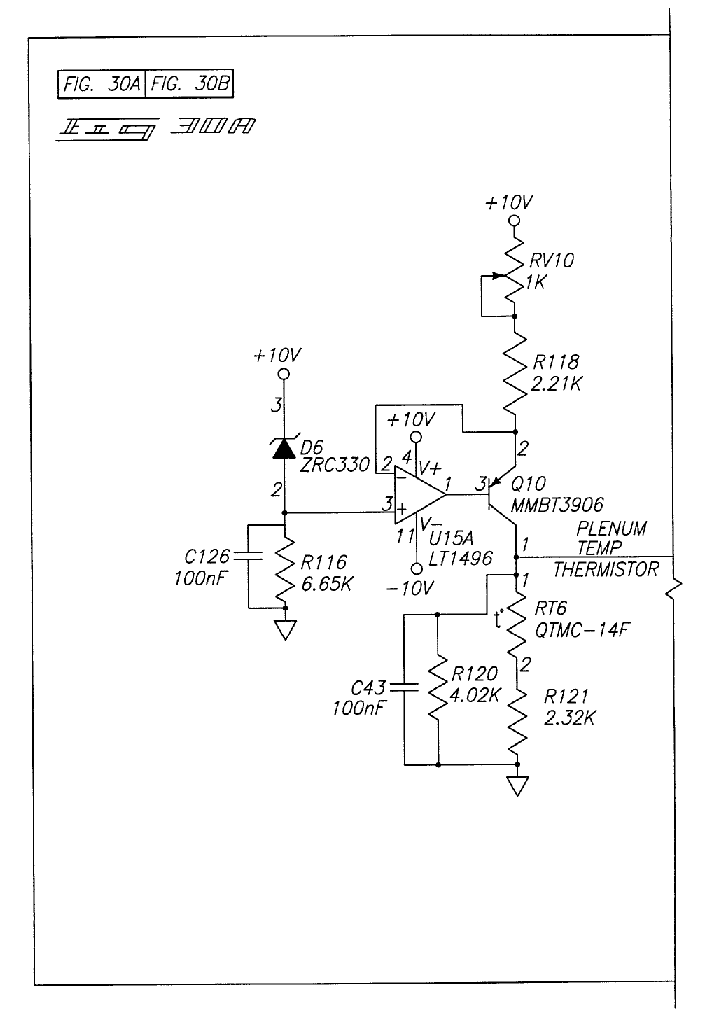

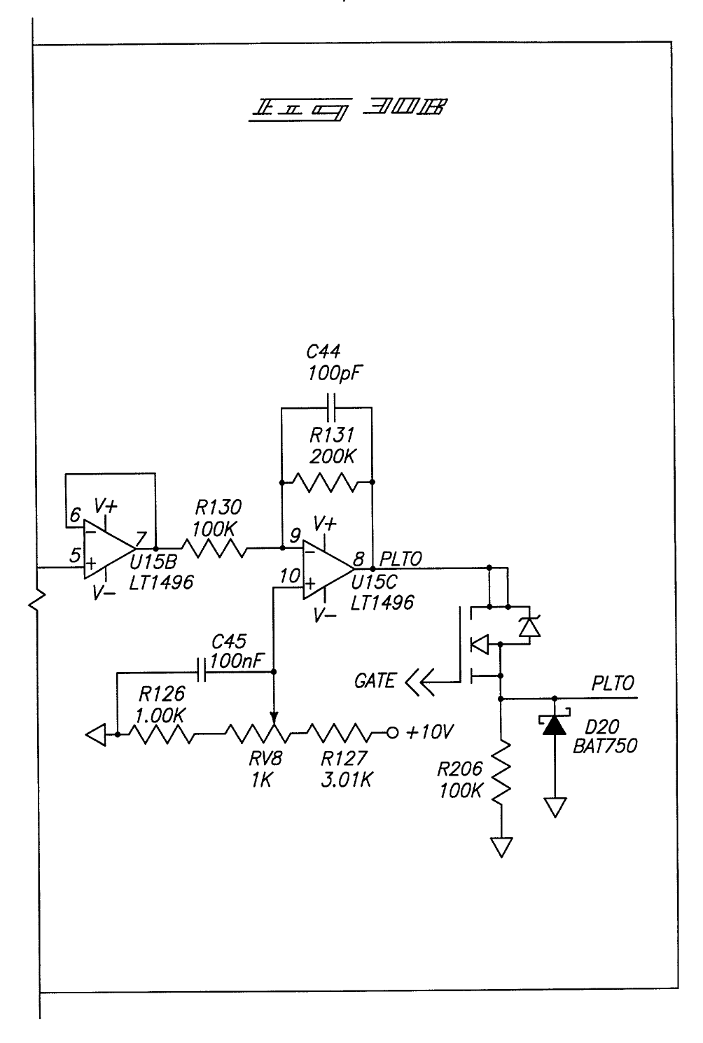

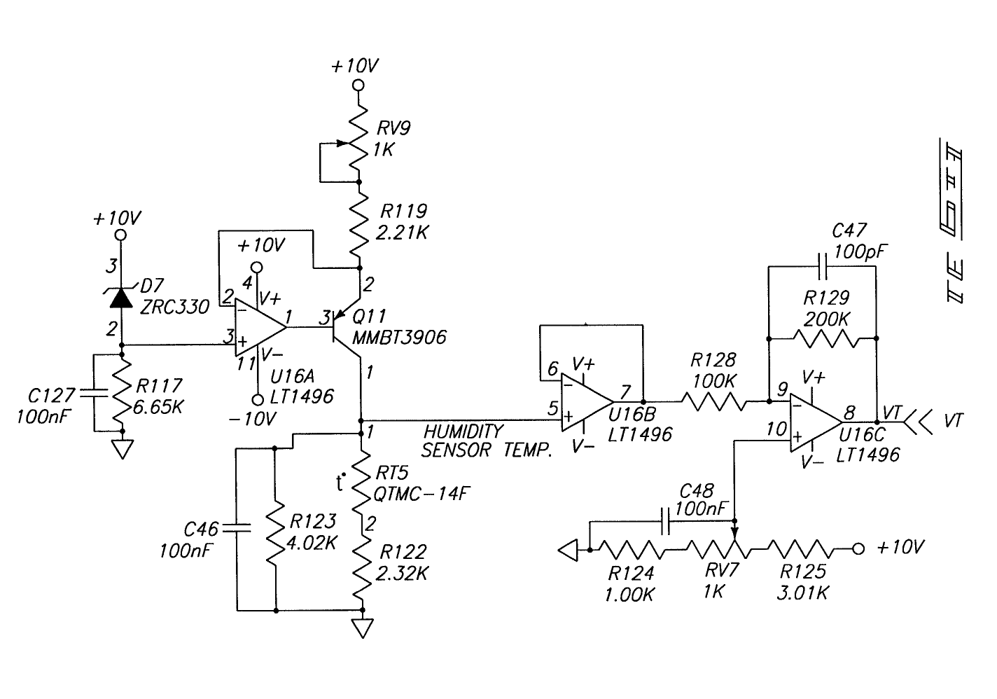

FIGS. 30A-B show construction details of circuitry for generating a signal representative of the temperature of the plenum 290, in accordance with one embodiment. The circuitry includes a zener diode D6, resistor R16 and capacitor C126 defining a voltage reference. The circuitry further includes a current source defined by transistor Q10, resistor R118, variable resistor RV10, and op-amp U15A having a non-inverting input coupled to the voltage reference, having an inverting input coupled between the transistor Q10 and the resistor R118, and having an output coupled to the gate of transistor Q10. The circuitry further includes an op-amp U15B configured as a buffer, and circuitry including temperature sensitive element RT6, resistor R121, resistor R120, and capacitor C43 coupled to the non-inverting input of the op-amp U14B and to the transistor Q10. FIGS. 30A-B further show an inverting amp with an offset, coupled to the output of the op-amp U15B, and defined by op-amp U15C, resistor R131, capacitor C44, resistor R130, variable resistor RV8, resistor R126, resistor R127, and capacitor C45. The output of the op-amp U15C provides a signal representative of the plenum temperature. FIGS. 30A-B further show a gate input and PLTO (plenum temperature) output.

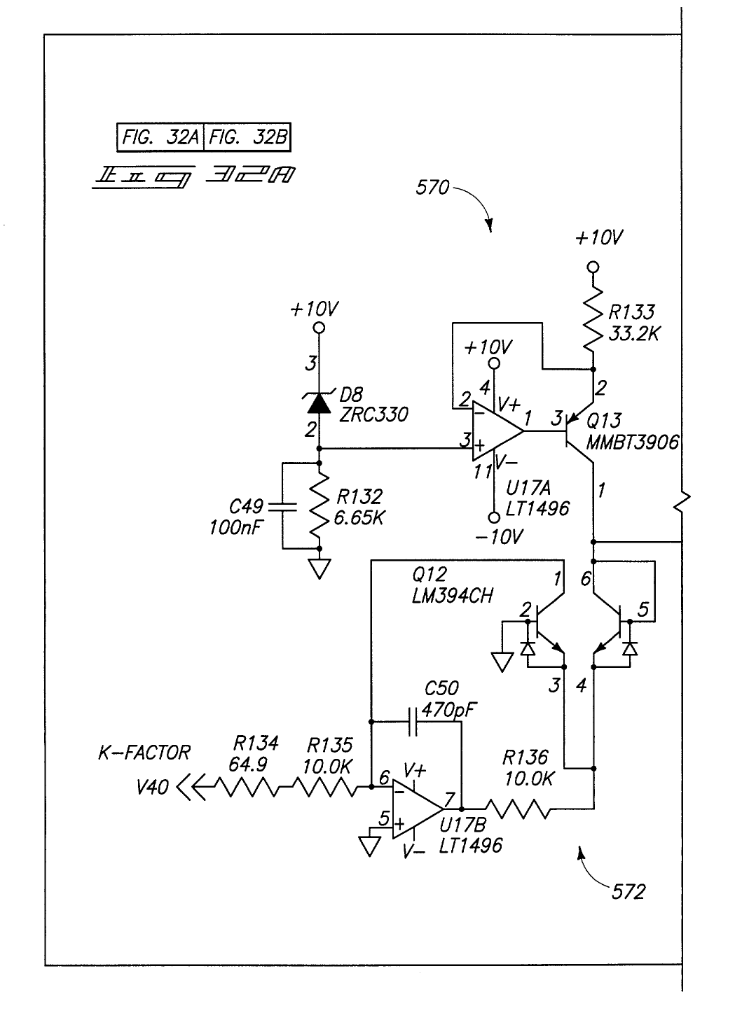

FIG. 31 shows construction details of circuitry for providing a signal representative of temperature of the humidity sensor, in accordance with one embodiment. The circuitry includes a zener diode D7, resistor R117 and capacitor C127 defining a voltage reference. The circuitry further includes a current source defined by transistor Q11, resistor R119, variable resistor RV9, and op-amp U16A having a non-inverting input coupled to the voltage reference, having an inverting input coupled between the transistor Q11 and the resistor R119, and having an output coupled to the base of transistor Q11. The circuitry of FIG. 31 further includes an op-amp U16B configured as a buffer, and circuitry including temperature sensitive element RT5, resistor R121, resistor R123, and capacitor C46 coupled to the non-inverting input of the op-amp U16B and to the transistor Q11. FIG. 31 further shows an inverting amp with an offset, coupled to the output of the op-amp U16B, and

defined by op-amp U16C, resistor R129, capacitor C47, resistor R128, variable resistor RV7, resistor R125, resistor R124, and capacitor C48. The output of the op-amp U16C provides a signal representative of the temperature of the humidity sensor.

The output voltage representing the temperature of the humidity sensor (FIG. 31) is input as VT in FIGS. 28A-F, coupled to the op-amp U10B via resistors R80, R81, and R82

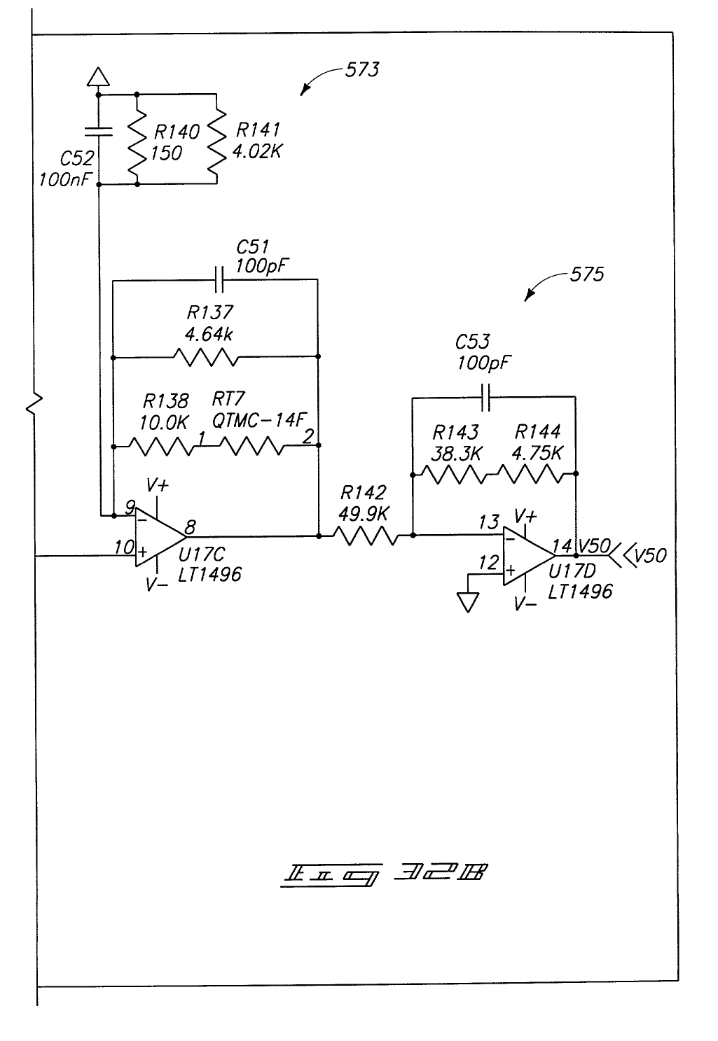

FIGS. 32A-B show construction details of circuitry defining the current source 570, the logarithmic amplifier 572, the temperature compensating amplifier 573, and the inverting amplifier 575, in one embodiment of the invention. More particularly, FIGS. 32A-B show a log amp 572 defined by an op-amp U17B having an inverting input coupled to the voltage V40 defined by the output of the op-amp U12D of FIGS. 28A-F, via resistors R134 and R135, having a non-inverting input coupled to ground, having an associated capacitor C50 and having an output.

The circuitry 572 shown in FIGS. 32A-B further includes circuitry Q12 having one branch coupled between the output of the op-amp U17B via resistor R136 and the inverting input of the op-amp U17B. The circuitry 570 includes constant voltage circuitry defined by zener diode D8, resistor R132, and capacitor C49, op-amp U17A, and transistor Q13. Transistor Q13 has a collector-emitter path coupled between a voltage source, via resistor R133, and the second branch of the differential amplifier circuitry Q13. The current source has an output defined by the collector of the second branch of the differential amplifier Q12.

FIGS. 32A-B further show construction details of one embodiment of temperature compensating amplifier 573, associated with log amp 572, including an op-amp U17C having a non-inverting input coupled to the current source, and having temperature sensitive element RT7 associated with the op-amp U17C, along with resistor R138, resistor R137, and capacitor C51. Capacitor C52, resistor R140, and resistor R141 are coupled together in parallel between a voltage source and the inverting input of the op-amp U17C. FIGS. 32A-B further show an inverting amp U17D, defining inverting amp 575 of FIGS. 32A-B, associated with the log amp 572, and having an inverting input coupled to the output of the op-amp U17C, via resistor R142, having a non-inverting input coupled to ground, and having associated resistors R143 and R144 and capacitor C53, and having an output defining a voltage V50.

Various voltage sources shown in the schematics, e.g., V5V and V5P are 5 volt voltage sources, in the illustrated embodiment.

Thus, a method and apparatus have been provided for calculating dew point from temperature and humidity using analog or digital circuitry, and a method and apparatus have been provided for compensating a MOS gas sensor for the effects of temperature and humidity in a simplified fashion.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. A method of calculating dew point, comprising:
providing a signal representative of relative humidity of the ambient;
calculating a temperature signal representative of a predetermined dew point;
providing a signal representative of actual temperature of the ambient;
determining the difference between the signal representative of actual temperature and the calculated temperature signal to provide a differential temperature;
using the relative humidity signal, calculating the rate at which dew point changes as a function of temperature; and
calculating dew point by multiplying the differential temperature by the calculated rate at which dew point changes as a function of temperature.

2. A method of calculating dew point in accordance with claim 1 wherein providing a signal representative of relative humidity of the ambient comprises measuring the relative humidity of the ambient using a humidity sensor.

3. A method of calculating dew point in accordance with claim 1 wherein providing a signal representative of actual temperature of the ambient comprises measuring the temperature of the ambient using a temperature sensor.

4. A method of calculating dew point in accordance with claim 1 wherein determining the difference between the signal representative of actual temperature and the calculated temperature signal is performed using a differential amplifier.

5. A method of calculating dew point in accordance with claim 1 wherein calculating the rate at which dew point changes as a function of temperature is performed using an analog log amplifier coupled to the humidity signal; a multiplier configured to take the log of the square of the humidity signal; and amplifiers respectively coupled to the multipier and to the humidity signal.

6. A method of calculating dew point in accordance with claim 1 wherein calculating the rate at which dew point changes as a function of temperature is performed using the formula τ=aτlog(H)ˆ 2+bτlog(H)+cτ wherein h is relative humidity and wherein aτ, bτ, and cτ are constants, and τ is temperature at which relative humidity correlates to a predetermined dew point.

7. A method of calculating dew point in accordance with claim 1 wherein calculating the rate at which dew point changes as a function of temperature is performed using the formula τ=aτlog(H)ˆ 2+bτlog(H)+cτ wherein h is relative humidity and wherein aτ is 5.65, bτ is −44.3, cτ is 66.14, and τ is temperature, expressed in Kelvins beyond a −20° C. baseline, at which H correlates to a dew point of −20° C.

8. A method of calculating dew point in accordance with claim 1 wherein calculating dew point by multiplying the differential temperature by the calculated rate at which dew point changes as a function of temperature is performed using a multiplier.

9. A method of calculating dew point in accordance with claim 1 wherein the steps of calculating a temperature signal representative of a predetermined dew point; determining the difference; and calculating the rate at which dew point changes as a function of temperature, are all performed by analog circuitry.

10. A method of calculating dew point in accordance with claim 1 wherein the steps of calculating a temperature signal representative of a predetermined dew point; determining the difference; and calculating the rate at which dew point changes as a function of temperature is performed essentially by analog circuitry.

11. A method of calculating dew point in accordance with claim 1 wherein the steps of calculating a temperature signal representative of a predetermined dew point; determining the difference; and calculating the rate at which dew point changes as a function of temperature are performed digitally.

12. A method of calculating dew point in accordance with claim 1 wherein the steps of calculating a temperature signal representative of a predetermined dew point; determining the difference; and calculating the rate at which dew point changes as a function of temperature are performed essentially using digital equipment.

13. A method of calculating dew point in accordance with claim 1 wherein determining the difference between the signal representative of actual temperature and the calculated temperature signal comprises subtracting the signal representative of actual temperature from the calculated temperature signal.

14. A method of calculating dew point in accordance with claim 1 wherein determining the difference between the signal representative of actual temperature and the calculated temperature signal comprises subtracting the calculated temperature signal from the signal representative of actual temperature.

15. A method of calculating and compensating for dew point, comprising:
providing a signal representative of relative humidity of the ambient;
calculating a temperature signal representative of a predetermined dew point;
providing a signal representative of actual temperature of the ambient and determining the difference between the signal representative of actual temperature and the calculated temperature signal to provide a differential temperature;
using the relative humidity signal, calculating the rate at which dew point changes as a function of temperature;
calculating dew point by multiplying the differential temperature by the calculated rate at which dew point changes as a function of temperature;
providing a signal indicative of gas concentration of a target gas in an ambient; and
modifying the signal indicative of gas concentration using the calculated dew point to simultaneously compensate for the effects of both temperature and relative humidity on a MOS gas sensor.

16. A method of calculating and compensating for dew point in accordance with claim 15 wherein providing a signal representative of gas concentration comprises measuring the gas concentration using a MOS sensor.

17. A method of calculating and compensating for dew point in accordance with claim 15 wherein providing a signal representative of relative humidity of the ambient comprises measuring the relative humidity of the ambient using a humidity sensor.

18. A method of calculating and compensating for dew point in accordance with claim 15 wherein providing a signal representative of actual temperature of the ambient comprises measuring the temperature of the ambient using a temperature sensor.

19. A method of calculating and compensating for dew point in accordance with claim 15 wherein determining the difference between the signal representative of actual temperature and the calculated temperature signal is performed using a differential amplifier.

20. A method of calculating and compensating for dew point in accordance with claim 15 wherein calculating the rate at which dew point changes as a function of temperature is performed using an analog log amplifier coupled to the humidity signal; a multiplier configured to take the log of the square of the humidity signal; and amplifiers respectively coupled to the multipier and to the humidity signal.

21. A method of calculating and compensating for dew point in accordance with claim 15 wherein calculating the rate at which dew point changes as a function of temperature is performed using the formula τ=aτlog(H)ˆ 2+bτlog(H)+cτ, and wherein H is relative humidity, and wherein aτ, bτ, and cτ are constants, and τ is temperature at which relative humidity correlates to a predetermined dew point.

22. A method of calculating and compensating for dew point in accordance with claim 15 wherein calculating the rate at which dew point changes as a function of temperature is performed using the formula τ=aτlog(H)ˆ 2+bτlog(H)+cτ, and wherein H is relative humidity, and wherein aτ is 5.65, bτ is −44.3, cτ is 66.14, and τ is temperature, in Kelvins beyond a −20° C. baseline, at which H correlates to a dew point of −20° C.

23. A method of calculating and compensating for dew point in accordance with claim 15 wherein calculating dew point by multiplying the differential temperature by the calculated rate at which dew point changes as a function of temperature is performed using a multiplier.

24. A method of calculating and compensating for dew point in accordance with claim 15 wherein calculating a temperature signal representative of a predetermined dew point; determining the difference; and calculating the rate at which dew point changes as a function of temperature are all performed by analog circuitry.

25. A method of calculating and compensating for dew point in accordance with claim 15 wherein the steps of calculating a temperature signal representative of a predetermined dew point; determining the difference; and calculating the rate at which dew point changes as a function of temperature is performed essentially by analog circuitry.

26. A method of calculating and compensating for dew point in accordance with claim 15 wherein calculating a temperature signal representative of a predetermined dew point; determining the difference; and calculating the rate at which dew point changes as a function of temperature are performed digitally.

27. A method of calculating and compensating for dew point in accordance with claim 15 wherein said calculating a temperature signal representative of a predetermined dew point; said determining the difference; and said calculating the rate at which dew point changes as a function of temperature calculating dew point are performed essentially using digital equipment.

28. A system for calculating dew point, comprising:
a humidity sensor which, in operation, provides a signal representative of relative humidity of the ambient;
circuitry configured to calculate a temperature signal representative of a predetermined dew point;
a temperature sensor which, in operation, provides a signal representative of temperature of the ambient;
circuitry which, in operation, determines the difference between the calculated temperature signal and the signal representative of the temperature of the ambient to provide a differential temperature;
circuitry which, in operation, using the relative humidity signal, calculates the rate at which dew point changes as a function of temperature; and
circuitry which, in operation, calculates dew point by multiplying the differential temperature by the calculated rate at which dew point changes as a function of temperature.

29. A system for calculating dew point in accordance with claim 28 wherein the circuitry which, in operation, subtracts the calculated temperature signal from the signal representative of actual temperature comprises a differential amplifier.

30. A system for calculating dew point in accordance with claim 28 wherein the circuitry which, in operation, calculates the rate at which dew point changes as a function of temperature comprises an analog log amplifier coupled to the humidity sensor; a multiplier configured to take the log of the square of the humidity signal; and amplifiers respectively coupled to the multipier and to the humidity signal.

31. A system for calculating dew point in accordance with claim 28 wherein the circuitry which, in operation, calculates the rate at which dew point changes as a function of temperature uses the formula τ=aτlog(H)ˆ 2+bτlog(H)+cτ and wherein H is relative humidity, and wherein aτ, bτ, and cτ are constants, and T is temperature at which relative humidity correlates to a predetermined dew point.

32. A system for calculating dew point in accordance with claim 28 wherein the circuitry which, in operation, calculates the rate at which dew point changes as a function of temperature uses the formula τ=aτlog(H)ˆ 2+bτlog(H)+cτ, wherein h is relative humidity and wherein aτ is 5.65, bτ is −44.3, cτ is 66.14, and τ is temperature, in Kelvins beyond a −20° C. baseline, at which H correlates to a dew point of −20° C.

33. A system for calculating dew point in accordance with claim 28 wherein the circuitry which, in operation, calculates dew point by multiplying the differential temperature by the calculated rate at which dew point changes as a function of temperature comprises an analog multiplier.

34. A system for calculating dew point in accordance with claim 28 wherein the circuitry which, in operation, calculates dew point by multiplying the differential temperature by the calculated rate at which dew point changes as a function of temperature comprises digital circuitry.

35. A system for calculating dew point in accordance with claim 28 wherein the circuitry which, in operation, calculates a temperature signal representative of a predetermined dew point; determines the difference between the calculated temperature signal and the signal representative of the temperature of the ambient; and calculates the rate at which dew point changes as a function of temperature calculating dew point all consist essentially of analog circuitry.

36. A system for calculating dew point in accordance with claim 28 wherein said circuitry which, in operation, calculates a temperature signal representative of a predetermined dew point; determines the difference between the calculated temperature signal and the signal representative of the temperature of the ambient; and calculates the rate at which dew point changes as a function of temperature calculating dew point respectively comprise analog circuitry.

37. A system for calculating dew point in accordance with claim 28 wherein said circuitry which, in operation, calculates a temperature signal representative of a predetermined dew point; determines the difference between the calculated temperature signal and the signal representative of the temperature of the ambient; and calculates the rate at which dew point changes as a function of temperature calculating dew point respectively comprise digital circuitry.

38. A system for calculating dew point in accordance with claim 28 wherein said circuitry which, in operation, calculates a temperature signal representative of a predetermined dew point; determines the difference between the calculated temperature signal and the signal representative of the temperature of the ambient; and calculates the rate at which dew point changes as a function of temperature calculating dew point are all defined by a processor.

39. A MOS gas sensor system comprising:
a MOS gas sensor which, in operation, provides a signal representative of the concentration of a target gas in an ambient;
a humidity sensor which, in operation, provides a signal representative of relative humidity of the ambient;
circuitry configured to calculate a temperature signal representative of a predetermined dew point;
a temperature sensor which, in operation, provides a signal representative of temperature of the ambient;
circuitry which, in operation, determines the difference between the calculated temperature signal and the signal representative of the temperature of the ambient to provide a differential temperature;
circuitry which, in operation, using the relative humidity signal, calculates the rate at which dew point changes as a function of temperature;
dew point calculation circuitry which, in operation, calculates dew point by multiplying the differential temperature by the calculated rate at which dew point changes as a function of temperature; and
compensation circuitry coupled to the dew point calculation circuitry and configured to modify the signal from the MOS gas sensor, using the calculated dew point, to simultaneously compensate for the effects of both temperature and relative humidity on the MOS gas sensor.

40. A MOS gas sensor system in accordance with claim 39 wherein the circuitry configured to modify the signal from the gas sensor comprises analog circuitry.

41. A MOS gas sensor system in accordance with claim 39 wherein the circuitry configured to modify the signal from the gas sensor consists essentially of analog circuitry.

42. A fuel cell system comprising:
a housing having a fuel gas inlet and an exhaust outlet;
at least one ion exchange fuel cell membrane located within the housing; and
a MOS gas sensor system including:
a MOS gas sensor which, in operation, provides a signal representative of the concentration of a target gas in an ambient;
a humidity sensor which, in operation, provides a signal representative of relative humidity of the ambient circuitry configured to calculate a temperature signal representative of a predetermined dew point;
a temperature sensor which, in operation, provides a signal representative of the temperature of the ambient;
circuitry which, in operation, determines the difference between the calculated temperature signal and the signal representative of the temperature of the ambient to provide a differential temperature;
circuitry which, in operation, using the relative humidity signal, calculates the rate at which dew point changes as a function of temperature;
dew point calculation circuitry which, in operation, calculates dew point by multiplying the differential temperature by the calculated rate at which dew point changes as a function of temperature; and
compensation circuitry coupled to the dew point calculation circuitry and configured to modify the signal from the MOS gas sensor, using the calculated dew point, to simultaneously compensate for the effects of both temperature and relative humidity on the MOS gas sensor.

43. A fuel cell system in accordance with claim 42 and further comprising a fuel supply coupled to the fuel supply inlet of the housing.

44. A fuel cell system in accordance with claim 43 wherein the fuel supply comprises hydrogen gas, and wherein the MOS sensor of the MOS gas sensor system is configured to sense the concentration of hydrogen gas.

44. A fuel cell system in accordance with claim 43 and further comprising a controller electrically coupled with the ion exchange membrane, the controller defining the compensation circuitry.

45. A fuel cell system in accordance with claim 43 and further comprising a controller electrically coupled with the ion exchange membrane, the controller defining the circuitry configured to calculate a temperature signal representative of a predetermined dew point; the circuitry which, in operation, determines the difference between the calculated temperature signal and the signal representative of the temperature of the ambient to provide a differential temperature; the circuitry which, in operation, calculates the rate at which dew point changes as a function of temperature; the dew point calculation circuitry; and the compensation circuitry.

46. A method of calculating dew point, comprising:
providing a signal representative of relative humidity of the ambient;
providing a signal representative of temperature of the ambient; and
calculating the rate at which dew point changes as a function of temperature and humidity using analog circuitry modeled on a least squares fit to the formula τ=aτlog(H)ˆ 2+bτlog(H)+cτ wherein H is relative humidity and wherein aτ, bτ, and cτ are constants, and τ is temperature at which relative humidity correlates to a predetermined dew point.

47. A method of calculating dew point in accordance with claim 46 wherein providing a signal representative of relative humidity of the ambient comprises measuring the relative humidity of the ambient using a humidity sensor.

48. A method of calculating dew point in accordance with claim 46 wherein providing a signal representative of temperature of the ambient comprises measuring the temperature of the ambient using a temperature sensor.

49. Analog circuitry for calculating dew point, comprising:
a humidity sensor configured to provide a signal representative of relative humidity of the ambient;
a temperature sensor configured to provide a signal representative of temperature of the ambient; and
analog circuitry, coupled to the humidity sensor and the temperature sensor, configured to provide a signal representative of dew point using analog circuitry modeled on a least squares fit to a formula that relates temperature and relative humidity to dew point.

50. Analog circuitry for calculating dew point, in accordance with claim 49, wherein the analog circuitry implements a least squares fit to a surface.

51. Analog circuitry for calculating dew point, in accordance with claim 49, wherein the analog circuitry is modeled on a least squares fit to a formula that includes a logarithmic function.

52. A method of calculating dew point, comprising:
providing a signal representative of relative humidity of the ambient;
providing a signal representative of temperature of the ambient; and
calculating the rate at which dew point changes as a function of temperature and humidity using analog circuitry modeled to implement an equation of the form
K(T,H)=(a₁T+a₀)log₁₀[(b₁T+b₀)+H]+(c₁T+c₀)
where K(T,H) is a scaled dew point temperature, where T is ambient temperature, where a₀, a₁, b₀, b₁, c₀, and c₁ are constants, and where H is relative humidity.

53. A method of calculating dew point in accordance with claim 52 wherein providing a signal representative of relative humidity of the ambient comprises measuring the relative humidity of the ambient using a humidity sensor.

54. A method of calculating dew point in accordance with claim 52 wherein providing a signal representative of temperature of the ambient comprises measuring the temperature of the ambient using a temperature sensor.

55. A method of calculating dew point, comprising:
sensing the relative humidity of the ambient;
sensing the temperature of the ambient; and
determining the rate at which dew point changes as a function of temperature and humidity using a transfer function of the form Y=MX+B which can be plotted as a line segment on a graph having x and y axes,
wherein b is a first-order function of temperature that determines the y-axis origin of the line segment mx, and represents a reference point on a dew point scale;
wherein x is a multi-variable representation of x-axis displacement from the origin and is a first-order log (base 10) function of both temperature and humidity;
wherein m is a function that determines the slope of the line segment and is a first-order function of temperature; and
wherein y is the difference relative to b and represents dew point temperature.

56. A method of calculating dew point in accordance with claim 55 wherein each of the terms m and b is of the form L₁T+L₀ where both L₁ and L₀ are constants and T is temperature.

57. A method of calculating dew point in accordance with claim 55 wherein x is a function of the form log₁₀[(b₁T+b₀)+H] where b₁ is a constant, b₀ is a constant, T is the sensed temperature, and H is the sensed relative humidity.

58. A method of calculating dew point in accordance with claim 55 wherein m is of the form a₁T+a₀, wherein x is of the form log₁₀[(b₁T+b₀)+h] and wherein b is of the form c₁T+c₀.

59. A method of calculating dew point in accordance with claim 55 wherein determining the rate at which dew point changes as a function of temperature and humidity comprises solving the formula: t′=(a₁T+a₀)log₁₀[(b₁T+b₀)+H]+(c₁T+c₀) where t′ is dew point, T is temperature, H is relative humidity, and a₀, a₁, b₀, b₁, c₁, and co are constants, using analog circuitry.

60. A method of calculating dew point in accordance with claim 55 wherein determining the rate at which dew point changes as a function of temperature and humidity comprises digitally solving the formula: t′=(a₁T+a₀)log₁₀[(b₁T+b₀)+H]+(c₁T+co) where t′ is dew point, T is temperature, H is relative humidity, and a₀, a₁, b₀, b₁, c₁, and c₀ are constants.

61. A method of compensating a MOS gas sensor for environmental dependencies, where said MOS gas sensor has a transfer function that is substantially linear when viewed on a log₁₀−log₁₀ plot of a signal representative of the sensor's output versus gas concentration, the transfer function having a slope representing raw gain and having a raw offset, the method comprising:
taking the raw offset and gain of the sensor's transfer function, and generating a first signal by normalizing the raw offset and gain to fit a predetermined transfer function at a predetermined dew point, and wherein the sensor's first signal's gain is scaled on a log₁₀ basis;
taking a second signal, which is a function of the dew point of the gas which the MOS gas sensor is configured to sense, and where the influence of said second signal on the first signal is null at a predetermined dew point; and
summing the first and second signals so as to offset the first signal upwards or downwards as viewed on a log₁₀−log₁₀ plot.

62. A method in accordance with claim 61 wherein the first and second signals are generated using analog circuitry.

63. A method in accordance with claim 62 wherein the summing is performed using analog circuitry.

64. A method in accordance with claim 61 wherein the first and second signals are generated using digital equipment.

65. A method in accordance with claim 64 wherein the summing is performed using digital equipment.

66. A method of compensating a MOS gas sensor for environmental dependencies, where said MOS gas sensor has a transfer function that is substantially linear when viewed on a log₁₀−log₁₀ plot of a signal representative of the sensor's output versus gas concentration, the transfer function having a slope representing raw gain and having a raw offset, the method comprising:
taking the raw offset and gain of the sensor's transfer function, and generating a first signal by normalizing the raw offset and gain to fit a predetermined transfer function at a predetermined dew point, and wherein the sensor's first signal's gain is scaled on a log₁₀ basis;
taking a second signal, which is a function of the dew point of the gas which the MOS gas sensor is configured to sense, and where the influence of said second signal on the first signal is null at a predetermined dew point; and
summing the first and second signals so as to offset the first signal upwards or downwards as viewed on a log₁₀−log₁₀ plot.

67. A method in accordance with claim 66 wherein the first and second signals are generated using analog circuitry.

68. A method in accordance with claim 67 wherein the summing is performed using analog circuitry.

69. A method in accordance with claim 66 wherein the first and second signals are generated digitally.

70. A method in accordance with claim 69 wherein the summing is performed digitally.

71. A method in accordance with claim 66 wherein the normalizing comprises varying the gain of an amplifier such that the output of the amplifier, when the sensor is exposed to a predetermined concentration of gas, is at a predetermined level.

72. A method in accordance with claim 71 wherein the normalizing comprises varying the gain of an amplifier such that the output of the amplifier, when the sensor is exposed to a second predetermined concentration of gas, different from the first predetermined concentration of gas, is at a second predetermined voltage, different from the first predetermined voltage.

73. A method of calculating dew point (t′) given inputs of relative humidity (H) and temperature (T), using a transfer function t′=MX+B; wherein:
B is a function of temperature;
M is a function of temperature; and
X is a log base 10 function of relative humidity and temperature.

74. A method in accordance with claim 73, wherein
B=c₁T+c₀;
M=a₁T+a₀;
X=log₁₀[(b₁T+b₀)+V_{H}]; and
a₀, a₁, b₀, b₁, c₀, and c₁ are constants.

75. A method in accordance with claim 74 wherein the following formula is solved to calculate dew point: t′=(a₁T+a₀)log₁₀[(b₁T+b₀)+V_{H}]+c₁T+c₀.

76. A method in accordance with claim 73 and implemented using analog circuitry.

77. A method in accordance with claim 73 and implemented digitally.
